SEQUENCE LISTING (261 sequences)

SEQ ID NO: 1 - protein, 5 aa, Artificial Sequence
  Synthetic polypeptide
YYWMN

SEQ ID NO: 2 - protein, 5 aa, Artificial Sequence
  Synthetic polypeptide
NYGMN

SEQ ID NO: 3 - protein, 5 aa, Artificial Sequence
  Synthetic polypeptide
NYGMN

SEQ ID NO: 4 - protein, 5 aa, Artificial Sequence
  Synthetic polypeptide
SYGMH

SEQ ID NO: 5 - protein, 8 aa, Artificial Sequence
  Synthetic polypeptide
GFTFSYYW

SEQ ID NO: 6 - protein, 8 aa, Artificial Sequence
  Synthetic polypeptide
GYTFTNYG

SEQ ID NO: 7 - protein, 8 aa, Artificial Sequence
  Synthetic polypeptide
GFTFSYYW

SEQ ID NO: 8 - protein, 8 aa, Artificial Sequence
  Synthetic polypeptide
GFTFSSYG

SEQ ID NO: 9 - protein, 5 aa, Artificial Sequence
  Synthetic polypeptide
SSTMH

SEQ ID NO: 10 - protein, 5 aa, Artificial Sequence
  Synthetic polypeptide
NYWIH

SEQ ID NO: 11 - protein, 10 aa, Artificial Sequence
  Synthetic polypeptide
GYTFTDYYIY

SEQ ID NO: 12 - protein, 5 aa, Artificial Sequence
  Synthetic polypeptide
DYYMA

SEQ ID NO: 13 - protein, 5 aa, Artificial Sequence
  Synthetic polypeptide
SYGIH

SEQ ID NO: 14 - protein, 5 aa, Artificial Sequence
  Synthetic polypeptide
SYGIS

SEQ ID NO: 15 - protein, 5 aa, Artificial Sequence
  Synthetic polypeptide
SYGLS

SEQ ID NO: 16 - protein, 5 aa, Artificial Sequence
  Synthetic polypeptide
SYGLS

SEQ ID NO: 17 - protein, 5 aa, Artificial Sequence
  Synthetic polypeptide
SYGLS

SEQ ID NO: 18 - protein, 5 aa, Artificial Sequence
  Synthetic polypeptide
SYGIS

SEQ ID NO: 19 - protein, 5 aa, Artificial Sequence
  Synthetic polypeptide
NFVMS

SEQ ID NO: 20 - protein, 5 aa, Artificial Sequence
  Synthetic polypeptide
TYGMH

SEQ ID NO: 21 - protein, 5 aa, Artificial Sequence
  Synthetic polypeptide
SYGMH

SEQ ID NO: 22 - protein, 5 aa, Artificial Sequence
  Synthetic polypeptide
SYAMS

SEQ ID NO: 23 - protein, 19 aa, Artificial Sequence
  Synthetic polypeptide
EIRLKSNNYATHYAESVKG

SEQ ID NO: 24 - protein, 17 aa, Artificial Sequence
  Synthetic polypeptide
WLNTYTGESIYPDDFKG

SEQ ID NO: 25 - protein, 17 aa, Artificial Sequence
  Synthetic polypeptide
WLNTYTGESWYPDDFKG

SEQ ID NO: 26 - protein, 17 aa, Artificial Sequence
  Synthetic polypeptide
VIWYDGSNYYYTDSVKG

SEQ ID NO: 27 - protein, 10 aa, Artificial Sequence
  Synthetic polypeptide
IRLKSNNYAT

SEQ ID NO: 28 - protein, 8 aa, Artificial Sequence
  Synthetic polypeptide
LNTYTGES

SEQ ID NO: 29 - protein, 10 aa, Artificial Sequence
  Synthetic polypeptide
IRLKSNNYAT

SEQ ID NO: 30 - protein, 8 aa, Artificial Sequence
  Synthetic polypeptide
IWYDGSNY

SEQ ID NO: 31 - protein, 16 aa, Artificial Sequence
  Synthetic polypeptide
LIGSGGGIYYGDSVKG

SEQ ID NO: 32 - protein, 17 aa, Artificial Sequence
  Synthetic polypeptide
VIDPSDTYPNYNKKFKG

SEQ ID NO: 33 - protein, 19 aa, Artificial Sequence
  Synthetic polypeptide
WIFPGTGNTYYNENFKDKA

SEQ ID NO: 34 - protein, 17 aa, Artificial Sequence
  Synthetic polypeptide
SISYDGSNKYYGDSVKG

SEQ ID NO: 35 - protein, 17 aa, Artificial Sequence
  Synthetic polypeptide
VIGYDGSDKNYADSVKG

SEQ ID NO: 36 - protein, 17 aa, Artificial Sequence
  Synthetic polypeptide
WISAYNGNTKYAQKLQG

SEQ ID NO: 37 - protein, 17 aa, Artificial Sequence
  Synthetic polypeptide
WISPYNGNTHYAQKLQG

SEQ ID NO: 38 - protein, 17 aa, Artificial Sequence
  Synthetic polypeptide
WISPYNGNTHYAQKLQG

SEQ ID NO: 39 - protein, 17 aa, Artificial Sequence
  Synthetic polypeptide
WISAYNGNTNYAQKLQG

SEQ ID NO: 40 - protein, 17 aa, Artificial Sequence
  Synthetic polypeptide
WISAYNGNTKYAQKLQG

SEQ ID NO: 41 - protein, 17 aa, Artificial Sequence
  Synthetic polypeptide
GISGSGGNTDHADSVKG

SEQ ID NO: 42 - protein, 17 aa, Artificial Sequence
  Synthetic polypeptide
VISHDGSDKYYADSVKG

SEQ ID NO: 43 - protein, 17 aa, Artificial Sequence
  Synthetic polypeptide
VIWYDGSIKYYADSVKG

SEQ ID NO: 44 - protein, 17 aa, Artificial Sequence
  Synthetic polypeptide
AISDSGGSTYYADSVKG

SEQ ID NO: 45 - protein, 9 aa, Artificial Sequence
  Synthetic polypeptide
RDEYYAMDY

SEQ ID NO: 46 - protein, 11 aa, Artificial Sequence
  Synthetic polypeptide
GDYGYDDPLDY

SEQ ID NO: 47 - protein, 11 aa, Artificial Sequence
  Synthetic polypeptide
GDYGYDDPLDY

SEQ ID NO: 48 - protein, 9 aa, Artificial Sequence
  Synthetic polypeptide
DLGAAASDY

SEQ ID NO: 49 - protein, 11 aa, Artificial Sequence
  Synthetic polypeptide
NRRDEYYAMDY

SEQ ID NO: 50 - protein, 13 aa, Artificial Sequence
  Synthetic polypeptide
ARGDYGYDDPLDY

SEQ ID NO: 51 - protein, 11 aa, Artificial Sequence
  Synthetic polypeptide
NRRDEYYAMDY

SEQ ID NO: 52 - protein, 11 aa, Artificial Sequence
  Synthetic polypeptide
ARDLGAAASDY

SEQ ID NO: 53 - protein, 11 aa, Artificial Sequence
  Synthetic polypeptide
GYFDWVDYFDY

SEQ ID NO: 54 - protein, 12 aa, Artificial Sequence
  Synthetic polypeptide
NGDSDYYSGMDY

SEQ ID NO: 55 - protein, 4 aa, Artificial Sequence
  Synthetic polypeptide
PFAY

SEQ ID NO: 56 - protein, 6 aa, Artificial Sequence
  Synthetic polypeptide
ARPGDY

SEQ ID NO: 57 - protein, 9 aa, Artificial Sequence
  Synthetic polypeptide
DQLGDAFDI

SEQ ID NO: 58 - protein, 9 aa, Artificial Sequence
  Synthetic polypeptide
DSAAHGMDV

SEQ ID NO: 59 - protein, 9 aa, Artificial Sequence
  Synthetic polypeptide
ASAAHGMDV

SEQ ID NO: 60 - protein, 9 aa, Artificial Sequence
  Synthetic polypeptide
DSAAHGMDV

SEQ ID NO: 61 - protein, 9 aa, Artificial Sequence
  Synthetic polypeptide
DSAAHGMDV

SEQ ID NO: 62 - protein, 9 aa, Artificial Sequence
  Synthetic polypeptide
DSAAHGMDV

SEQ ID NO: 63 - protein, 8 aa, Artificial Sequence
  Synthetic polypeptide
DSGGLFDY

SEQ ID NO: 64 - protein, 10 aa, Artificial Sequence
  Synthetic polypeptide
DQSIIETFDY

SEQ ID NO: 65 - protein, 9 aa, Artificial Sequence
  Synthetic polypeptide
EGGRDAFDI

SEQ ID NO: 66 - protein, 9 aa, Artificial Sequence
  Synthetic polypeptide
EIAVALFDY

SEQ ID NO: 67 - protein, 15 aa, Artificial Sequence
  Synthetic polypeptide
RASESVDNFGISFMN

SEQ ID NO: 68 - protein, 16 aa, Artificial Sequence
  Synthetic polypeptide
RSSKSLLHTNGNTYLH

SEQ ID NO: 69 - protein, 16 aa, Artificial Sequence
  Synthetic polypeptide
RSSKSLLHTNQNTYLH

SEQ ID NO: 70 - protein, 11 aa, Artificial Sequence
  Synthetic polypeptide
RASQGINSALA

SEQ ID NO: 71 - protein, 10 aa, Artificial Sequence
  Synthetic polypeptide
ESVDNFGISF

SEQ ID NO: 72 - protein, 11 aa, Artificial Sequence
  Synthetic polypeptide
KSLLHTNGNTY

SEQ ID NO: 73 - protein, 10 aa, Artificial Sequence
  Synthetic polypeptide
ESVDNFGISF

SEQ ID NO: 74 - protein, 6 aa, Artificial Sequence
  Synthetic polypeptide
QGINSA

SEQ ID NO: 75 - protein, 11 aa, Artificial Sequence
  Synthetic polypeptide
KSLLHTNGNTY

SEQ ID NO: 76 - protein, 11 aa, Artificial Sequence
  Synthetic polypeptide
RASQGISSWLA

SEQ ID NO: 77 - protein, 11 aa, Artificial Sequence
  Synthetic polypeptide
RTSQSIGTNIH

SEQ ID NO: 78 - protein, 11 aa, Artificial Sequence
  Synthetic polypeptide
RASQEISGYLS

SEQ ID NO: 79 - protein, 11 aa, Artificial Sequence
  Synthetic polypeptide
RASQSVGSYVD

SEQ ID NO: 80 - protein, 11 aa, Artificial Sequence
  Synthetic polypeptide
KASQSVSSSLA

SEQ ID NO: 81 - protein, 11 aa, Artificial Sequence
  Synthetic polypeptide
RASQGISSWLA

SEQ ID NO: 82 - protein, 11 aa, Artificial Sequence
  Synthetic polypeptide
RASQGISSWLA

SEQ ID NO: 83 - protein, 11 aa, Artificial Sequence
  Synthetic polypeptide
RASQGISSWLA

SEQ ID NO: 84 - protein, 11 aa, Artificial Sequence
  Synthetic polypeptide
RASQGISSWLA

SEQ ID NO: 85 - protein, 11 aa, Artificial Sequence
  Synthetic polypeptide
RASQSVSSYLA

SEQ ID NO: 86 - protein, 11 aa, Artificial Sequence
  Synthetic polypeptide
RASQSVSSYLA

SEQ ID NO: 87 - protein, 11 aa, Artificial Sequence
  Synthetic polypeptide
RASQGISSALA

SEQ ID NO: 88 - protein, 11 aa, Artificial Sequence
  Synthetic polypeptide
RASQSVSSYLA

SEQ ID NO: 89 - protein, 7 aa, Artificial Sequence
  Synthetic polypeptide
GASNQGS

SEQ ID NO: 90 - protein, 7 aa, Artificial Sequence
  Synthetic polypeptide
RMSVLAS

SEQ ID NO: 91 - protein, 7 aa, Artificial Sequence
  Synthetic polypeptide
RMSVLAS

SEQ ID NO: 92 - protein, 7 aa, Artificial Sequence
  Synthetic polypeptide
DASSLES

SEQ ID NO: 93 - protein, 3 aa, Artificial Sequence
  Synthetic polypeptide
GAS

SEQ ID NO: 94 - protein, 3 aa, Artificial Sequence
  Synthetic polypeptide
RMS

SEQ ID NO: 95 - protein, 3 aa, Artificial Sequence
  Synthetic polypeptide
GAS

SEQ ID NO: 96 - protein, 3 aa, Artificial Sequence
  Synthetic polypeptide
DAS

SEQ ID NO: 97 - protein, 3 aa, Artificial Sequence
  Synthetic polypeptide
RMS

SEQ ID NO: 98 - protein, 7 aa, Artificial Sequence
  Synthetic polypeptide
AASSLQS

SEQ ID NO: 99 - protein, 7 aa, Artificial Sequence
  Synthetic polypeptide
NVSESIS

SEQ ID NO: 100 - protein, 7 aa, Artificial Sequence
  Synthetic polypeptide
YVSESIS

SEQ ID NO: 101 - protein, 7 aa, Artificial Sequence
  Synthetic polypeptide
YASESIS

SEQ ID NO: 102 - protein, 7 aa, Artificial Sequence
  Synthetic polypeptide
ATSALDS

SEQ ID NO: 103 - protein, 7 aa, Artificial Sequence
  Synthetic polypeptide
GASTRYT

SEQ ID NO: 104 - protein, 7 aa, Artificial Sequence
  Synthetic polypeptide
DASNRAT

SEQ ID NO: 105 - protein, 7 aa, Artificial Sequence
  Synthetic polypeptide
AASSLQS

SEQ ID NO: 106 - protein, 7 aa, Artificial Sequence
  Synthetic polypeptide
AASSLQS

SEQ ID NO: 107 - protein, 7 aa, Artificial Sequence
  Synthetic polypeptide
AASSLQS

SEQ ID NO: 108 - protein, 7 aa, Artificial Sequence
  Synthetic polypeptide
AASSLQS

SEQ ID NO: 109 - protein, 7 aa, Artificial Sequence
  Synthetic polypeptide
DASNRAT

SEQ ID NO: 110 - protein, 7 aa, Artificial Sequence
  Synthetic polypeptide
DASNRAT

SEQ ID NO: 111 - protein, 7 aa, Artificial Sequence
  Synthetic polypeptide
DASSLES

SEQ ID NO: 112 - protein, 7 aa, Artificial Sequence
  Synthetic polypeptide
DASNRAT

SEQ ID NO: 113 - protein, 9 aa, Artificial Sequence
  Synthetic polypeptide
QQSKEVPWT

SEQ ID NO: 114 - protein, 9 aa, Artificial Sequence
  Synthetic polypeptide
MQHLEYPLT

SEQ ID NO: 115 - protein, 9 aa, Artificial Sequence
  Synthetic polypeptide
MQHLEYPLT

SEQ ID NO: 116 - protein, 9 aa, Artificial Sequence
  Synthetic polypeptide
QQFNSYPHT

SEQ ID NO: 117 - protein, 9 aa, Artificial Sequence
  Synthetic polypeptide
QQSKEVPWT

SEQ ID NO: 118 - protein, 9 aa, Artificial Sequence
  Synthetic polypeptide
MQHLEYPLT

SEQ ID NO: 119 - protein, 9 aa, Artificial Sequence
  Synthetic polypeptide
QQSKEVPWT

SEQ ID NO: 120 - protein, 9 aa, Artificial Sequence
  Synthetic polypeptide
QQFNSYPHT

SEQ ID NO: 121 - protein, 9 aa, Artificial Sequence
  Synthetic polypeptide
MQHLEYPLT

SEQ ID NO: 122 - protein, 9 aa, Artificial Sequence
  Synthetic polypeptide
QQYNSYPPT

SEQ ID NO: 123 - protein, 9 aa, Artificial Sequence
  Synthetic polypeptide
QQSNTWPFT

SEQ ID NO: 124 - protein, 9 aa, Artificial Sequence
  Synthetic polypeptide
LQYANYPYT

SEQ ID NO: 125 - protein, 9 aa, Artificial Sequence
  Synthetic polypeptide
LQYNNHPYT

SEQ ID NO: 126 - protein, 10 aa, Artificial Sequence
  Synthetic polypeptide
QQRSNWPPYT

SEQ ID NO: 127 - protein, 9 aa, Artificial Sequence
  Synthetic polypeptide
QQYNSYPYT

SEQ ID NO: 128 - protein, 9 aa, Artificial Sequence
  Synthetic polypeptide
QQYNSYPYT

SEQ ID NO: 129 - protein, 9 aa, Artificial Sequence
  Synthetic polypeptide
QQYNSYPYT

SEQ ID NO: 130 - protein, 9 aa, Artificial Sequence
  Synthetic polypeptide
QQYNSYPYT

SEQ ID NO: 131 - protein, 10 aa, Artificial Sequence
  Synthetic polypeptide
QQRSNWPHLT

SEQ ID NO: 132 - protein, 9 aa, Artificial Sequence
  Synthetic polypeptide
QQRSNWGFT

SEQ ID NO: 133 - protein, 9 aa, Artificial Sequence
  Synthetic polypeptide
QQFNSYPHT

SEQ ID NO: 134 - protein, 10 aa, Artificial Sequence
  Synthetic polypeptide
QQRSSWPPYT

SEQ ID NO: 135 - protein, 7 aa, Artificial Sequence
  Synthetic polypeptide
TSGMGVG

SEQ ID NO: 136 - protein, 8 aa, Artificial Sequence
  Synthetic polypeptide
GFTFSNYG

SEQ ID NO: 137 - protein, 8 aa, Artificial Sequence
  Synthetic polypeptide
GFTFSSYG

SEQ ID NO: 138 - protein, 6 aa, Artificial Sequence
  Synthetic polypeptide
SSNWWT

SEQ ID NO: 139 - protein, 7 aa, Artificial Sequence
  Synthetic polypeptide
FNNYYMD

SEQ ID NO: 140 - protein, 5 aa, Artificial Sequence
  Synthetic polypeptide
NYYIH

SEQ ID NO: 141 - protein, 5 aa, Artificial Sequence
  Synthetic polypeptide
DTYMH

SEQ ID NO: 142 - protein, 16 aa, Artificial Sequence
  Synthetic polypeptide
HIWWDDDKRYNPALKS

SEQ ID NO: 143 - protein, 8 aa, Artificial Sequence
  Synthetic polypeptide
IYYSGGST

SEQ ID NO: 144 - protein, 8 aa, Artificial Sequence
  Synthetic polypeptide
VNHSGGST

SEQ ID NO: 145 - protein, 15 aa, Artificial Sequence
  Synthetic polypeptide
RISGSGGATNYNPSL

SEQ ID NO: 146 - protein, 15 aa, Artificial Sequence
  Synthetic polypeptide
RISSSGDPTWYADSV

SEQ ID NO: 147 - protein, 17 aa, Artificial Sequence
  Synthetic polypeptide
WIFPGNFKTEYNEKFKG

SEQ ID NO: 148 - protein, 17 aa, Artificial Sequence
  Synthetic polypeptide
RIDPANGNTKYDPKFQG

SEQ ID NO: 149 - protein, 8 aa, Artificial Sequence
  Synthetic polypeptide
INPAWFAY

SEQ ID NO: 150 - protein, 7 aa, Artificial Sequence
  Synthetic polypeptide
ARESIDY

SEQ ID NO: 151 - protein, 8 aa, Artificial Sequence
  Synthetic polypeptide
ARVGSFDF

SEQ ID NO: 152 - protein, 12 aa, Artificial Sequence
  Synthetic polypeptide
DWAQIAGTTLGF

SEQ ID NO: 153 - protein, 7 aa, Artificial Sequence
  Synthetic polypeptide
LTTGSDS

SEQ ID NO: 154 - protein, 7 aa, Artificial Sequence
  Synthetic polypeptide
YGYAVDY

SEQ ID NO: 155 - protein, 8 aa, Artificial Sequence
  Synthetic polypeptide
YYGIYVDY

SEQ ID NO: 156 - protein, 15 aa, Artificial Sequence
  Synthetic polypeptide
KASQSVDFDGDSFMN

SEQ ID NO: 157 - protein, 14 aa, Artificial Sequence
  Synthetic polypeptide
TGTSDDVGGYNYVS

SEQ ID NO: 158 - protein, 11 aa, Artificial Sequence
  Synthetic polypeptide
RASQDIRYYLN

SEQ ID NO: 159 - protein, 9 aa, Artificial Sequence
  Synthetic polypeptide
KASQDVSTA

SEQ ID NO: 160 - protein, 16 aa, Artificial Sequence
  Synthetic polypeptide
KSSQSLLDSDGKTYLN

SEQ ID NO: 161 - protein, 7 aa, Artificial Sequence
  Synthetic polypeptide
TTSNLES

SEQ ID NO: 162 - protein, 7 aa, Artificial Sequence
  Synthetic polypeptide
DVAKRAS

SEQ ID NO: 163 - protein, 7 aa, Artificial Sequence
  Synthetic polypeptide
VASSLQS

SEQ ID NO: 164 - protein, 7 aa, Artificial Sequence
  Synthetic polypeptide
SASYRYT

SEQ ID NO: 165 - protein, 7 aa, Artificial Sequence
  Synthetic polypeptide
LVSKLDS

SEQ ID NO: 166 - protein, 9 aa, Artificial Sequence
  Synthetic polypeptide
QQSNEDPYT

SEQ ID NO: 167 - protein, 10 aa, Artificial Sequence
  Synthetic polypeptide
CSYTTSSTLL

SEQ ID NO: 168 - protein, 9 aa, Artificial Sequence
  Synthetic polypeptide
LQVYSTPRT

SEQ ID NO: 169 - protein, 9 aa, Artificial Sequence
  Synthetic polypeptide
QQHYITPLT

SEQ ID NO: 170 - protein, 9 aa, Artificial Sequence
  Synthetic polypeptide
WQDTHFPHV

SEQ ID NO: 171 - protein, 5 aa, Artificial Sequence
  Synthetic polypeptide
SYGIS

SEQ ID NO: 172 - protein, 12 aa, Artificial Sequence
  Synthetic polypeptide
GFSLSTYGVGVG

SEQ ID NO: 173 - protein, 16 aa, Artificial Sequence
  Synthetic polypeptide
EIYPRSGNTYYNEKFK

SEQ ID NO: 174 - protein, 16 aa, Artificial Sequence
  Synthetic polypeptide
NIWWDDDKRYNPSLEN

SEQ ID NO: 175 - protein, 9 aa, Artificial Sequence
  Synthetic polypeptide
STTVSPADF

SEQ ID NO: 176 - protein, 9 aa, Artificial Sequence
  Synthetic polypeptide
STTVSPPPI

SEQ ID NO: 177 - protein, 9 aa, Artificial Sequence
  Synthetic polypeptide
STTVSPPAH

SEQ ID NO: 178 - protein, 9 aa, Artificial Sequence
  Synthetic polypeptide
STTVAPPRL

SEQ ID NO: 179 - protein, 9 aa, Artificial Sequence
  Synthetic polypeptide
STTVHPDRN

SEQ ID NO: 180 - protein, 9 aa, Artificial Sequence
  Synthetic polypeptide
STTVSPPAL

SEQ ID NO: 181 - protein, 9 aa, Artificial Sequence
  Synthetic polypeptide
STTVHPDHN

SEQ ID NO: 182 - protein, 9 aa, Artificial Sequence
  Synthetic polypeptide
STTVSPPHL

SEQ ID NO: 183 - protein, 9 aa, Artificial Sequence
  Synthetic polypeptide
STTVAPPPL

SEQ ID NO: 184 - protein, 9 aa, Artificial Sequence
  Synthetic polypeptide
STTVSPPHL

SEQ ID NO: 185 - protein, 9 aa, Artificial Sequence
  Synthetic polypeptide
STTVAPPGH

SEQ ID NO: 186 - protein, 9 aa, Artificial Sequence
  Synthetic polypeptide
STTVSPPRV

SEQ ID NO: 187 - protein, 9 aa, Artificial Sequence
  Synthetic polypeptide
STTVSPPPL

SEQ ID NO: 188 - protein, 9 aa, Artificial Sequence
  Synthetic polypeptide
STTVAPPAH

SEQ ID NO: 189 - protein, 9 aa, Artificial Sequence
  Synthetic polypeptide
STTVRPPGI

SEQ ID NO: 190 - protein, 9 aa, Artificial Sequence
  Synthetic polypeptide
STTVSAPGV

SEQ ID NO: 191 - protein, 13 aa, Artificial Sequence
  Synthetic polypeptide
TPAYYGSHPPFDY

SEQ ID NO: 192 - protein, 11 aa, Artificial Sequence
  Synthetic polypeptide
KASDHINNWLA

SEQ ID NO: 193 - protein, 11 aa, Artificial Sequence
  Synthetic polypeptide
RTSEDIYTNLA

SEQ ID NO: 194 - protein, 7 aa, Artificial Sequence
  Synthetic polypeptide
GATSLET

SEQ ID NO: 195 - protein, 7 aa, Artificial Sequence
  Synthetic polypeptide
VAKTLQD

SEQ ID NO: 196 - protein, 7 aa, Artificial Sequence
  Synthetic polypeptide
VAKTLQE

SEQ ID NO: 197 - protein, 9 aa, Artificial Sequence
  Synthetic polypeptide
NTYGNNPHT

SEQ ID NO: 198 - protein, 9 aa, Artificial Sequence
  Synthetic polypeptide
HQYYNTPYT

SEQ ID NO: 199 - protein, 9 aa, Artificial Sequence
  Synthetic polypeptide
HQYYSTPYT

SEQ ID NO: 200 - protein, 9 aa, Artificial Sequence
  Synthetic polypeptide
QQYYSTPYT

SEQ ID NO: 201 - protein, 9 aa, Artificial Sequence
  Synthetic polypeptide
LQGFKFPWT

SEQ ID NO: 202 - protein, 9 aa, Artificial Sequence
  Synthetic polypeptide
GGSGGGGSG

SEQ ID NO: 203 - protein, 5 aa, Artificial Sequence
  Synthetic polypeptide
TVAAP

SEQ ID NO: 204 - protein, 13 aa, Artificial Sequence
  Synthetic polypeptide
GGSGGGGSGGGGS

SEQ ID NO: 205 - protein, 12 aa, Artificial Sequence
  Synthetic polypeptide
TVAAPSVFIFPP

SEQ ID NO: 206 - protein, 16 aa, Artificial Sequence
  Synthetic polypeptide
AKTTPKLEEGEFSEAR

SEQ ID NO: 207 - protein, 17 aa, Artificial Sequence
  Synthetic polypeptide
AKTTPKLEEGEFSEARV

SEQ ID NO: 208 - protein, 9 aa, Artificial Sequence
  Synthetic polypeptide
AKTTPKLGG

SEQ ID NO: 209 - protein, 10 aa, Artificial Sequence
  Synthetic polypeptide
SAKTTPKLGG

SEQ ID NO: 210 - protein, 6 aa, Artificial Sequence
  Synthetic polypeptide
SAKTTP

SEQ ID NO: 211 - protein, 6 aa, Artificial Sequence
  Synthetic polypeptide
RADAAP

SEQ ID NO: 212 - protein, 9 aa, Artificial Sequence
  Synthetic polypeptide
RADAAPTVS

SEQ ID NO: 213 - protein, 12 aa, Artificial Sequence
  Synthetic polypeptide
RADAAAAGGPGS

SEQ ID NO: 214 - protein, 27 aa, Artificial Sequence
  Synthetic polypeptide
RADAAAAGGGGSGGGGSGGGGSGGGGS

SEQ ID NO: 215 - protein, 18 aa, Artificial Sequence
  Synthetic polypeptide
SAKTTPKLEEGEFSEARV

SEQ ID NO: 216 - protein, 5 aa, Artificial Sequence
  Synthetic polypeptide
ADAAP

SEQ ID NO: 217 - protein, 12 aa, Artificial Sequence
  Synthetic polypeptide
ADAAPTVSIFPP

SEQ ID NO: 218 - protein, 6 aa, Artificial Sequence
  Synthetic polypeptide
QPKAAP

SEQ ID NO: 219 - protein, 13 aa, Artificial Sequence
  Synthetic polypeptide
QPKAAPSVTLFPP

SEQ ID NO: 220 - protein, 6 aa, Artificial Sequence
  Synthetic polypeptide
AKTTPP

SEQ ID NO: 221 - protein, 13 aa, Artificial Sequence
  Synthetic polypeptide
AKTTPPSVTPLAP

SEQ ID NO: 222 - protein, 6 aa, Artificial Sequence
  Synthetic polypeptide
AKTTAP

SEQ ID NO: 223 - protein, 13 aa, Artificial Sequence
  Synthetic polypeptide
AKTTAPSVYPLAP

SEQ ID NO: 224 - protein, 6 aa, Artificial Sequence
  Synthetic polypeptide
ASTKGP

SEQ ID NO: 225 - protein, 13 aa, Artificial Sequence
  Synthetic polypeptide
ASTKGPSVFPLAP

SEQ ID NO: 226 - protein, 14 aa, Artificial Sequence
  Synthetic polypeptide
GGGSGGGGSGGGGS

SEQ ID NO: 227 - protein, 15 aa, Artificial Sequence
  Synthetic polypeptide
GENKVEYAPALMALS

SEQ ID NO: 228 - protein, 15 aa, Artificial Sequence
  Synthetic polypeptide
GPAKELTPLKEAKVS

SEQ ID NO: 229 - protein, 15 aa, Artificial Sequence
  Synthetic polypeptide
GHEAAAVMQVQYPAS

SEQ ID NO: 230 - protein, 4 aa, Homo sapiens
GPYT

SEQ ID NO: 231 - protein, 6 aa, Homo sapiens
ALNGEE

SEQ ID NO: 232 - protein, 8 aa, Homo sapiens
DWPEALAI

SEQ ID NO: 233 - protein, 17 aa, Homo sapiens
VKVTFFQNGKSQKFSRL

SEQ ID NO: 234 - protein, 17 aa, Homo sapiens
VKVTFFQNGKSQKFSHL

SEQ ID NO: 235 - protein, 4 aa, Homo sapiens
NIGY

SEQ ID NO: 236 - protein, 19 aa, Homo sapiens
FFQNGKSKKFSRSDPNFSI

SEQ ID NO: 237 - protein, 17 aa, Homo sapiens
HKVTYLQNGKDRKYFHH

SEQ ID NO: 238 - protein, 4 aa, Homo sapiens
LVGS

SEQ ID NO: 239 - protein, 4 aa, Homo sapiens
LFGS

SEQ ID NO: 240 - protein, 444 aa, Artificial Sequence
  Synthetic polypeptide
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 241 - protein, 214 aa, Artificial Sequence
  Synthetic polypeptide
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 447 aa, Artificial Sequence
  Synthetic polypeptide
QVHLVESGGGVVPGRSLRLSCAASGFTFSSYGMHWVRQAPGKGLEWVAVIWYDGSNYYYTDSVKGRFTISRDNSKNTLYLQMNSLRAEDTAVYYCARDLGAAASDYWGQGTLVTVSSASTKGPSVFPLAPSSLSTSGGTAALGCLVKDYFPEPVTVSWNSGALTSGVHTFPAVLQSSGLYSLSSVVTVPSSSLGTQTYTCNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQFASTFRVVSVLTVLHQDWLNGKEYKCKVSNKGLPAPIEKTISKAKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 243 - protein, 213 aa, Artificial Sequence
  Synthetic polypeptide
AIQLTQSPSSLSASVGDRVTITCRASQGINSALAWYQQKPGKAPKLLIYDASSLESGVPSRFSGSGSGTDFTLTISSLQPEDFATYYCQQFNSYPHTFGQGTKLEIKRTVAAPSVFIFPPSDEQLKGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - DNA, 22 bp, Artificial Sequence
  Synthetic oligonucleotide
tcttgacgctgggacctaggcg

SEQ ID NO: 245 - DNA, 22 bp, Artificial Sequence
  Synthetic oligonucleotide
gggcttgacttcatgtcccccg

SEQ ID NO: 246 - DNA, 22 bp, Artificial Sequence
  Synthetic oligonucleotide
actaccaagccagtgctgcgaa

SEQ ID NO: 247 - DNA, 22 bp, Artificial Sequence
  Synthetic oligonucleotide
atcacaggcgaagtccaatccg

SEQ ID NO: 248 - DNA, 21 bp, Artificial Sequence
  Synthetic oligonucleotide
gagagctgcagggccctttgc

SEQ ID NO: 249 - DNA, 25 bp, Artificial Sequence
  Synthetic oligonucleotide
ctccctggtttctcttcccaagacc

SEQ ID NO: 250 - DNA, 20 bp, Artificial Sequence
  Synthetic oligonucleotide
ccctcctggccaacggcatg

SEQ ID NO: 251 - DNA, 20 bp, Artificial Sequence
  Synthetic oligonucleotide
tcggggcagccttgtccctt

SEQ ID NO: 252 - DNA, 25 bp, Artificial Sequence
  Synthetic oligonucleotide
tgcaagagacttccatccagttgcc

SEQ ID NO: 253 - DNA, 23 bp, Artificial Sequence
  Synthetic oligonucleotide
tgtgaagtagggaaggccgtggt

SEQ ID NO: 254 - DNA, 22 bp, Artificial Sequence
  Synthetic oligonucleotide
acgagagttgcctggctactag

SEQ ID NO: 255 - DNA, 23 bp, Artificial Sequence
  Synthetic oligonucleotide
cctcatagatgctaccaaggcac

SEQ ID NO: 256 - DNA, 22 bp, Artificial Sequence
  Synthetic oligonucleotide
cccctgactctcgggcagtgac

SEQ ID NO: 257 - DNA, 21 bp, Artificial Sequence
  Synthetic oligonucleotide
tctgctgccgtgcttccaacg

SEQ ID NO: 258 - DNA, 20 bp, Artificial Sequence
  Synthetic oligonucleotide
gacagtcagccgcatcttct

SEQ ID NO: 259 - DNA, 20 bp, Artificial Sequence
  Synthetic oligonucleotide
gcgcccaatacgaccaaatc

SEQ ID NO: 260 - DNA, 35 bp, Artificial Sequence
  Synthetic oligonucleotide
atcccagaaattctcccgtttggatcccaccttct

SEQ ID NO: 261 - DNA, 35 bp, Artificial Sequence
  Synthetic oligonucleotide
agaaggtgggatccaaacgggagaatttctgggat

(19) United States
(12) Patent Application Publication
(10) Pub. No.: US 2021/0139582 A1
(43) Pub. Date: May 13, 2021

(54) THERAPEUTIC FCRN-BASED BISPECIFIC MONOCLONAL ANTIBODIES

(71) Applicant: THE BRIGHAM AND WOMEN'S HOSPITAL, INC., Boston, MA (US)

(72) Inventors: Richard S. BLUMBERG, Weston, MA (US); Michal PYZIK, Watertown, MA (US); Jonathan HUBBARD, Newton Highlands, MA (US); Amit GANDHI, Billerica, MA (US)

(73) Assignee: THE BRIGHAM AND WOMEN'S HOSPITAL, INC., Boston, MA (US)

(21) Appl. No.: 16/969,425

(22) PCT Filed: Feb. 13, 2019
(86) PCT No.: PCT/US2019/017880

§ 371 Date: Aug. 12, 2020

Related U.S. Application Data
(60) Provisional application No. 62/629,749, filed on Feb. 13, 2018.

(51) Int. Cl. C07K 16/28; A61P 37/02

(52) U.S. Cl. CPC C07K 16/283; A61P 37/02; C07K 2317/31; A61K 2039/505; C07K 2317/62; C07K 2317/24; C07K 2317/76

(57) ABSTRACT

The technology described herein is directed to immunotherapy agents for autoimmune disease, cancer, or allergy. In some embodiments, the immunotherapy agent comprises a bispecific antibody construct that specifically binds FcRn and a Type I or Type II Fcγ T receptor. In some embodiments the bispecific antibody construct is a DvD-Ig construct. Also described herein are methods for treating autoimmune disease, cancer, or allergy, comprising administering an effective amount of a bispecific antibody construct to patient in need thereof.

### CROSS-REFERENCE TO RELATED APPLICATIONS

This application is a 35 U.S.C. § 371 National Phase Entry application of International Patent Application No. PCT/US2019/017880 filed on Feb. 13, 2019 which claims benefit under 35 U.S.C. § 119(e) of U.S. Provisional Application No. 62/629,749 filed Feb. 13, 2018, the contents of which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Feb. 13, 2019, is named 043214-091690WOPT-SEQ.txt and is 63,812 bytes in size.

### TECHNICAL FIELD

The technology described herein relates to immunotherapy.

### BACKGROUND

It is well known that receptors that bind the constant domains of antibodies play important roles in both immune-related signaling and promotion of extended circulating half-lives of antibody molecules. For example, the so-called neonatal Fc receptor, FcRn, binds IgG and participates in intracellular trafficking of the antibody. Originally identified as a receptor important in passive neonatal immunity, mediating transfer of maternal IgG across the placenta or neonatal intestinal walls, FcRn was subsequently found to function throughout adult life, being expressed in various tissues, such as the epithelium of the lung and liver, vascular endothelium, monocytes, macrophages and dendritic cells.

FcRn was first isolated from rodent gut as a heterodimer between a 12 kDa and a 40-50 kDa protein (Rodewald & Kraehenbuhl 1984, J. Cell. Biol. 99(1 Pt2): 159s-154s; Simister & Rees, 1985, Eur. J. Immunol. 15:733-738) and was cloned in 1989 (Simister & Mostov, 1989, Nature 337:184-187). Cloning and subsequent crystallization of FcRn revealed it to have an approximately 50 kDa major histocompatibility complex (MHC) class I-like heavy chain in non-covalent association with a 12 kDa β2-microglobulin light chain (Raghavan et al., 1993, Biochemistry 32:8654-8660; Huber et al., 1993, J. Mol. Biol. 230:1077-1083).

FcRn resides primarily in the early acidic endosomes where it binds to the Fc region of IgG in a pH-dependent manner, with micro- to nanomolar affinity at pH 6.5, while binding of FcRn to Fc at physiological pH is negligible. The bulk of FcRn is present in endosomes in most cells, and the interaction between FcRn and its IgG Fc ligands occurs within that acidic environment. In some cells, such as hematopoietic cells, significant levels of FcRn can be detected on the cell surface in addition to intracellular expression (Zhu et al., 2001, J. Immunol. 166:3266-3276). In this case, when the extracellular milieu is acidic, as in the case of neoplastic or infectious conditions, it is possible that FcRn can bind to IgG on the cell surface of these cell types. FcRn regulates serum IgG concentrations by binding to and protecting endocytosed monomeric IgG from degradation in the lysosomal compartment, and transporting the IgG to the cell surface for release at neutral extracellular pH. Through this mechanism, FcRn is responsible for the long serum half-life of IgG, since IgG that is not bound by FcRn enters the lysosomal pathway and is degraded.

FcRn-deficient mice are more resistant to autoimmune diseases caused by pathogenic IgG autoantibodies because they are unable to maintain high concentrations of pathogenic serum IgG (Christianson et al., 1996, J. Immunol. 156:4932-4939; Ghetie et al., 1996, Eur. J. Immunol. 26:690-696; Israel et al., 1996, Immunol. 89:573-578). Administration of antibodies engineered to have modified Fc regions that bind with higher affinity to FcRn was found to ameliorate disease in a murine arthritis model (Patel et al., 2011, J. Immunol. 187:1015-1022). High dose administration of IgG in a number of autoimmune diseases has a palliative effect that can be explained at least partially by saturation of FcRn-mediated protection of IgG, shortening the half-life of pathogenic IgG (Jin & Balthasar, 2005, Hum. Immunol. 66:403-410; Akilesh et al., 2004, J. Clin. Invest. 113:1328-1333; Li et al., 2005, J. Clin. Invest. 115:3440-3450). Accordingly, specific blockade of FcRn-IgG interaction can be used to promote degradation of pathogenic IgG antibodies, for example to treat IgG mediated autoimmune diseases and to clear therapeutic antibodies from serum after administration. For example, in a rat model of experimentally-induced autoimmune myasthenia gravis, treatment with an FcRn heavy-chain specific monoclonal antibody resulted in a reduction of serum IgG concentration and a decrease in severity of the disease (Liu et al., 2007, J. Immunol. 178:5390-5398).

An absence of FcRn in hematopoietic cells is associated with more rapid clearance of IgG containing immune complexes from the bloodstream (Qiao et al., 2008, Proc. Natl. Acad. Sci. USA 105: 9337-9342). This indicates that specific blockade of FcRn-IgG interactions will also promote the clearance of IgG containing immune complexes from the circulation.

FcRn regulates the movement of IgG, and any bound cargo, between different compartments of the body via transcytosis across polarized cells. This process plays an important role in mucosal protection from infection, e.g., in the gastrointestinal tract. FcRn transports IgG across the epithelial cell barrier of the intestines and into the lumen. After IgG binds antigen in the lumen, the IgG/antigen complex is transported back through the barrier by FcRn into the lamina propria, allowing for processing of the IgG/antigen complex by dendritic cells and presentation of antigen to CD4⁺ T cells in regional lymph nodes.

FcRn also plays an important role in MHC class II antigen presentation and MHC class I cross-presentation of IgG-complexed antigen. When antigen is presented as an IgG-containing immune complex (IC), dendritic cells that are CD8-CD11b⁺CD11c⁺ (inflammatory dendritic cells) display significant cross-presentation at low antigen doses in a pathway that is highly dependent upon FcRn expression. This pathway involves the internalization of the ICs by Fc7 receptors into an acidic endosome in an antigen-presenting cell (APC). Antigen from the internalized ICs is directed to cellular compartments via an FcRn-dependent mechanism, where the antigen is processed to peptides compatible with loading onto MHC molecules for display (Baker et al., 2011, Proc. Natl. Acad. Sci. USA 108:9927-9932; Christianson et al., 2012, mAbs vol. 4, page 208, Introduction). Thus, FcRn in DCs enhances MHC II antigen presentation and induces proliferation of antigen-specific CD4⁺ T-cells as well as exhibiting a fundamental role in antigen presentation to CD8⁺ T cells (cytotoxic T cells). This latter CD8⁺ T cell-pathway is called cross-presentation and involves the crossover of extracellular antigens into an MHC class I-dependent pathway.

Blockade of FcRn-Ig IC interaction inhibits antigen presentation of IC and subsequent T cell activation stimulated by immune-associated antigen presentation. Interactions with IgG IC in APCs such as DCs also promote secretion of inflammatory cytokines such as IL-12, IFNγ, and TNFα. Thus, blockade of FcRn-Ig IC interaction is useful to inhibit production of inflammatory cytokines by innate immune cells and antigen activated T cells.

While blockade of FcRn-Ig IC interaction is therapeutically useful in the treatment of autoimmune disease, and particularly autoimmune disease mediated by IgG, such blockade tends to indiscriminately reduce serum IgG, affecting the half-life and serum concentration of both immune complex IgGs and monomeric IgGs.

All publications cited herein are incorporated by reference in their entirety to the same extent as if each individual publication or patent application was specifically and individually indicated to be incorporated by reference. The following description includes information that may be useful in understanding the present claimed invention. It is not an admission that any of the information provided herein is prior art or relevant to the present claims, or that any publication specifically or implicitly referenced is prior art.

### SUMMARY

The technology described herein is based, in part, upon the discovery that FcRn, IgG and Type I and Type II Fc receptors form a tripartite or ternary complex in vivo, and that this complex is specific for immune complex IgG. The discovery that this ternary complex includes immune complex IgG, but not monomeric IgG, provides a target for the blockade of FcRn-mediated effects on IgG antibody concentration, including autoimmune IgG concentration that is selective for immune complex IgG, thus sparing monomeric IgG from degradation and avoiding, for example, hypogammaglobulinemia that can occur when FcRn blockade is conducted by current, non-selective approaches.

The identification of a ternary FcRn:immune complex IgG:Fcγ receptor (Type I or Type II) complex also provides avenues for the treatment of, e.g., cancer or chronic infection and allergy. As described herein below in further detail, when the Fcγ receptor is an inhibitory receptor, such as CD32b, inhibition of its signaling can promote or enhance an immune response, including an anti-cancer or anti-infection immune response, e.g., in a manner analogous to the inhibition of T cell checkpoint receptors such as CTLA-4, PD-1, and TIGIT among others.

Similarly, when the Fc receptor, such as FcpR, binds IgE that mediates allergic reactions, specific inhibition of that interaction can benefit the treatment of allergies.

Described herein are approaches that specifically inhibit the ternary complex formation between FcRn, immune complex immunoglobulins and Type I or Type II Fc receptors for therapeutic benefit.

In one aspect, described herein is a composition that selectively inhibits interaction between a type I Fc receptor or a type II Fc receptor, FcRn and an immunocomplexed antibody, the composition comprising a first binding domain that specifically binds a human type I Fc receptor or a human type II Fc receptor and a second binding domain that specifically binds a human FcRn. In one embodiment, the composition is a polypeptide composition. In another embodiment, the composition comprises a nucleic acid encoding the polypeptide composition. In another embodiment, the composition comprises a cell comprising the polypeptide composition or a cell comprising a nucleic acid encoding the polypeptide composition.

In one embodiment of this and other aspects described herein, the first and/or second binding domains comprise antibody antigen binding domains. In another embodiment, the first and second binding domains each comprise an antibody antigen binding domain.

In another embodiment of this and other aspects described herein, the first and second binding domains are comprised by a human, humanized, or chimeric antibody construct.

In another embodiment of this and other aspects described herein, the first and second binding domains are comprised by a bispecific antibody construct. In another embodiment, the bispecific antibody construct comprises a first binding domain comprising the CDRs of a V_{H}/V_{L} domain pair that specifically binds a human type I Fc receptor or a human type II Fc receptor and a second binding domain comprising the CDRs of a V_{H}/V_{L} domain pair that specifically binds a human FcRn polypeptide. In another embodiment, the V_{H} of the first V_{H}/V_{L} domain pair is joined to the V_{H} of the second V_{H}/V_{L} domain pair by a linker, and the V_{L} of the first V_{H}/V_{L} domain pair is joined to the V_{L} of the second V_{H}/V_{L} domain pair by a linker. In another embodiment, the linker is a chemical linker or a polypeptide linker. In another embodiment, the linker is selected from the group consisting of GGSGGGGSG (SEQ ID NO: 202), GGSGGGGSGGGGS (SEQ ID NO: 204), TVAAP (SEQ ID NO: 203), and TVAAPSVFIFPP (SEQ ID NO: 205). In another embodiment, the linker positions the first V_{H}/V_{L} domain pair a distance of 10-100 Å away from the second V_{H}/V_{L} domain pair, such that the composition preferentially binds FcRn and FcγR that are complexed with immunocomplexed immunoglobulin. In another embodiment, the linker positions the first V_{H}/V_{L} domain pair a distance of about 41 Å away from the second V_{H}/V_{L} domain pair. In another embodiment, wherein the first V_{H}/V_{L} domain pair is on the amino terminus of the bispecific antibody construct or the second V_{H}/V_{L} domain pair on the amino terminus of the bispecific antibody construct.

In another embodiment of this and other aspects described herein, the bispecific antibody construct is selected from the group consisting of a tandem scFv (taFv or scFv₂), diabody, dAb₂A/HH₂, knob-into-holes bispecific derivative, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)2-scFv₂, scDB-Fc, scDb-CH₃, Db-Fc, scFv₂-H/L, DVD-Ig, tandAb, scFv-dhlx-scFv, dAb2-IgG, dAb-IgG, or dAb-Fc-dAb construct. In another embodiment, the bispecific antibody construct is a diabody or a tribody. In another embodiment, the bispecific antibody construct comprises a DvD-Ig construct.

In another embodiment of this and other aspects described herein, the bispecific antibody construct is bivalent, trivalent, or tetravalent.

In another embodiment of this and other aspects described herein, the V_{H}/V_{L} domain pairs are fused to a non-immunoglobulin scaffold.

In another embodiment of this and other aspects described herein, a bispecific antibody construct comprises an immunoglobulin constant region. In another embodiment, the constant region is selected from the group consisting of IgG, IgA, IgD, IgE and IgM immunoglobulin constant regions. In another embodiment, the constant region is selected from the group consisting of IgG1, IgG2, IgG3 and IgG4 immunoglobulin constant regions. In another embodiment, the immunoglobulin constant region comprises an ΔE294 mutation, an M428L mutation, an N343S mutation or any combination thereof, wherein the mutation increases circulating half-life of the immunoglobulin. In another embodiment, the immunoglobulin constant region comprises a C_{H}3 C-terminal lysine deletion (ΔK445) (Lys0) and or an S226P mutation, wherein the mutation stabilizes the immunoglobulin hinge region. In another embodiment, the bispecific antibody construct comprises an immunoglobulin light chain. In another embodiment, the immunoglobulin light chain comprises a kappa or lambda light chain immunoglobulin polypeptide.

In another embodiment of this and other aspects described herein, for a bispecific antibody construct in which the first binding domain comprises the CDRs of a V_{H}/V_{L} domain pair that specifically binds a human type I Fc receptor or a human type II Fc receptor and a second binding domain comprising the CDRs of a V_{H}/V_{L} domain pair that specifically binds a human FcRn polypeptide, the first V_{H}/V_{L} domain pair specifically binds a type I Fc receptor selected from the group consisting of CD32, CD32a, CD32b, CD32c, CD32a^{H}, CD32a^{R}, CD16, CD16a, CD16a^{V158}, CD16a^{F158}, and CD16b. In another embodiment, the first V_{H}/V_{L} domain pair specifically binds a type II Fc receptor comprising CD23 or DC-SIGN.

In another embodiment of this and other aspects described herein, the V_{H}/V_{L} domain pair that specifically binds CD32a binds an epitope or portion of a CD32a epitope selected from the group consisting of VKVTFFQNGKSQKFSRL (SEQ ID NO: 233), VKVTFFQNGKSQKFSHL (SEQ ID NO: 234), and NIGY (SEQ ID NO: 235).

In another embodiment of this and other aspects described herein, the V_{H}/V_{L} domain pair that specifically binds CD32b binds an epitope or portion of a CD32b epitope comprising FFQNGKSKKFSRSDPNFSI (SEQ ID NO: 236).

In another embodiment of this and other aspects described herein, the V_{H}/V_{L} domain pair that specifically binds CD16a or CD16b binds an epitope or portion of a CD16a or CD16b epitope selected from the group consisting of HKVTYLQNGKDRKYFHH (SEQ ID NO: 237), LVGS (SEQ ID NO: 238), and LFGS (SEQ ID NO: 239).

In another embodiment of this and other aspects described herein, the V_{H}/V_{L} domain pair that specifically binds FcRn binds an epitope or portion of an FcRn epitope selected from the group consisting of GPYT (SEQ ID NO: 230), ALNGEE (SEQ ID NO: 231), and DWPEALAI (SEQ ID NO: 232).

In another embodiment of this and other aspects described herein, the V_{H}/V_{L} domain pair that specifically contacts CD32a comprises a V_{H} CDR1 (SEQ ID NO: 1-SEQ ID NO: 9), a V_{H} CDR2 (SEQ ID NO: 23-SEQ ID NO: 31), a V_{H} CDR3 (SEQ ID NO: 45-SEQ ID NO: 53), V_{L} CDR1 (SEQ ID NO: 67-SEQ ID NO: 76), a V_{L} CDR2 (SEQ ID NO: 89-SEQ ID NO: 98), and a V_{L} CDR3 (SEQ ID NO: 113-SEQ ID NO: 122).

In another embodiment of this and other aspects described herein, the V_{H}/V_{L} domain pair that specifically contacts CD32b comprises a V_{H} CDR1 (SEQ ID NO: 9-SEQ ID NO: 22), a V_{H} CDR2 (SEQ ID NO: 31-SEQ ID NO: 44), a V_{H} CDR3 (SEQ ID NO: 53-SEQ ID NO: 66), V_{L} CDR1 (SEQ ID NO: 76-SEQ ID NO: 88), a V_{L} CDR2 (SEQ ID NO: 98-SEQ ID NO: 112), and a V_{L} CDR3 (SEQ ID NO: 122-SEQ ID NO: 134).

In another embodiment of this and other aspects described herein, the V_{H}/V_{L} domain pair that specifically contacts CD16a or CD16b comprises a V_{H} CDR1 (SEQ ID NO: 135-SEQ ID NO: 137), a V_{H} CDR2 (SEQ ID NO: 142-SEQ ID NO: 144), a V_{H} CDR3 (SEQ ID NO: 149-SEQ ID NO: 151), V_{L} CDR1 (SEQ ID NO: 156), a V_{L} CDR2 (SEQ ID NO: 161), and a V_{L} CDR3 (SEQ ID NO: 166).

In another embodiment of this and other aspects described herein, the wherein the V_{H}/V_{L} domain pair that specifically contacts CD23 comprises a V_{H} CDR1 (SEQ ID NO: 138-SEQ ID NO: 139), a V_{H} CDR2 (SEQ ID NO: 145-SEQ ID NO: 146), a V_{H} CDR3 (SEQ ID NO: 152-SEQ ID NO: 153), V_{L} CDR1 (SEQ ID NO: 157-SEQ ID NO: 158), a V_{L} CDR2 (SEQ ID NO: 162-SEQ ID NO: 163), and a V_{L} CDR3 (SEQ ID NO: 167-SEQ ID NO: 168).

In another embodiment of this and other aspects described herein, the V_{H}/V_{L} domain pair that specifically contacts DC-SIGN comprises a V_{H} CDR1 (SEQ ID NO: 140-SEQ ID NO: 141), a V_{H} CDR2 (SEQ ID NO: 147-SEQ ID NO: 148), a V_{H} CDR3 (SEQ ID NO: 154-SEQ ID NO: 155), V_{L} CDR1 (SEQ ID NO: 159-SEQ ID NO: 160), a V_{L} CDR2 (SEQ ID NO: 164-SEQ ID NO: 165), and a V_{L} CDR3 (SEQ ID NO: 169-SEQ ID NO: 170).

In another embodiment of this and other aspects described herein, the V_{H}/V_{L} domain pair that specifically contacts FcRn comprises a V_{H} CDR1 (SEQ ID NO: 171-SEQ ID NO: 172), a V_{H} CDR2 (SEQ ID NO: 173-SEQ ID NO: 174), a V_{H} CDR3 (SEQ ID NO: 175-SEQ ID NO: 191), V_{L} CDR1 (SEQ ID NO: 192-SEQ ID NO: 193), a V_{L} CDR2 (SEQ ID NO: 194-SEQ ID NO: 196), and a V_{L} CDR3 (SEQ ID NO: 197-SEQ ID NO: 201).

In another aspect, described herein is a pharmaceutical composition comprising a composition as described herein above that selectively inhibits interaction between a type I Fc receptor or a type II Fc receptor, FcRn and an immunocomplexed antibody, and a pharmaceutically acceptable carrier.

In another aspect, described herein is a pharmaceutical composition comprising a nucleic acid encoding a polypeptide composition as described herein above that selectively inhibits interaction between a type I Fc receptor or a type II Fc receptor, FcRn and an immunocomplexed antibody, and a pharmaceutically acceptable carrier. In one embodiment, the nucleic acid is comprised by a vector. In another embodiment, the nucleic acid or vector is comprised by a cell.

In another aspect, described herein is a method for modulating the interaction between a type I Fc receptor or a type II Fc receptor, FcRn and an immunocomplexed antibody, the method comprising contacting a cell with a composition, a pharmaceutical composition, a nucleic acid, a vector or a cell as described herein above. In one embodiment, the composition does not modulate the binding of FcRn to monomeric antibodies. In another embodiment, modulating the binding of the type I Fc receptor or the type II Fc receptor and FcRn to immunocomplexed IgG occurs at a pH less than 7.

In another aspect, described herein is a method to inhibit or reduce type I Fc receptor or type II Fc receptor and FcRn interactions with an immunocomplexed antibody, the method comprising administering a therapeutically effective amount of a composition, a pharmaceutical composition, a nucleic acid, a vector or a cell as described herein above to a subject in need thereof. In one embodiment, the type I Fc receptor is selected from the group consisting of CD32, CD32a, CD32a^{H}, CD32a^{R}, CD16, CD16a, CD16a^{V158}, CD16a^{F158}, and CD16b. In another embodiment, the type II Fc receptor comprises DC-SIGN.

In another embodiment of this aspect and others described herein, the immunocomplexed antibody comprises an IgG autoantibody. In another embodiment, the level of circulating immunocomplexed IgG autoantibody is reduced. In another embodiment, the administration does not result in hypogammaglobulinemia. In another embodiment, innate and adaptive immune responses mediated by FcRn and immunocomplexed antibodies are inhibited or reduced.

In another embodiment of this aspect and others described herein, the subject has or has been diagnosed with an autoimmune disease, an IgG mediated autoimmune disease and/or an inflammatory condition. In another embodiment, the subject has or has been diagnosed with a condition selected from Kawasaki disease, Sjogren's disease, Guillain-Barre, inflammatory bowel disease (IBD), Crohn's disease, ulcerative colitis, systemic lupus erythematosus (SLE), lupus arthritis, lupus nephritis, idiopathic thrombocytopenic purpura, and/or rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behget's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia and neonatal myasthenia gravis.

In another aspect, described herein is a method to reduce the level of circulating immunocomplexed IgG autoantibodies comprising administering a therapeutically effective amount of a composition, a pharmaceutical composition, a nucleic acid, a vector, or a cell as described herein above to a subject in need thereof, wherein interaction between type I Fc receptor or type II Fc receptor and FcRn with an immunocomplexed antibody is reduced or inhibited. In one embodiment, the type I Fc receptor is selected from the group consisting of CD32, CD32a, CD32a^{H}, CD32a^{R}, CD16, CD16a, CD16a^{V158}, CD16a^{F158}, and CD16b. In another embodiment, the type II Fc receptor comprises DC-SIGN. In another embodiment, the administration does not result in hypogammaglobulinemia.

In another aspect, described herein is a method of treating an autoimmune disease, comprising administering a therapeutically effective amount of a composition, a pharmaceutical composition, a nucleic acid, a vector, or a cell as described herein above to a subject in need thereof, wherein interaction between type I Fc receptor or type II Fc receptor and FcRn with an immunocomplexed antibody is reduced or inhibited. In one embodiment, the type I Fc receptor is selected from the group consisting of CD32, CD32a, CD32a^{H}, CD32a^{R}, CD16, CD16a, CD16a^{V158}, CD16a^{F158}, and CD16b. In another embodiment, the type II Fc receptor comprises DC-SIGN. In another embodiment, the subject has or has been diagnosed with an autoimmune disease, an IgG mediated autoimmune disease and or an inflammatory condition. In another embodiment, the IgG-mediated autoimmune disease or inflammatory condition is selected from Kawasaki disease, Sjogren's disease, Guillain-Barre, inflammatory bowel disease (IBD), Crohn's disease, ulcerative colitis, systemic lupus erythematosus (SLE), lupus arthritis, lupus nephritis, idiopathic thrombocytopenic purpura, rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behget's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, and neonatal myasthenia gravis.

In another aspect, described herein is a method to inhibit or reduce CD32b and FcRn interactions with immunocomplexed IgG, the method comprising administering a therapeutically effective amount of a composition, a pharmaceutical composition, a nucleic acid, a vector, or a cell as described herein above to a subject in need thereof, wherein the bispecific antibody construct is specific for CD32b and FcRn. In one embodiment, the subject has or has been diagnosed with cancer. In another embodiment, the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer. In one embodiment, the administration blocks tolerance and permits anti-tumor immunity.

IN another aspect, described herein is a method of treating cancer comprising administering a therapeutically effective amount of a composition, a pharmaceutical composition, a nucleic acid, a vector, or a cell as described herein above to a subject in need thereof, wherein the bispecific antibody construct is specific for CD32b and FcRn. In one embodiment, the subject has or has been diagnosed with cancer. In another embodiment, the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer. In one embodiment, the administration blocks tolerance and permits anti-tumor immunity.

In another aspect, described herein is a method to inhibit or reduce CD23 and FcRn interactions with an immunocomplexed IgE, comprising administering a therapeutically effective amount of a composition, a pharmaceutical composition, a nucleic acid, a vector, or a cell as described herein above to a subject in need thereof, wherein the bispecific antibody construct is specific for CD23 and FcRn. In one embodiment, the subject has or has been diagnosed with an IgE-mediated allergy. In another embodiment, the subject has or has been diagnosed with atopic dermatitis, a food allergy, an insect sting allergy, a skin allergy, a pet allergy, a dust allergy, an eye allergy, a drug allergy, allergic rhinitis, a latex allergy, a mold allergy, a sinus infection, or a cockroach allergy.

In another aspect, described herein is a method of treating an allergy, comprising administering a therapeutically effective amount of a composition, a pharmaceutical composition, a nucleic acid, a vector, or a cell as described herein above to a subject in need thereof, wherein the bispecific antibody construct is specific for CD23 and FcRn. In another embodiment, the subject has or has been diagnosed with an IgE-mediated allergy. In another embodiment, the subject has or has been diagnosed with atopic dermatitis, a food allergy, an insect sting allergy, a skin allergy, a pet allergy, a dust allergy, an eye allergy, a drug allergy, allergic rhinitis, a latex allergy, a mold allergy, a sinus infection, or a cockroach allergy.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “specificity” refers to the number of different types of antigens or antigenic determinants to which an antibody or antibody fragment thereof as described herein can bind. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof: the lesser the value of the K_{D}, the stronger the binding strength between an antigenic determinant and the antigen-binding molecule. Alternatively, the affinity can also be expressed as the affinity constant (K_{A}), which is 1/K_{D}). Accordingly, an antibody or antibody fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10000 times or more better than the affinity with which said amino acid sequence or polypeptide binds to another target or polypeptide.

Antibody affinities can be determined, for example, by a surface plasmon resonance based assay (such as the BIACORE assay described in PCT Application Publication No. WO2005/012359); enzyme-linked immunosorbent assay (ELISA); and competition assays (e.g., RIA's), for example.

As used herein, “avidity” is a measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D}) of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (K_{A}) of 10⁻⁵ to 10¹² liter/moles or more, such as 10⁷ to 10¹² liter/moles or 108 to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (RIA), enzyme immunoassays (EIA) and sandwich competition assays, and the different variants thereof known in the art; as well as other techniques as mentioned herein.

Accordingly, as used herein, “selectively binds” or “specifically binds” refers to the ability of an anti-body polypeptide (e.g., a recombinant antibody or portion thereof) described herein to bind to a target, such as a receptor molecule present on the cell-surface, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, the term “selectively inhibits” means that an agent, such as a bispecific antibody agent, inhibits, as that term is used herein, the association of a first ligand-receptor pair but does not substantially inhibit the association of a relevant second ligand-receptor pair. In the context of a preferred bispecific anti-FcRn, anti-Type I, or anti-Type II Fc receptor construct, the bispecific construct inhibits the binding of immunocomplexed IgG but does not substantially inhibit binding of monomeric IgG to FcRn, thereby selectively inhibiting the binding of immunocomplexed IgG to FcRn. In this context, the term “does not substantially inhibit” or “does not substantially modulate” means that the bispecific, at a concentration that reduces immunocomplexed IgG to FcRn binding by at least 80%, causes no more than a 20% reduction in monomeric IgG binding to FcRn, and preferably no more than 10% inhibition of monomeric IgG finding to FcRn, more preferably no more than 5%, 4%, 3%, 2%, 1% or less inhibition of monomeric IgG binding to FcRn as compared to such binding in the absence of the bispecific antibody agent.

As used herein, the term “does not result in hypogammaglobulinemia” means that a given treatment does not reduce gammaglobulins generally to a level recognized by clinicians as immunocompromised.

As used herein, the term “preferentially binds” means that in the context of a given bispecific construct, first and second target- or antigen-binding domains of one construct molecule bind to FcRn and a Type I or Type II Fc receptor that are in a tripartite or ternary complex with an IgG molecule, as opposed to FcRn and Type I or Type II Fc receptor molecules that are not bridged by or complexed with one IgG molecule. Given the kinetics of binding by two different domains, the preference for binding targets in close proximity, e.g., in a single ternary complex, as opposed to targets that are further apart is determined by the separation of the first and second binding domains in the bispecific construct, with shorter distances (determined, e.g., by shorter linker structures) favoring association with closely apposed target domains. That is, while two binding domains separated by a long linker can physically associate with two closely apposed binding targets, it will not do so preferentially as compared to a construct with the same two binding domains separated by a shorter linker (provided that the linker is long enough to bridge the distance between the closely apposed targets).

As used herein, the terms “immunocomplexed antibody” or “immune complex antibody” refers to an antibody bound via its antigen-binding domain(s) to an antigen molecule. “Immunocomplexed IgG” or “immune complex IgG” refer more specifically to the complex of an IgG antibody molecule with an antigen molecule; other variants, such as immune complex IgE, are referred to accordingly. The terms “immunocomplexed antibody” and “immune complex antibody” are in contrast to the terms “monomeric antibody” or “monomeric immunoglobulin,” which refer to antibodies that are not bound to antigen.

As used herein, the term “linker” refers to a chemical or peptide structure that covalently joins two polypeptide moieties. For example, a V_{H} domain and a V_{L} domain of an antibody can be joined by a peptide linker to form a V_{H}/V_{L} single chain antigen binding domain (e.g., as an scFv). Lengths of linkers can be varied to modify the ability of linked domains to form, e.g., intramolecular or intermolecular dimers. For example, a diabody includes a short linker peptide between V_{H} and V_{L} domains, usually 5 amino acids, that will not permit the V_{H} and V_{L} domains to pair to form an antigen-binding domain; expression of two different V_{H}-V_{L} constructs with this short linker arrangement in a cell permits the V_{H} domain of a first V_{H}-V_{L} polypeptide chain to dimerize with the V_{L} domain of the second V_{H}-V_{L} polypeptide chain, and the corresponding V_{L} domain of the first V_{H}-V_{L} polypeptide chain to dimerize with the V_{H} domain of the second V_{H}-V_{L} polypeptide chain, thereby generating a bispecific construct. In contrast, when the V_{H} and V_{L} domains are separated by a longer peptide linker, most often 15-20 amino acids, the V_{H} domain and the V_{L} domain on the same polypeptide chain can dimerize to form an scFv.

As used herein, the term “modulate the interaction” means that the interaction between two moieties, such as an immunoglobulin molecule and a receptor, such as FcRn or an Fcγ receptor, is inhibited or promoted, as the case may be, wherein inhibiting or promoting mean a change of at least 10% in the presence of a modulating agent, and preferably at least 20%, 30%, 40%, 50%, 60%, 70%, 80%, 90% or more, relative to the absence of the modulating agent. Such modulation can be measured using standard assays of binding kinetics.

In some embodiments, the antigen specific domains of a bispecific antigen-binding construct comprise one or more non-immunoglobulin antigen binding scaffolds. In some embodiments of these engineered constructs, the CDRs of an antibody are arranged on a non-immunoglobulin scaffold molecule, such as a scaffold polymer or polypeptide. In others, a non-immunoglobulin scaffold protein structure includes regions that are randomized and expressed, e.g., in a phage display system to select for members that bind a given target with high affinity. Non-limiting examples of non-immunoglobulin antigen-binding scaffolds include a DARPIN, an affibody, an affilin, an adnectin, an affitin, an Obody or Obodies, a repebody, a fynomer, an alphabody, an avimer, an atrimer, a centyrin, a pronectin, an anticalin, a kunitz domain, or an Armadillo repeat protein. Examples of non-immunoglobulin antigen binding scaffolds are described in WO 2017/172981 and the tables therein, which are incorporated herein by reference

The term “anti-FcRn therapy” refers to administration of an agent that, at a minimum, blocks the interaction of FcRn with immunoglobulin, such as IgG, and thereby interferes with the FcRn-mediated direction of internalized immunoglobulin away from endosomal degradation. In some embodiments, anti-FcRn therapy can inhibit other FcRn-mediated processes, including, but not limited to interaction of FcRn with other biomolecules, such as alphafetoprotein (AFP).

As used herein, a “blocking” antibody or an antibody “antagonist” is one which inhibits or reduces the biological activity of the antigen(s) to which it binds. For example, a bispecific anti-FcRn, anti-CD32a blocking or antagonist antibody binds FcRn and CD32a and inhibits recycling of immune complex IgG. In certain embodiments, the blocking antibodies or portions thereof as described herein completely inhibit the interaction between an immunoglobulin, such as IgG, FcRn and a given Type I or Type II Fc receptor. In certain embodiments, the blocking antibodies or portions thereof as described herein reduce or decrease the interaction between an immunoglobulin, such as IgG, FcRn and a given Type I or Type II Fc receptor.

Assays to detect or measure binding of an agent, such as an antibody construct to FcRn and/or a Type I or Type II Fc receptor are known in the art. Non-limiting examples include co-immunoprecipitation and affinity biosensor methods. Affinity biosensor methods can be based on the piezoelectric effect, electrochemistry, or optical methods, such as ellipsometry, optical wave guidance, and surface plasmon resonance (SPR).

As used herein, the term “bispecific polypeptide agent” refers to a polypeptide that comprises a first polypeptide domain which has a binding site that has binding specificity for a first target, and s second polypeptide domain which has a binding site that has specificity for a second target, i.e., the agent has specificity or is specific for two targets. The first target and second target are not the same, but are both present in an in vivo situation, such that one bispecific agent can encounter and simultaneously bind both targets. Due to the avidity effect of having two closely apposed binding domains, a bispecific agent, including a bispecific polypeptide agent, will bind to targets, including antigen epitopes that are, themselves, closely apposed, more strongly (i.e., with greater avidity) than the bispecific agent will bind either target or antigen when the targets or antigens are not in close apposition to the other. Such difference in avidity thereby provides a preference or selectivity of the bispecific agent, such as a bispecific polypeptide agent, that can be exploited for therapy.

As used herein, the term “multispecific polypeptide agent” refers to a polypeptide that comprises at least a first polypeptide domain having a binding site that has binding specificity for a first target, and a second polypeptide domain having a binding site that has binding specificity for a second target. A multispecific polypeptide agent can include further, e.g., third, fourth, etc. binding sites for additional targets. The various targets are not the same (i.e., are different targets (e.g., proteins)), but are each present in an in vivo situation, such that one bispecific agent can potentially encounter and potentially bind simultaneously to each of the targets. In one embodiment, the third, fourth or further binding site comprises a site that targets the multispecific agent to a desired location, e.g., via binding specificity for a cell- or tissue-specific marker. A non-limiting example of a multispecific polypeptide agent is a multispecific antibody construct. For the avoidance of doubt, a bispecific polypeptide agent is a type of multispecific polypeptide agent.

As used herein, the term “target” refers to a biological molecule (e.g., peptide, polypeptide, protein, lipid, carbohydrate, etc.) to which a polypeptide domain which has a binding site can selectively bind. The target can be, for example, an intracellular target (e.g., an intracellular protein target) or a cell surface target (e.g., a membrane protein, a receptor protein).

The term “universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. 196:910-917 (1987). The Kabat database is now also maintained on the world wide web. The compositions and methods described herein provide for the use of a single framework, or a set of such frameworks, which have been found to permit the derivation of virtually any binding specificity though variation in the hypervariable regions alone. The universal framework can be a V_{L} framework (V_{λ} or V_{κ}), such as a framework that comprises the framework amino acid sequences encoded by the human germline DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline J_{κ}1, J_{κ}2, J_{κ}3, J_{κ}4, or J_{κ}5 immunoglobulin gene segments. In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segments. In some embodiments, the V_{H} framework can further comprise the framework amino acid sequence encoded by the human germline J_{H}1, J_{H}2, J_{H}3, J_{H}4, J_{H}4b, J_{H}5 or J_{H}6 immunoglobulin gene segments.

An “Fv” fragment is an antibody fragment which contains a complete antigen recognition and binding site. This region consists of a dimer of one heavy and one light chain variable domain in tight association, which can be covalent in nature, for example in a single-chain Fv or scFv (see below). It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. For the methods and compositions described herein, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDRI, CDR2, and CDR3) refers to the amino acid residues of an antibody variable domain the presence of which are necessary for antigen binding. Each variable domain typically has three CDR regions identified as CDRI, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop as defined by Chothia and Lesk.

“Framework regions” (hereinafter FR) are those variable domain residues other than the CDR residues. Each variable domain typically has four FRs identified as FRI, FR2, FR3 and FR4. If the CDRs are defined according to Kabat, the light chain FR residues are positioned at about residues 1-23 (LCFR I), 35-49 (LCFR2), 57-88 (LCFR3), and 98-107 (LCFR4) and the heavy chain FR residues are positioned about at residues 1-30 (HCFR I), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102113 (HCFR4) in the heavy chain. In some instances, when the CDR comprises amino acids from both a CDR as defined by Kabat and those of a hypervariable loop, the FR residues will be adjusted accordingly. For example, when CDRHI includes amino acids H26-H35, the heavy chain FRI residues are at positions 1-25 and the FR2 residues are at positions 36-49.

A “Fab” of “Fab fragment” fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which permits the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (VH—CHI-VH-CH1) which, together with complementary light chain polypeptides, form a pair of antigen binding regions. Linear antibodies can be bispecific or monospecific.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein in relation to antibody domains, “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

The process of designing, selecting and/or preparing a bispecific of multispecific polypeptide agent as described herein is also referred to herein as “formatting” the amino acid sequence, and an amino acid sequence that is made part of a bispecific or multispecific polypeptide agent described herein is said to be “formatted” or to be in the format of that bispecific or multispecific polypeptide agent. Examples of ways in which an amino acid sequence can be formatted and examples of such formats will be clear to the skilled person based on the disclosure herein; and such formatted amino acid sequences form a further aspect of the bispecific or multispecific polypeptide agents described herein.

In some embodiments of the aspects described herein, a polypeptide agent can be formatted as a bispecific polypeptide agent as described herein, and in US 2010/0081796 and US 2010/0021473, the contents of which are herein incorporated in their entireties by reference. In other embodiments of the aspects described herein, a polypeptide agent can be formatted as a multispecific polypeptide agent, for example as described in WO 03/002609, the entire teachings of which are incorporated herein by reference.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of autoimmune disease, cancer, or allergy. A subject can be male or female.

A subject can be one who has been previously diagnosed with or identified as suffering from or having a condition in need of treatment (e.g. autoimmune disease, cancer, or allergy) or one or more complications related to such a condition, and optionally, have already undergone treatment for autoimmune disease, cancer, or allergy or the one or more complications related to autoimmune disease, cancer, or allergy. Alternatively, a subject can also be one who has not been previously diagnosed as having autoimmune disease, cancer, or allergy or one or more complications related thereto. For example, a subject can be one who exhibits one or more risk such diseases or disorders.

A “subject in need” of treatment for a particular condition can be a subject having that condition, diagnosed as having that condition, or at risk of developing that condition.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having a disease or disorder as described herein, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding a polypeptide as described herein (e.g. a bispecific antibody polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. autoimmune disease, cancer, or allergy. The term “treating” includes reducing or alleviating at least one adverse effect or symptom of a condition, disease or disorder associated with an autoimmune disease, cancer, or allergy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-FIG. 1C is a series of schematics showing a model of the proposed CD32a-IgG-FcRn ternary complex. FIG. 1A shows the superposition of the FcRn-hIgG1 Fc crystal structure (PDB ID 4N0U) and hIgG1 Fc of CD32aR complex (PDB ID 3RY6), which were done to generate on FcRn-IgG Fc-CD32aR structural model. FIG. 1B shows the superposition of intact human IgG1 antibody (PDB ID 1HZH) on FcRn-IgG Fc-CD32aR structural model, which reveals enough space is available to adjust Fab arms of hIgG1 and accommodate ternary complex formation. FIG. 1C shows the crystal structure of CD32aR (PDB ID 3RY6) complexed with Fc of hIgG1. The upper inset details the residue R131 of CD32aR in proximity to residue D265 of Fc, and the lower inset details the same location for the structure model of CD32aH variant at acidic pH, showing proximity of residues H131 and S267 based on a model of CD16B, which is homologous to CD32a at position 131 occupied by residue H. FIG. 1D shows the CD16B-hIgG1 Fc crystal structure (PDB ID 1T83) superimposed onto the hIgG1 Fc-CD32aR and FcRn complex structural model. The inset shows the proximity of residues H131 of CD16B and S267 of hIgG1 Fc as observed in crystal structure.

FIG. 2 shows an image of a multiple sequence alignment of mouse and human IgG amino acid sequences. An asterisk (*) denotes a homologous residue. Imputed contact residues are indicated at residue numbers 265, 267, and 270. Unique residues are indicated by their single letter abbreviation.

FIG. 3A-FIG. 3B is a series of images of interface analyses. FIG. 3A shows the interface analysis of hIgG1 Fc and CD16B complex (PDB ID 1T83) with PDB PISA. FIG. 3B shows the interface analysis of hIgG1 Fc and CD32aR complex (PDB ID 3RY6) with PDB PISA.

FIG. 4A-FIG. 4C is a series of schematics showing design of bispecific antibodies. FIG. 4A shows the distance between FcRn binding site residues for IgG Fc and the CD32a binding site for IgG Fc. FIG. 4B shows targeting of the FcRn binding site for IgG Fc and CD32a or CD16 binding site for IgG Fc by a bispecific antibody. FIG. 4C shows FcRn and CD32a, CD16a and CD16b interface residues.

FIG. 5 is a schematic showing the design of FcRn-CD32 DVD-Ig bispecific antibodies.

FIG. 6A-FIG. 6F is a series of line graphs and images showing that CD32a and FcRn form a ternary complex with IgG under acidic conditions. FIG. 6A shows binding at pH 5.5 of serially diluted hIgG1^{WT} IC (controls: hIgG1^{IHH} and hIgG1^{N297A} as monomers and IC, hFcRn only) to C-terminus biotinylated CD32a^{H}, which had been captured on neutravidin-coated ELISA plates, and detected by a single concentration of a hFcRn reporter complex (recombinant hFcRn pre-incubated at pH 5.5 with an alkaline phosphatase (ALP)-conjugated hFcRn-specific nanobody). FIG. 6B shows binding at pH 5.5 of serially diluted hIgG1^{WT} IC (controls: hIgG^{1HH} and hIgG1^{N297A} as monomers and IC, hFcRn only) to C-terminus biotinylated CD32a^{R}, which had been captured on neutravidin-coated ELISA plates, and detected by a single concentration of a hFcRn reporter complex (recombinant hFcRn pre-incubated at pH 5.5 with an alkaline phosphatase (ALP)-conjugated hFcRn-specific nanobody). FIG. 6C shows binding responses at pH 5.5 of serially diluted anti-NIP mIgG1, mIgG2a or mIgG2b IC (controls: monomeric mIgG1, mIgG2a or mIgG2b, hFcRn only) to C-terminus biotinylated CD32a^{H}, captured on neutravidin-coated ELISA plates and detected by a single concentration of the hFcRn-ALP-nanobody complex as in FIG. 6A-FIG. 6B. FIG. 6D shows binding responses at pH 5.5 of serially diluted anti-NIP mIgG1, mIgG2a or mIgG2b IC (controls: monomeric mIgG1, mIgG2a or mIgG2b, hFcRn only) to C-terminus biotinylated CD32a^{R}, captured on neutravidin-coated ELISA plates and detected by a single concentration of the hFcRn-ALP-nanobody complex as in FIG. 6A-FIG. 6B. FIG. 6E shows RAW264.7 cells transfected with either CD32aH (H) or CD32a^{R} (R), treated for 30 minutes with protein-A-conjugated Dynabeads coated with hIgG1 IC (formed with hIgG1 and fluorescently-labeled F(ab′)2 from goat against human F(ab′)2) imaged by confocal microscopy. Scale bar 3 micrometer (μm). FIG. 6F shows confocal microscopic images of proximity ligation assay in cells treated for 15 minutes with soluble IC formed as in FIG. 6E. Scale bar=3 μm. Images are representative of two independent experiments. Vector=control vector, R=CD32a^{R}, H=CD32a^{H}.

FIG. 7A-FIG. 7D is a series of bar graphs showing that FcRn and CD32a cooperate in APC responses to IC. FIG. 7A shows IFNγ production by CD8+OT-I T cells after 48 hours of co-culture with primary CD11c⁺ APC expressing CD32a^{H}, pre-treated with anti-FcRn mAb DVN24 or isotype control antibody 30 minutes prior to antigen loading with OVA, hIgG^{1HH} IC (IHH), hIgG1^{WT} IC (WT), at pH 7.4. FIG. 7B shows IFNγ production by CD8+OT-I T cells after 48 hours of co-culture with primary CD11c⁺ APC expressing CD32a^{H}, pre-treated with anti-FcRn mAb DVN24 or isotype control antibody 30 minutes prior to antigen loading with OVA, hIgG1^{IHH} IC (IHH), or hIgG1^{MST/HN} IC (MST/HN), at pH 7.4 FIG. 7C shows IFNγ production by CD8⁺ OT-I T cells after 48 hours of co-culture with primary FcγR^{KO} APC (Fcgrt^{+/+}/Fcgr1^{-/-}/Fcgr2b^{-/-}/Fcgr3^{-/-}/Fcer1g^{-/-}), pre-treated with DVN24 or isotype control 30 minutes prior to antigen loading with OVA, hIgG1^{WT} IC, hIgG1^{IHH} IC, or hIgG1^{MST/HN} IC, at pH 7.4. FIG. 7D shows IFNγ production by CD8⁺ OT-I cells co-cultured for 48 hours with FcγR^{KO} APC that had been loaded with hIgG1^{WT} or hIgG1^{IHH} IC at pH 5.5 or 7.4. R=CD32a^{R}, H=CD32a^{H}. Arithmetic mean±standard error of the mean (SEM) are shown. All experiments were repeated twice and analyzed by 2-way ANOVA followed by Holm-Sidak post-hoc analysis. P<*0.05,**0.01, ***0.001, ‡0.0001.

FIG. 8A-FIG. 8J is a series of graphs and images showing that CD32a^{H} is more pro-inflammatory and shows greater dependence on FcRn than CD32a^{R}. FIG. 8A shows IL-2 production by CD4⁺ DO11.10 T cells after 24 hours of co-culture with RAW264.7 cells expressing CD32a^{R} (R) or CD32a^{H} (H), after the cells had been pre-treated with anti-NIP hIgG1^{WT}, hIgG1^{IHH}, or hIgG1^{N297A} IC (100 μg/ml IgG, 10 μg/ml NIP-OVA). FIG. 8B shows IL-2 production by OVA-peptide-restricted CD4⁺ T cells after 24 hours of co-culture with CD32a variant-expressing RAW264.7 cells pretreated with 100 μg/ml hIgG1^{WT} complexed with the indicated concentration of NIP-OVA. FIG. 8C shows the percent change in cell surface binding at pH 5.5 versus 7.4 of fluorescent IC formed with hIgG1 and hIgG2 to CD32a^{H}- or CD32a^{R}-expressing MDCK-II cells at 4° C. FIG. 8D shows hFcRn-binding responses at pH 5.5 to fixed concentration of CD32a-hIgG1^{WT} complexes immobilized on neutravidin-coated ELISA plates. § indicates that non-linear regression analysis-generated (4-parameter) best-fit curves were significantly different (extra sum-of-squares F test; P=0.001). FIG. 8E shows the percent inhibition of IFNγ production by CD8+OT-I T cells co-cultured with DVN24- or isotype control-pretreated CD11c⁺ APC 30 minutes prior to hIgG1^{WT} IC APC loading, calculated as [(isotype-treated IFNγ minus DVN24-treated IFNγ)×100]÷(isotype-treated IFNγ). FIG. 8F shows p-Syk immunoblot (IB) in CD32a variant-expressing HEK293T cells 10 minutes after stimulation with mIgG1 IC or the indicated controls (see e.g., FIG. 11C, FIG. 11D). FIG. 8G shows IFNγ production by CD8+OT-I T cells after 48 hours of co-culture with HEK293TH2-Kb cells expressing CD32aH (H), CD32aR (R) or no FcγR (Vector) and loaded at pH 7.4 with mIgG1 IC at the indicated concentrations. FIG. 8H shows IFNγ production by CD8+OT-I T cells after 48 hours of co-culture with primary CD11c+ CD32aTg APC that were loaded at pH 7.4 with mIgG1 IC at the indicated concentrations. FIG. 8I shows Percent change in cell surface binding at pH 5.5 versus 7.4 of fluorescent IC formed with mIgG1 to CD32aH- or CD32aR-expressing MDCK-II cells at 4° C. FIG. 8J shows IFNγ production by CD8+OT-I T cells after co-culture with CD11c+CD32aTg APC loaded with mIgG1 IC at pH 7.4 or pH 5.5. Vector=control vector, R=CD32a^{R}, H=CD32a^{H}. Arithmetic mean±SEM. 2-way ANOVA (FIG. 8A, FIG. 8B, FIG. 8E, FIG. 8G, FIG. 8H, FIG. 8J) or multiple t-tests (FIG. 8D) with correction for multiple comparisons by the two-stage linear step-up procedure of Benjamin, Krieger and Yekutieli with false discovery rate (FDR)<0.05 or (FIG. 8C, FIG. 8) 2-way ANOVA with Fisher LSD test. P<*0.05,**0.01,***0.001, ‡0.0001.

FIG. 9A-FIG. 9L is a series of graphs and images showing that FcRn blockade ameliorates IC-mediated colitis and RA in a CD32a allele-specific manner. FIG. 9A-FIG. 9E shows DVN24 vs. isotype treatment in anti-flagellin IgG/DSS-induced colitis. FIG. 9A shows total anti-flagellin IgG levels in serum before (day −1) and after (day 9) DSS administration in BM chimeric mice (CD32a^{R-Tg}, n=8; CD32a^{H-Tg}, n=7), as per DVN24/isotype treatment group assignments. FIG. 9B shows weight loss during colitis (two independent experiments). FIG. 9C shows representative H&E staining of colonic tissue. FIG. 9D shows blinded histological score of colonic tissue (at least three consecutive sections). FIG. 9E shows percent inhibition of inflammatory cytokine transcript levels in CD11c⁺ APC isolated from mesenteric lymph nodes (MLN); a higher value indicates greater inhibition. mRNA levels were measured by qPCR in triplicate and normalized to intrinsic GAPDH expression, and then to isotype-treated mice to determine the degree of inhibition. FIG. 9F-FIG. 9J shows DVN24 vs. isotype treatment in K/BxN arthritis. In BM chimeric CD32a^{Tg} mice (n=4/group), K/BxN serum transfer arthritis endpoints were performed in two independent experiments. FIG. 9F shows ankle diameter. FIG. 9G shows Area Under the Inflammation*Time Curve (AUC). FIG. 9H shows ankle joint histopathology (day 6). FIG. 9I shows blinded scoring of inflammation and bone erosion (at least three consecutive sections). FIG. 9J shows mobility (increased number (#) of side touches reflects less disease). FIG. 9K-FIG. 9L shows Fcgrt−/− vs. wild type in K/BxN arthritis. The K/BxN arthritis model is in BM chimeric recipients of BM from CD32a^{H-Tg}/Fcgrt^{-/-} or CD32a^{R-Tg}/Fcgrt^{-/-} BM donors (n=4/group). FIG. 9K shows ankle diameter. FIG. 9L shows inflammation score AUC. R=CD32a^{R-Tg}, H=CD32a^{H-Tg} Arithmetic mean±SEM. 2-way ANOVA with (FIG. 9A, FIG. 9B, FIG. 9D, FIG. 9G, FIG. 9, FIG. 9L) Holm-Sidak post-hoc analysis, or (FIG. 9E, FIG. 9F, FIG. 9K) multiple student t test with correction for multiple comparisons by the two-stage linear step-up procedure of Benjamin, Krieger and Yekutieli with FDR<0.05. P<*0.05, **0.01, ***0.001, ‡0.0001.

FIG. 10A-FIG. 10F is a series of graphs and images showing that CD32a-IgG-FcRn bridging occurs at acidic pH. FIG. 10A shows ELISA experimental schematic design. Recombinant C-terminus-biotinylated CD32a variants were captured on neutravidin-coated plates. Analyte(s) were then injected as indicated and specified in the material and methods. FIG. 10B shows Surface Plasma Resonance (SPR) experimental schematic design. Recombinant C-terminus-biotinylated CD32a variants were captured on neutravidin amine-coupled CM5 sensor chips. Analyte(s) were then injected as indicated and specified in the material and methods. FIG. 10C shows SPR sensorgrams of mFcRn binding at pH 5.5 to immobilized CD32a^{H} with or without monomeric anti-NIP mIgG1, mIgG2a and mIgG2b. FIG. 10D shows SPR sensorgrams of mFcRn binding at pH 5.5 to immobilized CD32a^{R} with or without monomeric anti-NIP mIgG1, mIgG2a and mIgG2b. FIG. 10E shows superposition of the crystal structures of FcRn-hIgG1 Fe (PDB ID: 4N0U) and CD32aR-hIgG1 Fe (PDB ID: 3RY6). β2-microglobulin is removed for clarity. FIG. 10F shows confocal microscopic images of proximity ligation assay in cells treated for 15 minutes as in FIG. 6F, except without IC. Scale bar=3 μm. Images are representative of two independent experiments. Vector=control vector, R=CD32a^{R}, H=CD32a^{H}.

FIG. 11A-FIG. 11S is a series of graphs and immunoblots showing that CD32a^{H} induces greater immune activation due to increased bridging at acidic pH. FIG. 11A shows representative histograms of CD32a and H2-Kb expression in stably transfected HEK293T^{H2-Kb}/R or HEK293^{TH2-Kb/H} cells. FIG. 11B shows cumulative mean fluorescence intensity (MFI) of CD32a and H2-Kb expression in stably transfected HEK293T^{H2-Kb/R} or HEK293^{TH2-Kb/H} cells. FIG. 11C shows an immunoblot (IB) of phosphorylated Syk (p-Syk) in immunoprecipitated (IP) Syk from the lysate of HEK293T cells expressing CD32a^{R} or CD32a^{H} that had been stimulated with hIgG1^{WT}, hIgG1^{IHH}, or hIgG1^{N297A} IC for 10 minutes. FIG. 11D shows an immunoblot (IB) from a second independent p-Syk co-IP experiment. FIG. 11E shows densitometric analysis of the p-Syk from FIG. 11C and FIG. 11D. FIG. 11F shows representative histograms of transfected CD32a alleles in RAW264.7 cells. FIG. 11G shows cumulative MFI of transfected CD32a alleles in RAW264.7 cells. FIG. 11H shows representative histograms of CD32a expression in stably transfected MDCK-II cells. FIG. 11I shows cumulative MFI of CD32a expression in stably transfected MDCK-II cells. FIG. 11J shows relative MFI of hIgG1, hIgG2 binding to CD32a variant-transfected MDCK-II at pH 7.4 and 5.5, at 4° C., normalized to IC binding to vector-transfected controls. FIG. 11K shows CD32a expression as in FIG. 11H and FIG. 11. FIG. 11L shows relative MFI of fluorescently labeled goat F(ab′)2 (used in IC formation) against hF(ab′)2 without IgG, incubated with MDCK-II cells expressing CD32a variants (or vector control-transfected MDCK-II cells). FIG. 11M shows representative histograms of primary CD32a^{Tg} CD11c⁺ APC. FIG. 11N shows MFI of primary CD32a^{Tg} CD11c⁺ APC. FIG. 11O shows IFNγ production by CD8+OT-I T cells after 48 hours of co-culture with primary CD11c⁺ APC expressing CD32a^{R}, loaded with hIgG1^{WT}, hIgG^{1HH} or hIgG1^{MST/HN} IC variants in presence or absence of FcRn blockade (DVN24) or isotype control beginning 30 minutes before IC loading (see e.g., FIG. 7B for data from a parallel experiment with identically-treated primary CD11c⁺ APC expressing CD32a^{H}). FIG. 11P shows relative MFI of fluorescent mIgG1 IC binding to CD32a variant-transfected MDCK-II at pH 7.4 at 4° C., normalized to binding to vector-transfected controls. FIG. 11Q shows CD32a expression of FIG. 11P. FIG. 11R shows relative MFI of fluorescently labeled goat F(ab′)2 (used in IC formation) against mF(ab′)2 without IgG, added to MDCK-II cells expressing CD32a variants (or vector control-transfected MDCK-II cells). FIG. 11S shows relative MFI of fluorescent mIgG1 IC binding to CD32a variant-transfected MDCK-II at pH 7.4 and 5.5, at 4° C. Flow cytometry experiments were performed in triplicate. Vector=control vector, R=CD32aR, H=CD32a^{H}. Geometric mean (FIG. 11B, FIG. 11E, FIG. 11G, FIG. 11I-FIG. 11L, FIG. 11O-FIG. 11S) ±standard error of the mean (SEM). 1-way ANOVA with (FIG. 11I, FIG. 11N) Holm-Sidak or (e) the two-stage linear step-up procedure of Benjamin, Krieger and Yekutieli with false discovery rate <0.05. 2-way ANOVA with (FIG. 11B, FIG. 11K, FIG. 11L, FIG. 11O, FIG. 11Q, FIG. 11R) Holm-Sidak or (FIG. 11J, FIG. 11P, FIG. 11S) Fisher LSD post-hoc analysis. P<*0.05, **0.01. P<*0.05, **0.01, ***0.001, ‡0.0001.

FIG. 12A-FIG. 12K is a series of images and graphs showing that CD32a^{H} exhibits increased dependence on FcRn and increased sensitivity to FcRn blockade in vivo. FIG. 12A shows a schematic representation of flagellin-immunized/DSS-induced colitis model in bone marrow (BM) chimeric mice. BM from CD45.2+CD32a^{R-Tg} or CD32a^{H-Tg} mice was adoptively transferred to wild type (WT) CD45.1⁺ recipient mice, and CD45.2⁺ BM engraftment before immunization and FcRn blockade (DVN24/Isotype; x=excluded animal). Mice were immunized with S. typhimurium flagellin 28 and 14 days before 4% DSS exposure. One day before exposure to DSS mice began to receive daily i.p. injections of either 0.2 mg/mouse/day DVN24 or isotype control antibody which continued for the next seven days of DSS exposure. Sacrifice and tissue collection (n=4/group) occurred on day 9 (n=4/group), two days after DSS was exchanged for normal water. After day 9, percent weight change only was monitored until day 11(CD32a^{R-Tg}; CD32a^{H-Tg} n=3). FIG. 12B shows flow cytometry (cyt) verification of CD45.2+BM engraftment. FIG. 12C shows ELISA measurements of anti-flagellin mIgG levels in serum by subclass. FIG. 12D shows total serum IgG levels one day prior to DSS initiation. FIG. 12E shows quantification of IL-6 and MCP-1 cytokines in whole colonic tissue and colonic tissue explant culture, measured by cytokine bead array in triplicate on day 9. FIG. 12F shows a schematic representation of the K/BxN murine RA model, with DVN24 vs. isotype antibody treatment. CD32a^{R-Tg} or CD32a^{H-Tg} BM transfer to C57BL/6 WT mice occurred 6 weeks prior to DVN24/Isotype (n=4) initiation, which occurred daily beginning the day prior to K/BxN serum transfer (Tr; ⋅) and continued for 5 days, with sacrifice and tissue collection on day 6. FIG. 12G shows clinical inflammation score in DVN24 or isotype control antibody-treated CD32a^{R-Tg} or CD32a^{H-Tg} BM chimeric mice after K/BxN serum transfer. FIG. 12H shows microcomputed tomographic (μCT) images, as 2D and 3D reconstructions of the forepaw images, with arrows indicating erosions. FIG. 12I shows the sum of radiographic erosion scores for right and left forepaws for each mouse, averaged by group. FIG. 12J shows clinical inflammation scoring, and FIG. 12K shows mobility (# side touches) on day 6 (2 independent experiments) from K/BxN arthritis experiment in wild type C57BL/6 recipients of BM from CD32a^{H-Tg}/Fcgrt^{-/-} or CD32a^{R-Tg}/Fcgrt^{-/-} BM donors (n=4). Arithmetic mean±SEM. 2-way ANOVA with (FIG. 12B-FIG. 12E, FIG. 12, FIG. 12K) Holm-Sidak post-hoc analysis, or (FIG. 12G, FIG. 12J) multiple t-tests with correction for multiple comparisons by the two-stage linear step-up procedure of Benjamin, Krieger and Yekutieli with FDR <0.05. P<*0.05, **0.01, ***0.001, ****0.0001.

### DETAILED DESCRIPTION

The technology described herein relates, in part, to the discovery that Fcγ receptors and FcRn form a tripartite complex with immunocomplexed IgG. The discovery of this complex provides an approach for the selective manipulation of the immunoregulatory processes mediated by Fcγ receptors and by FcRn. In particular, an agent that selectively binds to both Fcγ receptor and FcRn can selectively target immunocomplexed versus monomeric IgG, e.g., for the treatment of IgG-mediated autoimmune disease, while avoiding hypogammaglobulinemia. Other therapeutic approaches permitted by the discovery that FcRn and Fcγ receptor interactions with antibody occur in close apposition in vivo include selectively targeting inhibitory Fcγ receptor CD32b for inhibition to promote anti-cancer immune activities. Further, where the domains of IgG that mediate the respective binding of FcRn and Fcγ receptors are shared by, for example, IgE, it is contemplated that allergy can also be treated by targeting an FcRn:IgE:FceR complex.

In various embodiments, therapeutic compositions and methods as described herein use bispecific binding agents, such as bispecific antibody constructs that recognize and specifically bind both FcRn and a given Fcγ receptor. The following describes considerations to permit one of ordinary skill in the art to make and use the compositions described for the treatment of autoimmune disease, cancer, chronic infection and/or allergy, among others.

### Fc Receptors

Receptors for the Fc region of antibodies (FcR) play a coordinating role in immunity. They are expressed on various types of cells and mediate functions ranging from endocytosis, phagocytosis, antibody-dependent cell-mediated cytotoxicity (ADCC), and cytokine production, to facilitation of antigen presentation. Antigen presentation refers to a process in which antigens are captured, targeted to appropriate compartments, and processed before binding to major histocompatibility complex (MHC) molecules for display by antigen-presenting cells to immunoreactive lymphocytes. FcR molecules can potently enhance antigen presentation. The type of FcR involved has been shown to be a crucial determinant for the types of epitopes presented by the antigen presenting cell (Amigorena, et al. (1998) J. Exp. Med. 187:505).

### Type I and Type H FcRs

Two structurally distinct sets of traditional FcRs have been recognized: Type I FcRs and Type II FcRs. Type I FcRs belong to the immunoglobulin receptor superfamily and are represented by the canonical Fc7 receptors, including the activating receptors CD64 (FcγTRI), CD32a (FcγRIIa), CD32c (FcγRIIc), CD16a (FcγRIIIa) and CD16b (FcγRIIIb), and the inhibitory receptor CD32b (FcγRIIb). Type II FcRs are represented by the family of C-type lectin receptors, which includes DC-SIGN (CD209) and CD23 (FcgR) (see e.g., Pincetic et al. (2014) Nature Immunology 15(8): 707-716).

CD32 (FcγRII) represents a low-affinity receptor, interacting mainly with immune-complexed IgG. It is the only receptor with an ITAM signaling motif in its ligand-binding chain. CD32a (FcγRIIa) represents the most widely distributed FcγR subclass and is present on most myeloid cells, i.e., neutrophils, eosinophils, monocytes and macrophages, as well as on platelets (see e.g., Deo, et al. (1997) Immunol. Today 18:127; King, et al. (1990) Cell Immunol. 128:462; Daeron, M. (1997) Annu Rev Immunol. 15:203). CD32b (FcγRIIb) bears an ITIM inhibitory motif within its intracellular tail and is expressed on B-cells and phagocytes (Tridandapani, et al. (2002) J. Biol. Chem. 277:5082V).

CD32a is functionally polymorphic: a single nucleotide polymorphism results in either an arginine (R) or histidine (H) residue at position 131 in the membrane-proximal Ig-like domain. Amino acid 131 is located in the IgG-docking site and greatly affects receptor affinity for IgG immune complexes (see e.g., Maxwell et al. (1999) Nat. Struct. Biol. 6:437-442; van der Pol et al. (2003) Immunogenetics 55:240-246).

CD32a-H131 (referred to hereafter as CD32^{H}) exhibits a higher affinity for human IgG2 and IgG3 than CD32a-R131 (referred to hereafter as CD32^{R}). Notably, CD32^{H}1 represents the sole leukocyte FcγR capable of binding IgG2. The functionally different CD32a alleles have been identified as risk factors for auto-immune and infectious diseases, as well as select polyneuropathies (see e.g., van der Pol and van de Winkel (1998) Immunogenetics 48:222-232; van Sorge et al. (2003) Tissue Antigens 61: 189-202). This polymorphism has also been linked to induction of side effects with therapeutic antibodies (Tax et al. (1997) Transplantation 63:106-112), and clinical efficacy of antibodies such as Rituxan® (Weng and Levy (2003) J Clin. Oncol. 21:3940-3947).

The CD32^{R} and CD32^{H} allotypes have initially been determined functionally, based on the differential interaction of CD32a allotypes with mouse IgG1 or human IgG2 antibodies in T-cell proliferation, EA-rosetting, or cellular cytotoxicity studies (see e.g., van de Winkel et al. (1987) Scand. J. Immunol. 26:663-672; Clark et al. (1989) J. Immunol. 143:1731-1734; Parren et al. (1991) Res. Immunol. 142:749-763; Warmerdam et al. (1991) J. Immunol. 147:1338-1343; Wurflein et al. (1998) Cancer Res. 58:3051-3058).

Both CD64 and CD32 have been implicated in auto-immune cytopenic diseases in mice and man (see e.g., Clynes, R. et al (1995) Immunity 3:21-26; Kumpel, B. M. et al. (1990) MoI. Immunol. 27:247-256). CD32a plays a role in clearance of immune-complexes, like human IgG-coated red blood cells in man (see e.g., Dijstelbloem, H. M. et al. (2000) Arthritis Rheum. 43:2793-2800). Lupus-associated immune complexes have been shown to activate human neutrophils in a CD32a-dependent manner (see e.g., Bonegio et al. (2019) The Journal of Immunology, 202: 675-683).

Upon exposure to antigens, specific IgG antibodies in the peripheral repertoire or generated early in the antibody response result in the formation of immune complexes; in turn, depending on their Fc conformations, these interact either with type I FcRs or type II FcRs on effector cells and on B cells to modulate both humoral immune processes and innate immune processes. Balanced positive and negative signaling through type I and type II FcRs is essential for the development of appropriate immune responses to soluble protein antigens or microorganisms.

### Neonatal Fc Receptor (FcRn)

The neonatal Fc receptor (FcRn) is an intracellular trafficking integral membrane receptor for IgG Fc. FcRn is a heterodimer of a membrane bound alpha-chain (GenBank accession no. NM004107) and soluble β2-microglobulin (β2m) (GenBank accession no. NM004048) and is structurally related to MHC class I molecules. FcRn regulates serum IgG concentrations by binding to and protecting endocytosed monomeric (i.e. non-antigen-bound) or immunocomplexed IgG from degradation in the lysosomal compartment, and transporting the IgG to the cell surface for release at neutral extracellular pH. Through this mechanism, FcRn is responsible for the long serum half-life of IgG. Accordingly, specific blockade of FcRn-IgG interaction can be used to promote degradation of pathogenic IgG antibodies. FcRn also binds multivalent IgG immune complexes (IC) within antigen presenting cells (APCs) such as dendritic cells (DCs), directing the bound IC into antigen processing pathways for presentation to T cells and activation of T cell mediated immune responses. Accordingly, specific blockade of FcRn-IC interaction can be used to inhibit T cell mediated immune responses, including reducing the production of inflammatory cytokines such as IL-6, IL-12, IFNγ, or TNFα.

FcRn was originally identified as a receptor functioning in neonatal life. It was first isolated from rodent gut as a heterodimer between a 12 kDa and a 40-50 kDa protein (Rodewald & Kraehenbuhl 1984, J. Cell. Biol. 99(1 Pt2): 159s-154s; Simister & Rees, 1985, Eur. J. Immunol. 15:733-738) and was cloned in 1989 (Simister & Mostov, 1989, Nature 337:184-187). Cloning and subsequent crystallization of FcRn revealed it to have an approximately 50 kDa major histocompatibility complex (MHC) class I-like heavy chain in non-covalent association with a 12 kDa β2-microglobulin light chain (Raghavan et al., 1993, Biochemistry 32:8654-8660; Huber et al., 1993, J. Mol. Biol. 230:1077-1083). Although first recognized in connection with fetal and neonatal life, FcRn is today known to continue to function throughout adult life. FcRn resides primarily in the early acidic endosomes where it binds to the Fc region of IgG in a pH-dependent manner, with micro- to nanomolar affinity at pH 6.5, while binding of FcRn to Fc at physiological pH is negligible. The bulk of FcRn is present in endosomes in most cells, and the interaction between FcRn and its IgG Fc ligands occurs within that acidic environment. In some cells, such as hematopoietic cells, significant levels of FcRn can be detected on the cell surface in addition to intracellular expression (Zhu et al., 2001, J. Immunol. 166:3266-3276). In this case, when the extracellular milieu is acidic, as in the case of neoplastic or infectious conditions, it is possible that FcRn can bind to IgG on the cell surface of these cell types.

During the first stages of life, FcRn confers passive immunity to offspring before and after birth by mediating transfer of IgG across the maternal placenta or neonatal intestinal walls. FcRn continues to function throughout adult life and is expressed in various tissues, e.g., the epithelium of the lung and liver, the vascular endothelium, as well as in monocytes, macrophages, and dendritic cells.

FcRn-deficient mice are more resistant to autoimmune diseases caused by pathogenic IgG autoantibodies because they are unable to maintain high concentrations of pathogenic serum IgG (see e.g., Christianson et al., 1996, J. Immunol. 156:4932-4939; Ghetie et al., 1996, Eur. J. Immunol. 26:690-696; Israel et al., 1996, Immunol. 89:573-578). Examples of autoimmune diseases caused by IgG antibodies include but as not limited to systemic lupus erythematosus (SLE), rheumatoid arthritis (RA), scleroderma, Sjogren's syndrome, and cryoglobulinemia. Administration of antibodies engineered to have modified Fc regions that bind with higher affinity to FcRn was found to ameliorate disease in a murine arthritis model (see e.g., Patel et al., 2011, J. Immunol. 187:1015-1022). High dose administration of IgG in a number of autoimmune diseases has a palliative effect that can be explained at least partially by saturation of FcRn-mediated protection of IgG, shortening the half-life of pathogenic IgG (see e.g., Jin & Balthasar, 2005, Hum. Immunol. 66:403-410; Akilesh et al., 2004, J. Clin. Invest. 113:1328-1333; Li et al., 2005, J. Clin. Invest. 115:3440-3450). Accordingly, specific blockade of FcRn-IgG interaction can be used to promote degradation of pathogenic IgG antibodies, for example to treat IgG mediated autoimmune diseases and to clear therapeutic antibodies from serum after administration. For example, in a rat model of experimentally-induced autoimmune myasthenia gravis, treatment with an FcRn heavy-chain specific monoclonal antibody resulted in a reduction of serum IgG concentration and a decrease in severity of the disease (Liu et al., 2007, J. Immunol. 178:5390-5398).

An absence of FcRn in hematopoietic cells is associated with more rapid clearance of IgG containing immune complexes from the bloodstream (Qiao et al., 2008, Proc. Natl. Acad. Sci. USA 105: 9337-9342). This indicates that specific blockade of FcRn-IgG interactions will also promote the clearance of IgG containing immune complexes from the circulation.

FcRn regulates the movement of IgG, and any bound cargo, between different compartments of the body via transcytosis across polarized cells. This process plays an important role in mucosal protection from infection, e.g., in the gastrointestinal tract. FcRn transports IgG across the epithelial cell barrier of the intestines and into the lumen. After IgG binds antigen in the lumen, the IgG/antigen complex is transported back through the barrier by FcRn into the lamina propria, allowing for processing of the IgG/antigen complex by dendritic cells and presentation of antigen to CD4⁺ T cells in regional lymph nodes.

FcRn also plays a critical role in MHC class II antigen presentation and MHC class I cross-presentation of IgG-complexed antigen. When antigen is presented as an IgG-containing immune complex (IC), dendritic cells that are CD8⁻CD11b⁺CD11c⁺ (inflammatory dendritic cells) display significant cross-presentation at low antigen doses in a pathway that is highly dependent upon FcRn expression. This pathway involves the internalization of the ICs by Fc7 receptors into an acidic endosome. Subsequent binding of the ICs by FcRn within antigen presenting cells (APCs) initiates specific mechanisms that result in trafficking of the antigen-bearing IC into compartments where antigen is processed into peptide epitopes compatible with loading onto MHC (see e.g., Baker et al., 2011, Proc. Natl. Acad. Sci. USA 108:9927-9932; Christianson et al., 2012, mAbs vol. 4, page 208-216). Thus, FcRn in DCs enhances MHC II antigen presentation and induces proliferation of antigen-specific CD4⁺ T-cells as well as exhibiting a fundamental role in antigen presentation to CD8⁺ T cells (cytotoxic T cells). This latter CD8⁺ T cell-pathway is called cross-presentation and involves the crossover of extracellular antigens into an MHC class I-dependent pathway.

Blockade of FcRn-Ig IC interaction inhibits antigen presentation of IC and subsequent T cell activation stimulated by immune-associated antigen presentation. Interactions with IgG IC in APCs such as DCs also promote secretion of inflammatory cytokines such as IL-12, IFNγ, and TNFα. Thus, blockade of FcRn-Ig IC interaction is useful to inhibit production of inflammatory cytokines by innate immune cells and antigen activated T cells.

FcRn contains a binding site for serum albumin that is distinct from its binding site for the Fc domain of IgG, due to ionic interactions between FcRn and IgG or albumin on opposite faces of the FcRn heavy chain (Chaudhury et al., 2006, Biochemistry 45:4983-4990). Like its binding to IgG, binding of FcRn to albumin is strongly pH-dependent, occurring at acidic pH (typically less than pH 6, and optimally at pH 5) but not at neutral pH. Similar to its role in protecting IgG from degradation, FcRn binding of albumin protects albumin from degradation and results in an extended serum half-life for albumin.

### Antibodies

In various embodiments described herein, antibodies or their antigen-biding domains are used in the design and preparation of agents that selectively bind FcRn and Fcγ receptors. The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of such are discussed below.

In a full-length antibody, each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. As used herein, a “hinge region” of an antibody is the flexible amino acid stretch in the central part of the heavy chains of the IgG and IgA immunoglobulin classes, which links these 2 heavy chains by disulfide bonds. The hinge region is located in between the C_{H}1 and C_{H}2 domains. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” or “antigen-binding fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)2 fragment, a bivalent fragment comprising two Fab fragments linked by a disulfide bridge at the hinge region; (iii) a Fd fragment consisting of the V_{H} and C_{H}1 domains; (iv) an Fv fragment consisting of the V_{L} and V_{H} domains of a single arm of an antibody, and (v) a dAb fragment (Ward et al., (1989) Nature 341:544-546, Winter et al., PCT publication WO 90/05144 A1 herein incorporated by reference), which comprises a single variable domain. CDRs can also be displayed on a non-immunoglobulin scaffold. Non-limiting examples of non-immunoglobulin antigen-binding scaffolds include a DARPIN, an affibody, an affilin, an adnectin, an affitin, an Obody or Obodies, a repebody, a fynomer, an alphabody, an avimer, an atrimer, a centyrin, a pronectin, an anticalin, a kunitz domain, or an Armadillo repeat protein. Examples of non-immunoglobulin antigen binding scaffolds are described in WO 2017/172981 and the tables therein, which are incorporated herein by reference.

Furthermore, although the two domains of the Fv fragment, V_{L} and V_{H}, are coded for by separate genes, they can be joined, using recombinant methods, by a synthetic linker that permits them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-54041354-5). In addition, single chain antibodies also include “linear antibodies” comprising a pair of tandem Fv segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870).

In a further aspect of this embodiment, the antibody can be a recombinant antibody, a monoclonal antibody, a chimeric antibody, a humanized antibody, or a human antibody.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., Nature 256: 495 (1975), or may be made by any of a wide variety of other recombinant DNA methods known to those of skill in the art (see e.g., U.S. Pat. No. 4,816,567).

Additional types of antibodies include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will ideally comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). The constant region, can if desired, include one or more modifications that modify or disrupt interaction of the human or humanized antibody with an Fc receptor, as described herein. Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

As used herein, “recombinant” antibody means any antibody whose production involves expression of a non-native DNA sequence encoding the desired antibody structure in an organism. As used herein, “affinity maturation” refers to the process by which antibodies are produced with increased affinity for antigen. With repeated exposures to the same antigen, a host or cell can produce antibodies of successively greater affinities.

Methods for designing and producing antibodies, including monoclonal, humanized, affinity-matured, or recombinant antibodies are well known in the art (see e.g., U.S. Pat. Nos. 8,663,980, 9,683,027, US 2018/0291101, WO 2011/015916, which are incorporated herein by herein in their entireties). To generate antibodies, conventional hybridoma techniques have been used in which clones of hybrid cells expressing genes coding for the light and heavy chains of an antibody molecule are obtained by immunization with an antigen molecule. This technique necessitates the fusion of cells of lymphocytic origin, containing the genes for antibody formation and cells forming immortal lines. The cells carrying the genes in question are generally obtained by random creation of libraries of circulating cells, and screening of the hybridomas with an antigen-antibody reaction after the hybridoma clones are multiplied and cultured. This technique can be uncertain and laborious with limited yield of antibodies, and is limited in application to non-human (e.g., mouse) antibody production.

In addition, monoclonal antibodies and their fragments can be expressed in various host systems, such as E. coli, yeast, and mammalian host cells. In general, a mammalian expression vector will contain (1) regulatory elements, usually in the form of viral promoter or enhancer sequences and characterized by a broad host and tissue range; (2) a “polylinker” sequence, facilitating the insertion of a DNA fragment within the plasmid vector; and (3) the sequences responsible for intron splicing and polyadenylation of mRNA transcripts. This contiguous region of the promoter-polylinker-polyadenylation site is commonly referred to as the transcription unit. The vector will likely also contain (4) a selectable marker gene(s) (e.g., the beta-lactamase gene), often conferring resistance to an antibiotic (such as ampicillin), allowing selection of initial positive transformants in E. coli; and (5) sequences facilitating the replication of the vector in both bacterial and mammalian hosts. Non-limiting examples of a mammalian expression vector include CDM8, pCMX, pAd/CMV/V5-DEST™, pAd/PL-DEST™, pCEP4, pOptiVEC™-TOPO™, pTracer™-SV40, pcDNA™3.2-DEST, pCMV⋅SPORT-βgal, pcDNA™3.3-TOPO™, pcDNA™3.4 TOPO™, or pcDNA™4/HisMax TOPO™. Expression of monoclonal antibodies behind a strong promoter increases the chances of identifying high-producing cell lines and obtaining higher yields of monoclonal antibodies. Consequently, Ig vectors with strong promoters are highly desirable for expressing any monoclonal antibody of interest. In addition, vectors with unique DNA cloning sites downstream of strong promoters have an added convenience.

Antibodies can be produced in bacteria, yeast, fungi, protozoa, insect cells, plants, or mammalian cells (see e.g., Frenzel et al. (2013) Front Immunol. 4: 217). A mammalian expression system is generally preferred for manufacturing most of therapeutic proteins, such as antibodies, as they require post-translational modifications. A variety of mammalian cell expression systems are now available for expression of antibodies, including but not limited to immortalized Chinese hamster ovary (CHO) cells, mouse myeloma (NSO), mouse L-cells, myeloma cell lines like J558L and Sp2/0, baby hamster kidney (BHK), or human embryo kidney (HEK-293).

### CDRs

As used herein, the term “Complementarity Determining Regions” (CDRs, i.e., CDR1, CDR2, and CDR3) refers to the amino acid residues of an antibody variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain). Likewise, “frameworks” (FWs) comprise amino acids 1-23 (FW1), 35-49 (FW2), 57-88 (FW3), and 98-107 (FW4) in the light chain variable domain and 1-30 (FW1), 36-49 (FW2), 66-94 (FW3), and 103-113 (FW4) in the heavy chain variable domain taking into account the Kabat numbering system (Kabat et al., Sequences of Proteins of Immunological Interest, 5th Ed. Public Health Service, National Institutes of Health, Bethesda, Md. (1987, 1991)).

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

Methods and computer programs for determining sequence similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

As used herein, “antibody variable domain” or “V_{H}/V_{L} domain pair” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain, which can be either a k light chain or a K light chain. As used herein, V_{K} refers to the variable domain (e.g., V_{L}) of a K light chain. Together, a V_{H}/V_{L} domain pair can bind and preferably and specifically bind an epitope on a given antigen.

In some embodiments as described herein, an antibody reagent is specific for a target and/or marker described herein (e.g., that binds specifically to and inhibits the target and/or marker). In some embodiments, an antibody reagent is a bispecific antibody construct that specifically binds FcRn and a type I or Type II Fc receptor, selected from the group consisting of CD32, CD32a, CD32b, CD32c, CD32a^{H}, CD32a^{R}, CD16, CD16a, CD16a_{V158}, CD16a^{F158}, CD16b, CD23, and DC-SIGN. Antibodies specific for type I or Type II Fc receptors are well known in the art (see e.g., U.S. Pat. No. 9,382,321; WO 2006/039418; US 2007/0253958; Chen et al. (2019) Ann. Rheum. Dis. 78:228-237; US 2016/0339115; Royen-Kerkhof et al. (2005) British Journal of Haematology 130: 130-137; Meyer et al. (2015) Blood 126(19): 2230-2238; Veri et al. (2007) Immunology 121: 392-404; U.S. Pat. No. 9,663,578; Bosque and Manning (2016) Autoimmunity Reviews 15: 1061-1068; US 2016/0185857; WO 2005/051999; U.S. Pat. No. 7,786,270; US 2015/0218275; U.S. Pat. No. 7,695,940; US 2007/0036786; U.S. Pat. No. 7,425,619; US 2008/0025913; WO 2018/039626 each of which is incorporated herein by reference in their entireties, especially with respect to any CDR or antibody sequences disclosed therein). Table 1 lists non-limiting examples of CDRs that can specifically bind to a type I or Type II Fc receptor, selected from the group consisting of CD32, CD32a, CD32b, CD32c, CD32aH, CD32aR, CD16, CD16a, CD16aV158, CD16aF158, CD16b, CD23, and DC-SIGN. Other examples of potential anti-Type I or anti-Type II Fc receptor CDRs are well known to those of skill in the art.

An antibody reagent or a V_{H}/V_{L} domain pair specific for a target and/or marker (e.g., a type I or Type II Fc receptor) described herein (e.g., that binds specifically to and inhibits the target and/or marker) can be an antibody reagent comprising one or more (e.g., one, two, three, four, five, or six) CDRs of any one of the antibodies recited in Table 1. In some embodiments of any of the aspects, an antibody reagent specific for a target and/or marker (e.g., a type I or Type II Fc receptor) described herein (e.g., that binds specifically to and inhibits the target and/or marker) can be an antibody reagent comprising the six CDRs of any one of the antibodies recited in Table 1. In some embodiments of any of the aspects, an antibody reagent specific for a target and/or marker (e.g., a type I or Type II Fc receptor) described herein (e.g., that binds specifically to and inhibits the target and/or marker) can be an antibody reagent comprising the three heavy chain CDRs of any one of the antibodies recited in Table 1. In some embodiments of any of the aspects, an antibody reagent specific for a target and/or marker (e.g., a type I or Type II Fc receptor) described herein (e.g., that binds specifically to and inhibits the target and/or marker) can be an antibody reagent comprising the three light chain CDRs of any one of the antibodies recited in Table 1. In some embodiments of any of the aspects, an antibody reagent specific for a target and/or marker (e.g., a type I or Type II Fc receptor) described herein (e.g., that binds specifically to and inhibits the target and/or marker) can be an antibody reagent comprising the V_{H} and/or V_{L} domains of any one of the antibodies recited in Table 1. In some embodiments of any of the aspects described herein, an antibody reagent specific for a target and/or marker (e.g., a type I or Type II Fc receptor) described herein (e.g., that binds specifically to and inhibits the target and/or marker) can be an antibody reagent comprising the V_{H} and V_{L} domains of any one of the antibodies recited in Table 1. Such antibody reagents are specifically contemplated for use in the methods and/or compositions described herein.

TABLE 1
Anti-Fc receptor antibody CDRs of interest

| Antibody & | | | | | | |
|---|---|---|---|---|---|---|
| Target | V_{H} CDR1 | V_{H} CDR2 | V_{H} CDR3 | V_{L} CDR1 | V_{L} CDR2 | V_{L} CDR3 |
| AT-10 | YYWMN | EIRJLKS | RDEYYA | RASESVD | GASNQGS | QQSKEVP |
| (Kabat); | (SEQ ID | NNYATHY | MDY | NFGISFM | (SEQ ID | WT |
| CD32a | NO: 1) | AESVKG | (SEQ ID | N | NO: 89) | (SEQ ID |
| | | (SEQ ID | NO: 45) | (SEQ ID | | NO: 113) |
| | | NO: 23) | | NO: 67) | | |
| IV.3 | NYGMN | WLNTYTG | GDYGYD | RSSKSLL | RMSVLAS | MQHLEYP |
| (Kabat); | (SEQ ID | ESIYPDD | DPLDY | HTNGNTY | (SEQ ID | LT |
| CD32a | NO: 2) | FKG | (SEQ ID | LH | NO: 90) | (SEQ ID |
| | | (SEQ ID | NO: 46) | (SEQ ID | | NO: 114) |
| | | NO: 24) | | NO: 68) | | |
| VIB9600; | NYGMN | WLNTYTG | GDYGYD | RSSKSLL | RMSVLAS | MQHLEYP |
| CD32a | (SEQ ID | ESWYPDD | DPLDY | HTNQNTY | (SEQ ID | LT |
| | NO: 3) | FKG | (SEQ ID | LH | NO: 91) | (SEQ ID |
| | | (SEQ ID | NO: 47) | (SEQ ID | | NO: 115) |
| | | NO: 25) | | NO: 69) | | |
| MDE-8 | SYGMH | VIWYDGS | DLGAAA | RASQGIN | DASSLES | QQFNSYP |
| (Kabat); | (SEQ ID | NYYYTDS | SDY | SALA | (SEQ ID | HT |
| CD32a | NO: 4) | VKG | (SEQ ID | (SEQ ID | NO: 92) | (SEQ ID |
| | | (SEQ ID | NO: 48) | NO: 70) | | NO: 116) |
| | | NO: 26) | | | | |
| hAT-10 | GFTFS | IRLKSNN | NRRDEY | ESVDNFG | GAS | QQSKEVP |
| (IMGT); | YYW | YAT | YAMDY | ISF | (SEQ ID | WT |
| CD32a | (SEQ ID | (SEQ ID | (SEQ ID | (SEQ ID | NO: 93) | (SEQ ID |
| | NO: 5) | NO: 27) | NO: 49) | NO: 71) | | NO: 117) |
| hIV.3.1e | GYTFT | LNTYTGE | ARGDYG | | | |
| (IMGT); | NYG | S | YDDPLD | | | |
| CD32a | (SEQ ID | (SEQ ID | Y | | | |
| | NO: 6) | NO: 28) | (SEQ ID | | | |
| | | | NO: 50) | | | |
| hIV.3.2b | | | | KSLLHTN | RMS | MQHLEYP |
| (IMGT); | | | | GNTY | (SEQ ID | LT |
| CD32a | | | | (SEQ ID | NO: 94) | (SEQ ID |
| | | | | NO: 72) | | NO: 118) |
| AT-10 | GFTFS | IRLKSNN | NRRDEY | ESVDNFG | GAS | QQSKEVP |
| (IMGT); | YYW | YAT | YAMDY | ISF | (SEQ ID | WT |
| CD32a | (SEQ ID | (SEQ ID | (SEQ ID | (SEQ ID | NO: 95) | (SEQ ID |
| | NO: 7) | NO: 29) | NO: 51) | NO: 73) | | NO: 119) |
| MDE-8 | GFTFS | IWYDGSN | ARDLGA | QGINSA | DAS | QQFNSYP |
| (IMGT); | SYG | Y | AASDY | (SEQ ID | (SEQ ID | HT |
| CD32a | (SEQ ID | (SEQ ID | (SEQ ID | NO: 74) | NO: 96) | (SEQ ID |
| | NO: 8) | NO: 30) | NO: 52) | | | NO: 120) |
| hIV.3.1c | | | | KSLLHTN | RMS | MQHLEYP |
| (IMGT); | | | | GNTY | (SEQ ID | LT |
| CD32a | | | | (SEQ ID | NO: 97) | (SEQ ID |
| | | | | NO: 75) | | NO: 121) |
| MDE-9; | SSTMH | LIGSGGG | GYFDWV | RASQGIS | AASSLQS | QQYNSYP |
| CD32 | (SEQ ID | IYYGDSV | DYFDY | SWLA | (SEQ ID | PT |
| | NO: 9) | KG | (SEQ ID | (SEQ ID | NO: 98) | (SEQ ID |
| | | (SEQ ID | NO: 53) | NO: 76) | | NO: 122) |
| | | NO: 31) | | | | |
| 2B6; | NYWIH | VIDPSDT | NGDSDY | RTSQSIG | NVSESIS | QQSNTWP |
| CD32b | (SEQ ID | YPNYNKK | YSGMDY | TNIH | (SEQ ID | FT |
| | NO: 10) | FKG | (SEQ ID | (SEQ ID | NO: 99); | (SEQ ID |
| | | (SEQ ID | NO: 54) | NO: 77) | YVSESIS | NO: 123) |
| | | NO: 32) | | | (SEQ ID | |
| | | | | | NO: 100); | |
| | | | | | or | |
| | | | | | YASESIS | |
| | | | | | (SEQ ID | |
| | | | | | NO: 101) | |
| GB3; | GYTFT | WIFPGTG | PFAY | RASQEIS | ATSALDS | LQYANYP |
| CD32b | DYYIY | NTYYNEN | (SEQ ID | GYLS | (SEQ ID | YT |
| | (SEQ ID | FKDKA | NO: 55) | (SEQ ID | NO: 102) | (SEQ ID |
| | NO: 11) | (SEQ ID | | NO: 78) | | NO: 124) |
| | | NO: 33) | | | | |
| 8A6; | DYYMA | SISYDGS | ARPGDY | RASQSVG | GASTRYT | LQYNNHP |
| CD32b | (SEQ ID | NKYYGDS | (SEQ ID | SYVD | (SEQ ID | YT |
| | NO: 12) | VKG | NO: 56) | (SEQ ID | NO: 103) | (SEQ ID |
| | | (SEQ ID | | NO: 79) | | NO: 125) |
| | | NO: 34) | | | | |
| Antibody | SYGIH | VIGYDGS | DQLGDA | KASQSVS | DASNRAT | QQRSNWP |
| 016; | (SEQ ID | DKNYADS | FDI | SSLA | (SEQ ID | PYT |
| CD32b | NO: 13) | VKG | (SEQ ID | (SEQ ID | NO: 104) | (SEQ ID |
| | | (SEQ ID | NO: 57) | NO: 80) | | NO: 126) |
| | | NO: 35) | | | | |
| Antibody | SYGIS | WISAYNG | DSAAHG | RASQGIS | AASSLQS | QQYNSYP |
| 020; | (SEQ ID | NTKYAQK | MDV | SWLA | (SEQ ID | YT |
| CD32b | NO: 14) | LQG | (SEQ ID | (SEQ ID | NO: 105) | (SEQ ID |
| | | (SEQ ID | NO: 58) | NO: 81) | | NO: 127) |
| | | NO: 36) | | | | |
| Antibody | SYGLS | WISPYNG | ASAAHG | RASQGIS | AASSLQS | QQYNSYP |
| 022; | (SEQ ID | NTHYAQK | MDV | SWLA | (SEQ ID | YT |
| CD32b | NO: 15) | LQG | (SEQ ID | (SEQ ID | NO: 106) | (SEQ ID |
| | | (SEQ ID | NO: 59) | NO: 82) | | NO: 128) |
| | | NO: 37) | | | | |
| Antibody | SYGLS | WISPYNG | DSAAHG | RASQGIS | AASSLQS | QQYNSYP |
| 024; | (SEQ ID | NTHYAQK | MDV | SWLA | (SEQ ID | YT |
| CD32b | NO: 16) | LQG | (SEQ ID | (SEQ ID | NO: 107) | (SEQ ID |
| | | (SEQ ID | NO: 60) | NO: 83) | | NO: 129) |
| | | NO: 38) | | | | |
| Antibody | SYGLS | WISAYNG | DSAAHG | RASQGIS | AASSLQS | QQYNSYP |
| 026; | (SEQ ID | NTNYAQK | MDV | SWLA | (SEQ ID | YT |
| CD32b | NO: 17) | LQG | (SEQ ID | (SEQ ID | NO: 108) | (SEQ ID |
| | | (SEQ ID | NO: 61) | NO: 84) | | NO: 130) |
| | | NO: 39) | | | | |
| Antibody | SYGIS | WISAYNG | DSAAHG | | | |
| 028; | (SEQ ID | NTKYAQK | MDV | | | |
| CD32b | NO: 18) | LQG | (SEQ ID | | | |
| | | (SEQ ID | NO: 62) | | | |
| | | NO: 40) | | | | |
| Antibody | NFVMS | GISGSGG | DSGGLF | RASQSVS | DASNRAT | QQRSNWP |
| 034; | (SEQ ID | NTDHADS | DY | SYLA | (SEQ ID | HLT |
| CD32b | NO: 19) | VKG | (SEQ ID | (SEQ ID | NO: 109) | (SEQ ID |
| | | (SEQ ID | NO: 63) | NO: 85) | | NO: 131) |
| | | NO: 41) | | | | |
| Antibody | TYGMH | VISHDGS | DQSIIE | RASQSVS | DASNRAT | QQRSNWG |
| 038; | (SEQ ID | DKYYADS | TFDY | SYLA | (SEQ ID | FT |
| CD32b | NO: 20) | VKG | (SEQ ID | (SEQ ID | NO: 110) | (SEQ ID |
| | | (SEQ ID | NO: 64) | NO: 86) | | NO: 132) |
| | | NO: 42) | | | | |
| Antibody | SYGMH | VIWYDGS | EGGRDA | RASQGIS | DASSLES | QQFNSYP |
| 053; | (SEQ ID | IKYYADS | FDI | SALA | (SEQ ID | HT |
| CD32b | NO: 21) | VKG | (SEQ ID | (SEQ ID | NO: 111) | (SEQ ID |
| | | (SEQ ID | NO: 65) | NO: 87) | | NO: 133) |
| | | NO: 43) | | | | |
| Antibody | SYAMS | AISDSGG | EIAVAL | RASQSVS | DASNRAT | QQRSSWP |
| 063; | (SEQ ID | STYYADS | FDY | SYLA | (SEQ ID | PYT |
| CD32b | NO: 22) | VKG | (SEQ ID | (SEQ ID | NO: 112) | (SEQ ID |
| | | (SEQ ID | NO: 66) | NO: 88) | | NO: 134) |
| | | NO: 44) | | | | |
| 3G8 or | TSGMG | HIWWDDD | INPAWF | KASQSVD | TTSNLES | QQSNEDP |
| GMA- 161; | VG | KRYNPAL | AY | FDGDSFM | (SEQ ID | YT |
| CD 16a or | (SEQ ID | KS | (SEQ ID | N | NO: 161) | (SEQ ID |
| CD 16b | NO: 135) | (SEQ ID | NO: 149) | (SEQ ID | | NO: 166) |
| | | NO: 142) | | NO: 156) | | |
| sdA1 | GFTFS | IYYSGGS | ARESID | | | |
| (D6); | NYG | T | Y | | | |
| CD 16a | (SEQ ID | (SEQ ID | (SEQ ID | | | |
| | NO: 136) | NO: 143) | NO: 150) | | | |
| sdA2 | GFTFS | VNHSGGS | ARVGSF | | | |
| (E11); | SYG | T | DF | | | |
| CD 16a | (SEQ ID | (SEQ ID | (SEQ ID | | | |
| | NO: 137) | NO: 144) | NO: 151) | | | |
| 6G5; | SSNWW | RISGSGG | DWAQIA | TGTSDDV | DVAKRAS | CSYTTSS |
| CD23 | T | ATNYNPS | GTTLGF | GGYNYVS | (SEQ ID | TLL |
| | (SEQ ID | L | (SEQ ID | (SEQ ID | NO: 162) | (SEQ ID |
| | NO: 138) | (SEQ ID | NO: 152) | NO: 157) | | NO: 167) |
| | | NO: 145) | | | | |
| 5E8; | FNNYY | RISSSGD | LTTGSD | RASQDIR | VASSLQS | LQVYSTP |
| CD23 | MD | PTWYADS | S | YYLN | (SEQ ID | RT |
| | (SEQ ID | V | (SEQ ID | (SEQ ID | NO: 163) | (SEQ ID |
| | NO: 139) | (SEQ ID | NO: 153) | NO: 158) | | NO: 168) |
| | | NO: 146) | | | | |
| DC-SIGN | NYYIH | WIFPGNF | YGYAVD | KASQDVS | SASYRYT | QQHYITP |
| | (SEQ ID | KTEYNEK | Y | TA | (SEQ ID | LT |
| | NO: 140) | FKG | (SEQ ID | (SEQ ID | NO: 164) | (SEQ ID |
| | | (SEQ ID | NO: 154) | NO: 159) | | NO: 169) |
| | | NO: 147) | | | | |
| DC-SIGN | DTYMH | RIDPANG | YYGIYV | KSSQSLL | LVSKLDS | WQDTHFP |
| | (SEQ ID | NTKYDPK | DY | DSDGKTY | (SEQ ID | HV |
| | NO: 141) | FQG | (SEQ ID | LN | NO: 165) | (SEQ ID |
| | | (SEQ ID | NO: 155) | (SEQ ID | | NO: 170) |
| | | NO: 148) | | NO: 160) | | |

In some embodiments, an antibody reagent is a bispecific antibody construct that specifically binds FcRn and a type I or Type II Fc receptor. Antibodies specific for FcRn are well known in the art (see e.g., US Patent Publication No. 2018/0291101; US 2016/0264668; each of which is incorporated herein by reference in their entireties, especially with respect to any CDR or antibody sequences disclosed therein that specifically bind FcRn). Table 2 lists non-limiting examples of CDRs that can specifically bind to FcRn. Other examples of potential anti-FcRn CDRs are well known to those of skill in the art. An antibody reagent or a V_{H}/V_{L} domain pair specific for a target and/or marker (e.g., FcRn) described herein (e.g., that binds specifically to and inhibits the target and/or marker) can be an antibody reagent comprising one or more (e.g., one, two, three, four, five, or six) CDRs of any one of the antibodies recited in Table 2. In some embodiments of any of the aspects, an antibody reagent specific for a target and/or marker (e.g., FcRn) described herein (e.g., that binds specifically to and inhibits the target and/or marker) can be an antibody reagent comprising the six CDRs of any one of the antibodies recited in Table 2. In some embodiments of any of the aspects, an antibody reagent specific for a target and/or marker (e.g., FcRn) described herein (e.g., that binds specifically to and inhibits the target and/or marker) can be an antibody reagent comprising the three heavy chain CDRs of any one of the antibodies recited in Table 2. In some embodiments of any of the aspects, an antibody reagent specific for a target and/or marker (e.g., FcRn) described herein (e.g., that binds specifically to and inhibits the target and/or marker) can be an antibody reagent comprising the three light chain CDRs of any one of the antibodies recited in Table 2. In some embodiments of any of the aspects, an antibody reagent specific for a target and/or marker (e.g., FcRn) described herein (e.g., that binds specifically to and inhibits the target and/or marker) can be an antibody reagent comprising the V_{H} and/or V_{L} domains of any one of the antibodies recited in Table 2. In some embodiments of any of the aspects described herein, an antibody reagent specific for a target and/or marker (e.g., FcRn) described herein (e.g., that binds specifically to and inhibits the target and/or marker) can be an antibody reagent comprising the V_{H} and V_{L} domains of any one of the antibodies recited in Table 2. Such antibody reagents are specifically contemplated for use in the methods and/or compositions described herein.

TABLE 2
Anti-FcRn antibody CDRs of interest

| Antibody | V_{H} CDR1 | V_{H} CDR2 | V_{H} CDR3 | V_{L} CDR1 | V_{L} CDR2 | V_{L} CDR3 |
|---|---|---|---|---|---|---|
| SYNT001 | SYGIS | EIYPRSG | STTVSPA | KASDHIN | GATSLET | NTYGNN |
| | (SEQ ID | NTYYNE | DF (SEQ | NWLA | (SEQ ID | PHT (SEQ |
| | NO: 171) | KFK (SEQ | ID NO: | (SEQ ID | NO: 194) | ID NO: |
| | | ID NO: | 175) | NO: 192) | | 197) |
| | | 173) | | | | |
| | | | STTVSPP | | | HQYYNT |
| | | | PI (SEQ | | | PYT (SEQ |
| | | | ID NO: | | | ID NO: |
| | | | 176) | | | 198) |
| | | | STTVSPP | | | HQYYSTP |
| | | | AH (SEQ | | | YT (SEQ |
| | | | ID NO: | | | ID NO: |
| | | | 177) | | | 199) |
| | | | STTVAPP | | | QQYYSTP |
| | | | RL (SEQ | | | YT (SEQ |
| | | | ID NO: | | | ID NO: |
| | | | 178) | | | 200) |
| | | | STTVHPD | | | |
| | | | RN (SEQ | | | |
| | | | ID NO: | | | |
| | | | 179) | | | |
| | | | STTVSPP | | | |
| | | | AL (SEQ | | | |
| | | | ID NO: | | | |
| | | | 180) | | | |
| | | | STTVHPD | | | |
| | | | HN (SEQ | | | |
| | | | ID NO: | | | |
| | | | 181) | | | |
| | | | STTVSPP | | | |
| | | | HL(SEQ | | | |
| | | | ID NO: | | | |
| | | | 182) | | | |
| | | | STTVAPP | | | |
| | | | PL (SEQ | | | |
| | | | ID NO: | | | |
| | | | 183) | | | |
| | | | STTVSPP | | | |
| | | | HL (SEQ | | | |
| | | | ID NO: | | | |
| | | | 184) | | | |
| | | | STTVAPP | | | |
| | | | GH (SEQ | | | |
| | | | ID NO: | | | |
| | | | 185) | | | |
| | | | STTVSPP | | | |
| | | | RV (SEQ | | | |
| | | | ID NO: | | | |
| | | | 186) | | | |
| | | | STTVSPP | | | |
| | | | PL (SEQ | | | |
| | | | ID NO: | | | |
| | | | 187) | | | |
| | | | STTVAPP | | | |
| | | | AH (SEQ | | | |
| | | | ID NO: | | | |
| | | | 188) | | | |
| | | | STTVRPP | | | |
| | | | GI (SEQ | | | |
| | | | ID NO: | | | |
| | | | 189) | | | |
| | | | STTVSAP | | | |
| | | | GV (SEQ | | | |
| | | | ID NO: | | | |
| | | | 190) | | | |
| 1638 | GFSLSTY | NIWWDD | TPAYYGS | RTSEDIY | VAKTLQ | LQGFKFP |
| | GVGVG | DKRYNPS | HPPFDY | TNL | AD (SEQ | WT (SEQ |
| | (SEQ ID | LEN (SEQ | (SEQ ID | (SEQ ID | ID NO: | ID NO: |
| | NO: 172) | ID NO: | NO: 191) | NO: 193) | 195), or | 201) |
| | | 174) | | | VAKTLQ | |
| | | | | | E (SEQ | |
| | | | | | ID NO: | |
| | | | | | 196) | |

### Antibody Modifications

The term “Fe region” is used to define the C-terminal region of an immunoglobulin heavy chain, which may be generated by papain digestion of an intact antibody. The Fc region can be a native sequence Fc region or a variant Fc region. The Fc region of an immunoglobulin generally comprises two constant domains, a C_{H}2 domain and a C_{H}3 domain, and optionally comprises a C_{H}4 domain. Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases, these effector functions are desirable for therapeutic antibodies but in other cases might be unnecessary or even deleterious, depending on the therapeutic objectives.

The antibody compositions herein, as well as the antibodies used in the methods and uses described herein, can be “effector-deficient.” As used herein, an “effector-deficient” antibody is defined as an antibody having an Fc region that has been altered so as to reduce or eliminate Fc-binding to CD16, CD16a, CD16a^{V158}, CD16a^{F158}, CD16b, CD32, CD32a, CD32b, CD32c, CD23, DC-SIGN, and/or FcRn Fc receptors. A non-limiting example of mutations that reduce Fc-binding to CD16, CD32, and CD64 include E233P, L234A, L235A, G237M, D265A, D265N, E269R, D270A, D270N, N297A, N297Q, N297D, N297R, S298N, T299A, or any combinations thereof (numbering is EU index of Kabat). A non-limiting example of mutations that reduce Fc-binding to FcRn include I253A, H310A, H435A, or any combinations thereof (numbering is EU index of Kabat). An effector-deficient antibody may have one or more of the aforementioned mutations, or any combinations thereof.

The antibody compositions herein, as well as the antibodies used in the methods and uses described herein, can be mutated to increased their circulating half-life. In some embodiments, the Fc region can comprise mutations that enhance FcRn binding to the Fc region, in order to extend the half-life of these medications. Non-limiting examples of half-life-enhancing mutations include M252Y, S254T, T256E, ΔE294, G385D, Q386P, N389S, M428L, H433K, N434F, N434S, Y436H, or any combination thereof (see e.g., U.S. Pat. No. 8,323,962; Zalevsky et al. (2010) Nat. Biotechnol. 28(2): 157-159; Bas et al. (2019 Jan. 25) J. Immunol., “Fe Sialylation Prolongs Serum Half-Life of Therapeutic Antibodies”). An antibody as described herein may have one or more of the aforementioned half-life-enhancing mutations, or any combinations thereof.

In one embodiment, the reduction in Fc-binding to Fc receptors is a complete reduction as compared to an effector-competent control. In other aspects, the reduction in about 50%, about 60%, about 70%, about 80%, about 90%, or about 95%, or more, as compared to an effector-competent antibody control. Methods for determining whether an antibody has a reduced Fc-binding to CD16, CD32, CD64 and/or FcRn are well known in the art (see e.g., US 2011/0212087 A1, WO 2013/165690, U.S. Pat. No. 9,382,321 B2, US 2018/0291101 A1, and Vafa O. et al. “An engineered Fc variant of an IgG eliminates all immune effector functions via structural perturbations” (January 2014) Methods 65:114; PubMed ID: 23872058).

In some embodiments of any of the aspects, the immunoglobulin constant region can include a C_{H}3 C-terminal lysine deletion (ΔK445) (Lys0) and or an S226P mutation to stabilize the hinge region.

### Bispecific Antibodies

The term “bispecific antibody” or “bispecific antibody construct” refers to an antibody having the capacity to bind to two distinct epitopes either on a single antigen or two different antigens (see e.g., WO 2014/209804; Brinkmann and Kontermann (2017) MAbs 9(2): 182-212, especially FIG. 2 “The zoo of bispecific antibody formats;” incorporated herein by reference in their entireties). As used herein, “epitope” or “antigenic determinant” refers to a site on an antigen to which an antibody binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping is surface plasmon resonance.

In some embodiments, a bispecific antibody construct contains more than one antigen-binding domain for each antigen. For example, additional V_{H} and V_{L} domains can be fused to the N-terminus of the V_{H} and V_{L} domains of an existing antibody, effectively arranging the antigen-binding sites in tandem. These types of antibodies are known as dual-variable-domain antibodies (DvD-Ig) (Tarcsa, E. et al. In: Bispecific Antibodies. Kontermann R E (ed.), Springer Heidelberg Dordrecht London New York, pp. 171-185 (201 1)). One advantage of the DvD-Ig format is that the respective V_{H}/V_{L} domain pairs can only associate with their cognate partners, as opposed to the random assortment of V_{H} and V_{L} domains that can occur in some other bispecific formats. In the DvD-Ig format, only cognate V_{H}/V_{L} pairs will form, and all such pairs will be competent to bind their respective antigens. DvD-Ig design and production is well known in the art (see e.g., U.S. Pat. No. 7,612,181, which is incorporated herein by reference in its entirety). As a non-limiting example, a DvD-Ig format bispecific antibody construct can be produced by inserting V_{H1}-V_{H2} and V_{L1}-V_{L2} domains into a DvD-Ig vector, such as pPBTAK21 (IgG1-Fcmut_VL+Kappa-Ex.c) or a similar vector to pPBTAK21 with IgG4-Fcmut domains instead of IgG1-Fcmut domains.

In some embodiments, the V_{H} of the first V_{H}/V_{L} domain pair is joined to the V_{H} of the second V_{H}/V_{L} domain pair by a linker (e.g., V_{H1}-V_{H2}) and the V_{L} of the first V_{H}/V_{L} domain pair is joined to the V_{L} of the second V_{H}/V_{L} domain pair by a linker (e.g., V_{L}1-V_{L}2). The linker can be a chemical linker or a polypeptide linker. The linker can be a “short linker” or a “long linker”. Non-limiting examples of a short linker include GGSGGGGSG (SEQ ID NO: 202) and TVAAP (SEQ ID NO: 203). Non-limiting examples of a long linker include GGSGGGGSGGGGS (SEQ ID NO: 204) and TVAAPSVFIFPP (SEQ ID NO: 205). Linkers for DvD-Ig antibody constructs are well-known in the art (see e.g., U.S. Pat. No. 7,612,181, incorporated herein by reference in its entirety). Linkers can also be selected from the group consisting of AKTTPKLEEGEFSEAR (SEQ ID NO: 206); AKTTPKLEEGEFSEARV (SEQ ID NO: 207); AKTTPKLGG (SEQ ID NO: 208); SAKTTPKLGG; (SEQ ID NO: 209); SAKTTP (SEQ ID NO: 210); RADAAP (SEQ ID NO: 211); RADAAPTVS (SEQ ID NO: 212); RADAAAAGGPGS (SEQ ID NO: 213); RADAAAA(G4S)4 (SEQ ID NO: 214); SAKTTPKLEEGEFSEARV (SEQ ID NO: 215); ADAAP (SEQ ID NO: 216); ADAAPTVSIFPP (SEQ ID NO: 217); TVAAP (SEQ ID NO: 203); TVAAPSVFIFPP (SEQ ID NO: 205); QPKAAP (SEQ ID NO: 218); QPKAAPSVTLFPP (SEQ ID NO: 219); AKTTPP (SEQ ID NO: 220); AKTTPPSVTPLAP (SEQ ID NO: 221); AKTTAP (SEQ ID NO: 222); AKTTAPSVYPLAP (SEQ ID NO: 223); ASTKGP (SEQ ID NO: 224); ASTKGPSVFPLAP (SEQ ID NO: 225); GGGSGGGGSGGGGS (SEQ ID NO: 226); GENKVEYAPALMALS (SEQ ID NO: 227); GPAKELTPLKEAKVS (SEQ ID NO: 228); and GHEAAAVMQVQYPAS (SEQ ID NO: 229). The linker chosen for joining the V_{H} of the first V_{H}/V_{L} domain pair to the V_{H} of the second V_{H}/V_{L} domain pair can be the same or different as the linker chosen for joining the V_{L} of the first V_{H}/V_{L} domain pair to the V_{L} of the second V_{H}/V_{L} domain pair.

As described herein, the length of the linker can influence the distance between the first V_{H}/V_{L} domain pair and the second V_{H}/V_{L} domain pair. In some embodiments, the linker positions the first V_{H}/V_{L} domain pair a distance of about 10 Å-100 Å (e.g., of about 10 Å, about 11 Å, about 12 Å, about 13 Å, about 14 Å, about 15 Å, about 16 Å, about 17 Å, about 18 Å, about 19 Å, about 20 Å, about 21 Å, about 22 Å, about 23 Å, about 24 Å, about 25 Å, about 26 Å, about 27 Å, about 28 Å, about 29 Å, about 30 Å, about 31 Å, about 32 Å, about 33 Å, about 34 Å, about 35 Å, about 36 Å, about 37 Å, about 38 Å, about 39 Å, about 40 Å, about 41 Å, about 42 Å, about 43 Å, about 44 Å, about 45 Å, about 46 Å, about 47 Å, about 48 Å, about 49 Å, about 50 Å, about 51 Å, about 52 Å, about 53 Å, about 54 Å, about 55 Å, about 56 Å, about 57 Å, about 58 Å, about 59 Å, about 60 Å, about 61 Å, about 62 Å, about 63 Å, about 64 Å, about 65 Å, about 66 Å, about 67 Å, about 68 Å, about 69 Å, about 70 Å, about 71 Å, about 72 Å, about 73 Å, about 74 Å, about 75 Å, about 76 Å, about 77 Å, about 78 Å, about 79 Å, about 80 Å, about 81 Å, about 82 Å, about 83 Å, about 84 Å, about 85 Å, about 86 Å, about 87 Å, about 88 Å, about 89 Å, about 90 Å, about 91 Å, about 92 Å, about 93 Å, about 94 Å, about 95 Å, about 96 Å, about 97 Å, about 98 Å, about 99 Å, or about 100 Å) away from the second V_{H}/V_{L} domain pair. In some embodiments, the linker positions the first V_{H}/V_{L} domain pair a distance of about 41 Å away from the second V_{H}/V_{L} domain pair. In some embodiments, the distance between the first V_{H}/V_{L} domain pair and the second V_{H}/V_{L} domain pair can mimic the distance between CD32a and FcRn bound to immunocomplexed antibody (see e.g., FIG. 4A, FIG. 4B)

In some embodiments, the first V_{H}/V_{L} domain pair is on the amino terminus of the bispecific antibody construct. In other embodiments, the second V_{H}/V_{L} domain pair is on the amino terminus of the bispecific antibody construct. As a non-limiting example, an anti-CD32a V_{H}/V_{L} domain pair can be on the amino-terminus of a bispecific antibody construct, attached by their carboxyl-termini to an anti-FcRn V_{H}/V_{L} domain pair. As another non-limiting example, an anti-FcRn V_{H}/V_{L} domain pair can be on the amino-terminus of a bispecific antibody construct, attached by their carboxyl-termini to an anti-CD32a V_{H}/V_{L} domain pair.

Bispecific antibodies can be produced via biological methods, such as somatic hybridization; or genetic methods, such as the expression of a non-native DNA sequence encoding the desired antibody structure in an organism; chemical methods, such as chemical conjugation of two antibodies; or a combination thereof (see e.g., Kontermann, R. E. In: Bispecific Antibodies. Kontermann R E (ed.), Springer Heidelberg Dordrecht London New York, pp. 1-28 (201 1)).

Chemically conjugated bispecific antibodies arise from the chemical coupling of two existing antibodies or antibody fragments. Typical couplings include cross-linking two different full-length antibodies, cross-linking two different Fab′ fragments to produce a bispecific F(ab′)2, and cross-linking a F(ab′)2 fragment with a different Fab′ fragment to produce a bispecific F(ab′)3. For chemical conjugation, oxidative reassociation strategies can be used. Current methodologies include the use of the homo- or heterobifunctional cross-linking reagents (Id.).

Heterobifunctional cross-linking reagents have reactivity toward two distinct reactive groups on, for example, antibody molecules. Examples of heterobifunctional cross-linking reagents include SPDP (N-succinimidyl-3-(2-pyridyldithio)propionate), SATA (succinimidyl acetylthioacetate), SMCC (succinimidyl trans-4-(maleimidylmethyl) cyclohexane-1-carboxylate), EDAC (1-ethyl-3-(3-dimethylaminopropyl) carbodiimide), PEAS (N-((2-pyridyldithio)ethyl)-4-azidosalicylamide), ATFB, SE (4-azido-2,3,5,6-tetrafluorobenzoic acid, succinimidyl ester), benzophenone-4-maleimide, benzophenone-4-isothiocyanate, 4-benzoylbenzoic acid, succinimidyl ester, iodoacetamide azide, iodoacetamide alkyne, Click-iT maleimide DIBO alkyne, azido (PEO)4 propionic acid, succinimidyl ester, alkyne, succinimidyl ester, Click-iT succinimidyl ester DIBO alkyne, Sulfo-SBED (Sulfo-N-hydroxysuccinimidyl-2-(6-[biotinamido]-2-(p-azido benzamido)-hexanoamido) ethyl-1,3′-dithioproprionate), photoreactive amino acids {e.g., L-Photo-Leucine and L-Photo-Methionine), NHS-haloacetyl crosslinkers such as, for example, Sulfo-SIAB, SIAB, SBAP, SIA, NHS-maleimide crosslinkers such as, for example, Sulfo-SMCC, SM(PEG)n series crosslinkers, SMCC, LC-SMCC, Sulfo-EMCS, EMCS, Sulfo-GMBS, GMBS, Sulfo-KMUS, Sulfo-MBS, MBS, Sulfo-SMPB, SMPB, AMAS, BMPS, SMPH, PEG12-SPDP, PEG4-SPDP, Sulfo-LC-SPDP, LC-SPDP, SMPT, DCC (N, N′-Dicyclohexylcarbodiimide), EDC (1-Ethyl-3-(3-dimethylaminopropyl) carbodiimide), NHS (N-hydroxysuccinimide), Sulfo-NHS (N-hydroxysulfosuccinimide), BMPH, EMCH, KMUH, MPBH, PDPH, and PMPI.

Homobifunctional cross-linking reagents have reactivity toward the same reactive group on a molecule, for example, an antibody. Examples of homobifunctional cross-linking reagents include DTNB (5,5′-dithiobis(2-nitrobenzoic acid), o-PDM (0-phenylenedimaleimide), DMA (dimethyl adipimidate), DMP (dimethyl pimelimidate), DMS (dimethyl suberimidate), DTBP (dithiobispropionimidate), BS(PEG)5, BS(PEG)9, BS3, BSOCOES, DSG, DSP, DSS, DST, DTSSP, EGS, Sulfo-EGS, TSAT, DFDNB, BM(PEG)n crosslinkers, BMB, BMDB, BMH, BMOE, DTME, and TMEA.

Somatic hybridization is the fusion of two distinct hybridoma (a fusion of B cells that produce a specific antibody and myeloma cells) cell lines, producing a quadroma capable of generating two different antibody heavy (VHA and VHB) and light chains (VLA and VLB). (Kontermann, R. E. In: Bispecific Antibodies. Kontermann R E (ed.), Springer Heidelberg Dordrecht London New York, pp. 1-28 (201 1)). These heavy and light chains combine randomly within the cell, resulting in bispecific antibodies (a VHA combined with a VLA and a VHB combined with a VLB), as well as some nonfunctional (e.g. two VHAs combined with two VLBs) and monospecific (two VHAs combined with two VLAs) antibodies. The bispecific antibodies can then be purified using, for example, two different affinity chromatography columns. Similar to monospecific antibodies, bispecific antibodies can also contain an Fc region that elicits Fc-mediated effects downstream of antigen binding. These effects can be reduced by, for example, proteolytically cleaving the Fc region from the bispecific antibody by pepsin digestion, resulting in bispecific F(ab′)2 molecules (Id.).

Bispecific antibodies can also be generated via genetic means, e.g., in vitro expression of a plasmid containing a DNA sequence corresponding to the desired antibody structure. See, e.g., Kontermann, R. E. In: Bispecific Antibodies. Kontermann R E (ed.), Springer Heidelberg Dordrecht London New York, pp. 1-28 (201 1). Such bispecific antibodies are discussed in greater detail below.

A bispecific antibody can be bivalent, trivalent, or tetravalent. As used herein, “valent”, “valence”, “valencies”, or other grammatical variations thereof, mean the number of antigen binding sites in an antibody molecule or construct. These antigen recognition sites may recognize the same epitope or different epitopes. Bivalent and bispecific molecules are described in, e.g., Kostelny et al. (1992) J Immunol 148:1547, Pack and Pluckthun (1992) Biochemistry 31: 1579, Hollinger et al., 1993, supra, Gruber et al. (1994) J Immunol:5368, Zhu et al. (1997) Protein Sci 6:781, Hu et al. (1996) Cancer Res. 56:3055, Adams ef a/. (1993) Cancer Res. 53:4026, and McCartney, et al. (1995) Protein Eng. 8:301. Trivalent bispecific antibodies and tetravalent bispecific antibodies are also known in the art. See, e.g., Kontermann R E (ed.), Springer Heidelberg Dordrecht London New York, pp. 199-216 (201 1). A bispecific antibody can also have valencies higher than 4. Such antibodies can be generated by, for example, dock and lock conjugation method. (Chang, C.-H. et al. In: Bispecific Antibodies. Kontermann R E (ed.), Springer Heidelberg Dordrecht London New York, pp. 199-216 (201 1)).

Recombinant antibodies include tandem scFv (taFv or scFv₂), diabody, dAb2/VHH2, knob-into-holes derivatives, SEED-IgG, heteroFc-scFv, Fab-scFv, scFv-Jun/Fos, Fab′-Jun/Fos, tribody, DNL-F(ab)3, scFv3-CH1/CL, Fab-scFv2, IgG-scFab, IgG-scFv, scFv-IgG, scFv2-Fc, F(ab′)2-scFv₂, scDB-Fc, scDb-CH3, Db-Fc, scFv2-H/L, DVD-Ig, tandAb, scFv-dhlx-scFv, dAb2-lgG, dAb-IgG, dAb-Fc-dAb, and combinations thereof.

Variable regions of antibodies are typically isolated as single-chain Fv (scFv) or Fab fragments. ScFv fragments are composed of V_{H} and V_{L} domains linked by a short 10-25 amino acid linker. Once isolated, scFv fragments can be genetically linked with a flexible peptide linker such as, for example, one or more repeats of Ala-Ala-Ala, Gly-Gly-Gly-Gly-Ser, etc. The resultant peptide, a tandem scFv (taFv or scFv₂) can be arranged in various ways, with V_{H}-V_{L} or V_{L}-V_{H} ordering for each scFv of the taFv. (Kontermann, R. E. In: Bispecific Antibodies. Kontermann R E (ed.), Springer Heidelberg Dordrecht London New York, pp. 1-28 (201 1)).

Bispecific diabodies are another form of antibody fragment. In contrast to taFvs, diabodies are composed of two separate polypeptide chains from, for example, antibodies A and B, each chain bearing two variable domains (V_{H}A-V_{L}B and V_{H}B-V_{L}A or V_{L}A-V_{H}B and V_{L}B-V_{H}A). The linkers joining the variable domains are short (about five amino acids), preventing the association of V_{H} and V_{L} domains on the same chain, and promoting the association of V_{H} and V_{L} domains on different chains. Heterodimers that form are functional against both target antigens, (such as, e.g., V_{H}A-V_{L}B with V_{H}B-V_{L}A or V_{L}A-V_{H}B with V_{L}B-V_{H}A), however, homodimers can also form (such as, e.g., V_{H}A-V_{L}B with V_{H}A-V_{L}B, V_{H}B-V_{L}A with V_{H}B-V_{L}A, etc.), leading to nonfunctional molecules. Several strategies exist to prevent homodimerization, including the introduction of disulfide bonds to covalently join the two polypeptide chains, modification of the polypeptide chains to include large amino acids on one chain and small amino acids on the other (knobs-into-holes structures, discussed below), and addition of cysteine residues at C-terminal extensions. Another strategy is to join the two polypeptide chains by a linker sequence, producing a single-chain diabody molecule (scDb) that exhibits a more compact structure than a taFv. ScDbs or Dbs can be also be fused to the IgG1 C_{H}3 domain or the Fc region, producing di-diabodies. Examples of di-diabodies include, but are not limited to, scDb-Fc, Db-Fc, scDb-Chi3, and Db-Chi3. Additionally, scDbs can be used to make tetravalent bispecific molecules. By shortening the linker sequence of scDbs from about 15 amino acids to about 5 amino acids, dimeric single-chain diabody molecules result, known as TandAbs (Muller, D. and Kontermann, R. E. In: Bispecific Antibodies. Kontermann R E (ed.), Springer Heidelberg Dordrecht London New York, pp. 83-100 (201 1)).

Yet another strategy for generating a bispecific antibody includes fusing heterodimerizing zinc peptides to the C-termini of the antibody molecules (scFvs or Fabs). A non-limiting example of this strategy is the use of antibody fragments linked to jun-fos leucine zippers (e.g. scFv-Jun/Fos and Fab′-Jun/Fos).

An additional method for generating a bispecific antibody molecule includes derivatizing two antibodies with different antigen binding moieties with biotin and then linking the two antibodies via streptavidin, followed by purification and isolation of the resultant bispecific antibody.

Constant immunoglobulin domains can also be used to promote heterodimerization of two polypeptide chains (IgG-like antibodies, discussed below). Non-limiting examples of this type of approach to making a bispecific antibody include the introduction of knobs-into-holes structures into the two polypeptides and utilization of the naturally occurring heterodimerization of the C_{L} and C_{H} domains (Kontermann, R. E. In: Bispecific Antibodies. Kontermann R E (ed.), Springer Heidelberg Dordrecht London New York, pp. 1-28 (201 1)).

Additional types of bispecific antibodies include those that contain more than one antigen-binding site for each antigen. As described previously, additional V_{H} and V_{L} domains can be fused to the N-terminus of the V_{H} and V_{L} domains of an existing antibody, effectively arranging the antigen-binding sites in tandem. The DvD-Ig format discussed above is an example that positions two different antigen-binding domains on each arm of an Ig construct. If so desried, additional binding domains can be added to the N-terminal end of the constructs. (see e.g., Tarcsa, E. et al. In: Bispecific Antibodies. Kontermann R E (ed.), Springer Heidelberg Dordrecht London New York, pp. 171-185 (201 1)). Yet another method for producing antibodies that contain more than one antigen-binding site for an antigen is to fuse scFv fragments to the N-terminus of the heavy chain or the C-terminus of the light chain (discussed further below).

Because the majority of antibodies approved for therapy have been IgG or IgG-like, one embodiment the bispecific construct as described herein can be engineered to be IgG-like, to the extent that they can have an Fc domain. Similar to diabodies that require heterodimerization of engineered polypeptide chains, IgG-like antibodies also require heterodimerization to prevent the interaction of like heavy chains or heavy chains and light chains from two antibodies of different specificity (see e.g., Jin, P. and Zhu, Z. In: Bispecific Antibodies. Kontermann R E (ed.), Springer Heidelberg Dordrecht London New York, pp. 151-169 (201 1)).

So-called “knobs-into-holes” structures facilitate heterodimerization of polypeptide chains by introducing large amino acids (knobs) into one chain of a desired heterodimer and small amino acids (holes) into the other chain of the desired heterodimer. Steric interactions will favor the interaction of the knobs with holes, rather than knobs with knobs or holes with holes. In the context of bispecific IgG-like antibodies, like heavy chains can be prevented from homodimerizing by the introduction of knobs-into-holes structures into the C_{H}3 domain of the Fc region. Similarly, promoting the interaction of heavy chains and light chains specific to the same antigen can be accomplished by engineering knobs-into-holes structures at the V_{H}-V_{L} interface. Other examples of knobs-into-holes structures exist and the examples discussed above should not be construed to be limiting. Other methods to promote heterodimerization of Fc regions include engineering charge polarity into Fc domains (see e.g., Gunasekaran et al., 2010) and SEED technology (SEED-IgG) (see e.g., Davis et al, 2010).

Additional heterodimerized IgG-like antibodies include, but are not limited to, heteroFc-scFvs, Fab-scFvs, IgG-scFv, and scFv-IgG. HeteroFc-scFvs link two distinct scFvs to heterodimerizable Fc domains while Fab-scFvs contain a Fab domain specific to one epitope linked to an scFv specific to a different epitope. IgG-scFv and scFv-IgG are Ig-like antibodies that have scFvs linked to their C-termini and N-termini, respectively (Kontermann, R. E. In: Bispecific Antibodies. Kontermann R E (ed.), Springer Heidelberg Dordrecht London New York, pp. 151-169 (201 1)).

Though most naturally occurring antibodies are composed of heavy chains and light chains, camelids (e.g. camels, dromedaries, llamas, and alpacas) and some sharks produce antibodies that consist only of heavy chains. These antibodies bind antigenic epitopes using a single variable domain known as V_{H}H. When produced in Escherichia coli, these molecules are termed single domain antibodies (dAbs). The simplest application of dAbs in bispecific antibodies is to link two different dAbs together to form dAb2S (V_{H}H2s). dAbs can also be applied to IgG-like bispecific antibodies. Examples of this include, but are not limited to, dAb2-IgGs, dAb-IgGs, and dAb-Fc-dAbs. dAb2-IgGs have a similar structure to intact antibodies, but with dAbs linked to the N-terminal end of the molecule. dAb-IgGs are intact antibodies specific for one epitope with a single dAb specific for another epitope linked to the N-termini or C-termini of the heavy chains. Lastly, dAb-Fc-dAbs are Fc domains with dAbs specific for one epitope linked to the N-termini and dAbs specific for another epitope linked to the C-termini (Chames, P. and Baty, D. In: Bispecific Antibodies. Kontermann R E (ed.), Springer Heidelberg Dordrecht London New York, pp. 101-1 14 (201 1)).

Several types of trivalent antibodies have been developed. Tribodies are composed of three distinct scFv regions joined by linker sequences approximately 20 amino acids in length. Tribodies utilize the natural in vivo heterodimerization of the heavy chain (C_{H}1 domain) and light chain (C_{L} domain) to form a scaffold on which multiple scFvs can be added. For example, a scFv specific to one antigen can be linked to a C_{H}1 domain, which is also linked to a scFv specific to another antigen and this chain can interact with another chain containing an scFv specific to either antigen linked to a C_{L} domain (SCFV3-C_{H}1/C_{L}). Another example of a trivalent construction involves the use of a Fab fragment specific to one epitope C-terminally linked to two scFvs specific to another epitope, one on each chain (Fab-scFv2). Yet another example of a trivalent molecule consists of an intact antibody molecule specific to one antigen with a single chain Fab (scFab) linked to the C-terminal end of the molecule (IgG-scFab). The dock-and-lock (DNL) approach has also been used to generate trivalent antibodies (DNL-F(ab)3) (Chang, C.-H. et al. In: Bispecific Antibodies. Kontermann R E (ed.), Springer Heidelberg Dordrecht London New York, pp. 199-216 (201 1)).

Tetravalent antibodies have also been constructed. Examples of tetravalent antibodies include, but are not limited to, scFv2-Fc, F(ab′)2-scFv2, scFv2-H/L, and scFv-dhlx-scFv molecules. Bispecific scFv2-Fc constructs have an Fc domain with two scFvs specific to one molecule linked to the N-termini of the Fc chains and another two scFvs specific to another molecule linked to the C-termini of the Fc chain. Bispecific F(ab′)2-scFv2 constructs include scFv fragments linked to the C-terminal end of an F(ab′) 2 fragment. scFv2-H/L constructs have scFvs specific to one molecule linked to the heavy chains while scFvs specific to another molecule are linked to the light chains. Finally, scFv-dhlx-scFv constructs contain one type of scFv linked to a helical dimerization domain followed by another type of scFv. Two chains of this type can dimerize, generating a tetravalent antibody (Kontermann, R. E. In: Bispecific Antibodies. Kontermann R E (ed.), Springer Heidelberg Dordrecht London New York, pp. 1-28 (201 1)).

### Autoimmune Diseases

In various embodiments, bispecific constructs described herein that target FcRn and an Fcγ or related receptors can be used to treat autoimmune disease, and particularly autoimmune disease mediated by or involving anti-self antibodies that can bind FcRn and Fcγ or related receptors.

As discussed above, treatment of autoimmune disease involving IgG can include high doses of IgG, so called intravenous immunoglobulin (IVIg) therapy, that works at least in part by saturating Fc receptors, thereby interfering with recycling of auto-antibodies. This approach is indiscriminate in respect to the IgGs destabilized, and can result in agammaglobulinemia, which can leave the patient immunosuppressed. An approach that is more specific, e.g., to immunocomplexed IgG can provide the therapeutic benefit of destabilizing immunocomplexed antibodies while preserving monomeric IgG. Thus, therapeutic compositions and methods described herein stem, in part, from the ability to specifically target immune complexed IgG using a bispecific construct that binds both FcRn and a Type I or Type II Fc receptor that are in close (10-100 A preferably 41 A) proximity to each other.

“Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Provided herein is a method of treating an autoimmune disease, which comprises administering an effective amount of a bi- or multi-specific antibody construct specific for FcRn and a Type I or Type II Fc receptor to a patient in need thereof. Non-limiting examples of autoimmune diseases that can be treated include pemphigus (pemphigus vulgaris, pemphigus foliaceus or paraneoplastic pemphigus), Crohn's disease, idiopathic thrombocytopenic purpura (ITP), heparin induced thrombocytopenia (HIT), thrombotic thrombocytopenic purpura (TTP), Myasthenia Gravis (MG), and Chronic Inflammatory Demyelinating Polyneuropathy (CIDP). Additional non-limiting autoimmune diseases include autoimmune thrombocytopenia, immune neutropenia, antihemophilic FVIII inhibitor, antiphospholipid syndrome, Kawasaki Syndrome, ANCA-associated disease, polymyositis, bullous pemphigoid, multiple sclerosis (MS), Guillain-Barre Syndrome, chronic polyneuropathy, ulcerative colitis, diabetes mellitus, autoimmune thyroiditis, Graves' opthalmopathy, rheumatoid arthritis, ulcerative colitis, primary sclerosing cholangitis, systemic lupus erythematosus (SLE), autoimmune encephalomyelitis, Hashimoto's thyroiditis, Goodpasture's syndrome, autoimmune hemolytic anemia, scleroderma with anticollagen antibodies, mixed connective tissue disease, pernicious anemia, idiopathic Addison's disease, autoimmune-associated infertility, glomerulonephritis (e.g., crescentic glomerulonephritis, proliferative glomerulonephritis), insulin resistance, and autoimmune diabetes mellitus (type 1 diabetes mellitus; insulin dependent diabetes mellitus). Autoimmune disease has been recognized also to encompass atherosclerosis and Alzheimer's disease. In another embodiment, the autoimmune diseases include hepatitis, autoimmune hemophilia, autoimmune lymphoproliferative syndrome (ALPS), autoimmune uveoretinitis, glomerulonephritis, agammaglobulinemia, alopecia areata, amyloidosis, ankylosing spondylitis, autoimmune angioedema, autoimmune aplastic anemia, autoimmune dysautonomia, autoimmune hyperlipidemia, autoimmune immunodeficiency, autoimmune inner ear disease (AIED), autoimmune myocarditis, autoimmune pancreatitis, autoimmune retinopathy, autoimmune urticaria, autoimmune urticarial neuropathy, autoimmune axonal neuropathy, Balo disease, Behget's disease, Castleman disease, celiac disease, Chagas disease, chronic recurrent multifocal osteomyelitis (CRMO), Churg-Strauss syndrome, cicatricial pemphigoid, benign mucosal pemphigoid, Cogan's syndrome, cold agglutinin disease, coxsackie myocarditis, CREST disease, essential mixed cryoglobulinemia, dermatitis herpetiformis, dermatomyositis, Devic's disease (neuromyelitis optica), dilated cardiomyopathy, discoid lupus, Dressler's syndrome, endometriosis, eosinophilic angiocentric fibrosis, Eosinophilic fasciitis, Erythema nodosum, Evans syndrome, Fibrosing alveolitis, Giant cell arteritis (temporal arteritis), Hashimoto's encephalitis, Henoch-Schonlein purpura, Herpes gestationis, Idiopathic hypocomplementemic tubulointestitial nephritis, multiple myeloma, multifocal motor neuropathy, NMDA receptor antibody encephalitis, IgG4-related disease, IgG4-related sclerosing disease, inflammatory aortic aneurysm, inflammatory pseudotumour, inclusion body myositis, interstitial cystitis, juvenile arthritis, Kuttner's tumour, Lambert-Eaton syndrome, leukocytoclastic vasculitis, lichen planus, lichen sclerosus, Ligneous conjunctivitis, Linear IgA disease (LAD), Lyme disease, chronic, mediastinal fibrosis, Meniere's disease, Microscopic polyangiitis, Mikulicz's syndrome, Mooren's ulcer, Mucha-Habermann disease, multifocal fibrosclerosis, narcolepsy, optic neuritis, Ormond's disease (retroperitoneal fibrosis), palindromic rheumatism, PANDAS (pediatric autoimmune neuropsychiatric disorders associated with Streptococcus), paraneoplastic cerebellar degeneration, paraproteinemic polyneuropathies, paroxysmal nocturnal hemoglobinuria (PNH), Parry Romberg syndrome, Parsonnage-Turner syndrome, periaortitis, periarteritis, peripheral neuropathy, perivenous encephalomyelitis, POEMS syndrome, polyarteritis nodosa, Type I, II, & III autoimmune polyglandular syndromes, polymyalgia rheumatic, postpericardiotomy syndrome, progesterone dermatitis, primary biliary cirrhosis, psoriasis, psoriatic arthritis, idiopathic pulmonary fibrosis, pyoderma gangrenosum, pure red cell aplasia, Raynaud's phenomenon, reflex sympathetic dystrophy, Reiter's syndrome, relapsing polychondritis, restless legs syndrome, rheumatic fever, Riede's thyroiditis, sarcoidosis, Schmidt syndrome, scleritis, Sjogren's syndrome, sperm and testicular autoimmunity, stiff person syndrome, subacute bacterial endocarditis (SBE), Susac's syndrome, sympathetic ophthalmia, Takayasu's arteritis, Tolosa-Hunt syndrome, transverse myelitis, undifferentiated connective tissue disease (UCTD), vesiculobullous dermatosis, vitiligo, Rasmussen's encephalitis, Waldenstrom's macroglobulinaemia.

Autoimmune diseases can be mediated by IgG, by inappropriately high levels of IgG, auto-reactive IgG, and or immune complex. Non-limiting examples of IgG-mediated autoimmune diseases include Kawasaki disease, Sjogren's disease, Guillain-Barre, inflammatory bowel disease (IBD), Crohn's disease, ulcerative colitis, systemic lupus erythematosus (SLE), lupus arthritis, lupus nephritis, idiopathic thrombocytopenic purpura, rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behget's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, and/or neonatal myasthenia gravis.

In some embodiments, “immune complex” and “immunocomplexed antibody” are used interchangeably. In some embodiments, the immune complex is an immune complex of antigen+antigen-specific antibody. In some embodiments and particularly in some assays as described herein, the immune complex is artificial, i.e., does not occur naturally in the mammal. For example, the immune complex may be a multimeric complex of 4-hydroxy-5-iodo-3-nitrophenyl acetic acid (NIP), chicken ovalbumin (OVA), and an anti-NIP antibody. In this context, the anti-NIP antibody is a chimeric IgG antibody that contains a murine variable region specific for 4-hydroxy-5-iodo-3-nitrophenyl acetic acid and an Fc domain from wild-type human IgG1 (see e.g., Claypool, 2004, Mol. Biol. Cell 15:1746-1759).

### Cancer

In some embodiments, a bispecific or multispecific construct that binds FcRn and a type I or type II Fc receptor targets FcRn and the inhibitory Fc receptor CD32b. Where CD32b generally sends an inhibitory signal upon ligand binding, analogous to well-known checkpoint receptors such as CTLA-4 and PD-1, it can be beneficial to inhibit signaling through the CD32b receptor in order to promote or enhance an immune response, e.g., to treat or enhance an immune response against or against a chronic infection. It is also contemplated herein that a bispecific or multispecific agent as described herein that binds FcRn and CD32b can be administered in combination with one or more checkpoint inhibitors for additional immunostimulatory therapeutic effect. Non-limiting examples of checkpoint inhibitors include inhibitors, often either antibodies against or soluble versions of a checkpoint receptor, selected from inhibitors of PD-1, CTLA-4, LAG-3, TIM-3, and or TIGIT, among others.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments, the cancer is a primary cancer. In some embodiments, the cancer includes metastases in addition to a primary tumor. As used herein, the term “malignant” refers to a cancer in which a group of tumor cells display or have the capacity for uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize. While they can cause damage to surrounding tissue, benign tumors are generally not referred to as “cancer.”

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer is a subject having objectively measurable cancer cells present in the subject's body. Included in this definition are malignant, actively proliferative cancers, as well as potentially dormant tumors or micro-metastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; Merkel cell carcinoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

### Allergy

In some embodiments, a bispecific agent or antibody construct as described herein that binds and inhibits FcRn and a Type I or Type II Fc receptor can target FcRn and FcgR. As described herein, an Fc receptor-mediated disease or disorder can be an allergic disorder. The allergic disorder can be selected from the group consisting of asthma, contact dermatitis, allergic rhinitis, anaphylaxis, and allergic reactions. Methods for treating an allergic disorder comprising administering one or any combination of the effector-deficient bispecific antibodies as described herein are encompassed. The methods can include combination with other therapies.

### Administration

As described herein, serum levels of immunocomplexed antibody can be elevated in autoimmune disease and/or in subjects with autoimmune disease. Accordingly, in one aspect of any of the embodiments, described herein is a method of treating an autoimmune disease in a subject in need thereof, the method comprising administering a bispecific antibody construct as described herein that specifically binds FcRn and a Type I or Type II Fc receptor to a subject suffering from or diagnosed with an autoimmune disease mediated by or involving auto-antibodies, including for example autoreactive IgG. In one embodiment, the methods comprise first measuring or detecting the level of an autoantibody or immune complex, comprising an autoantibody in a subject. The level can be compared to a reference, e.g., a normal baseline or a disease threshold reference level. In one aspect of any of the embodiments, described herein is a method of treating an autoimmune disease in a subject in need thereof, the method comprising: a) determining the level of an auto-antibody and or immunocomplexed antibody in a sample obtained from a subject; and b) administering a bispecific antibody construct to the subject if the level of an auto-antibody and or immunocomplexed antibody is elevated relative to a reference. Alternatively, step b) can comprise not administering a bispecific antibody construct to the subject if the level of an auto-antibody and or immunocomplexed antibody is similar or low relative to a reference.

In some embodiments, the step of determining if the subject has an elevated level of an autoantibody or an immunocomplexed antibody can comprise performing or having performed an assay on a sample obtained from the subject to determine/measure the level of an autoantibody or an immunocomplexed antibody in the subject. In some embodiments of any of the aspects, the step of determining if the subject has an elevated level of an autoantibody or an immunocomplexed antibody can comprise ordering or requesting an assay on a sample obtained from the subject to determine/measure the level of an autoantibody or an immunocomplexed antibody in the subject. In some embodiments of any of the aspects, the step of determining if the subject has an elevated level of an autoantibody or an immunocomplexed antibody can comprise receiving the results of an assay on a sample obtained from the subject to determine/measure the level of an autoantibody or an immunocomplexed antibody in the subject. In some embodiments of any of the aspects, the step of determining if the subject has an elevated level of an autoantibody or an immunocomplexed antibody can comprise receiving a report, results, or other means of identifying the subject as a subject with an elevated level of an autoantibody or an immunocomplexed antibody

In one aspect, a method of treating autoimmune disease in a subject comprises a clinician ordering an assay measuring auto-antibody or immune complex levels in a sample from a patient, and administering a bispecific antibody construct to the patient for whom auto-antibody and or immune complex levels are above a reference level. In another aspect, a method of treating autoimmune disease in a subject comprises a clinician receiving results of an assay on a patient sample reporting autoantibody or immune complex levels above a disease threshold, and the clinician administering a bispecific antibody construct as described herein to the patient.

In one embodiment, a subject who has cancer is treated by administering a bispecific antibody construct that specifically binds FcRn and CD32b. Such treatment can provoke or permit increased anti-tumor activity. Treatments using such a bispecific antibody construct can be administered alone or in combination with other anti-cancer therapies as known to those of ordinary skill in the art. Other anti-cancer therapies include, but are not limited to administration of chemotherapy agents as known in the art, cell-based therapies as known in the art, e.g., chimeric antigen receptor T cell (CAR-T) therapy or dendritic cell vaccines, and/or checkpoint inhibitor therapies, such as anti-PD1, anti-PD-L1, anti-CTLA-4, anti-TIGIT, anti-TIM3, or anti-LAG3 antibodies or soluble receptors or any combination thereof. In some embodiments, treatment with a bispecific antibody construct specific to FcRn and CD32b can expand the range, types and or severity of cancers that are responsive to anti-cancer therapies as described above.

In one embodiment, a subject who has an allergy is treated by administering a bispecific antibody construct that specifically binds to FcRn and CD23. Such treatment can provoke or permit increased anti-allergy activity. Treatments using such a bispecific antibody construct can be administered alone or in combination with other anti-allergy medications or treatments as known to those of ordinary skill in the art. Other anti-allergy medications or treatments include, but are not limited to, administration of anti-inflammatory agents as known in the art, e.g., antihistamines, such as Diphenhydramine (Benadryl), Chlorpheniramine (Chlor-Trimeton), Brompheniramine (Dimetapp, Dimetane), Carbinoxamine (Palgic), Clemastine (Tavist), Cyproheptadine (Periactin), Hydroxyzine (Vistaril) or any combination thereof.

A level which is less than a reference level can be a level which is less by at least about 10%, at least about 20%, at least about 50%, at least about 60%, at least about 80%, at least about 90%, or less relative to the reference level. In some embodiments, a level which is less than a reference level can be a level which is statistically significantly less than the reference level.

A level is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments, a level which is more than a reference level can be a level which is statistically significantly greater than the reference level. In some embodiments of any of the aspects, the reference can be a level of the target molecule in a population of subjects who do not have or are not diagnosed as having, and/or do not exhibit signs or symptoms of an autoimmune disease, cancer, or an allergy. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

The term The term “sample” or “test sample” as used herein denotes a sample taken or isolated from a biological organism, e.g., a blood or plasma sample from a subject. In some embodiments of any of the aspects, the present invention disclosure encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g. isolated at a prior time point and isolated by the same or another person).

In some embodiments, the methods described herein relate to treating a subject having or diagnosed as having an autoimmune disease, cancer, or an allergy with a bispecific antibody construct including binding domains that specifically bind FcRn and a type I or Type II Fc receptor. Subjects having an autoimmune disease, cancer, or an allergy can be identified by a physician using current methods of diagnosing autoimmune disease, cancer, and allergy. Symptoms and/or complications of autoimmune disease, cancer, or allergy which characterize these conditions and aid in diagnosis are well known in the art.

In some embodiments, the methods described herein comprise administering an effective amount of compositions described herein, e.g. a bispecific antibody construct to a subject in order to alleviate a symptom of an autoimmune disease, cancer, or an allergy. As used herein, “alleviating a symptom of an autoimmune disease, cancer, or an allergy” is ameliorating any condition or symptom associated with the autoimmune disease, cancer, or allergy. As compared with an equivalent untreated control, such reduction is by at least 5%, 10%, 20%, 40%, 50%, 60%, 80%, 90%, 95%, 99% or more as measured by any standard technique.

A variety of means for administering the compositions described herein to subjects are known to those of skill in the art. Such methods can include, but are not limited to parenteral, intravenous, intramuscular, subcutaneous, transdermal, airway (aerosol), pulmonary, injection, or intratumoral administration. Administration can be local or systemic.

The term “effective amount” as used herein refers to the amount of bispecific antibody construct that specifically binds FcRn and a Type I or Type II Fc receptor needed to alleviate at least one or more symptom of the disease or disorder, and relates to a sufficient amount of pharmacological composition to provide the desired effect. The term “therapeutically effective amount” therefore refers to an amount of bispecific antibody construct that is sufficient to provide a particular therapeutic effect against an autoimmune disease, cancer, or allergic condition when administered to a typical subject with a given autoimmune disease, cancer, or allergic condition. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of bispecific antibody construct, which achieves a half-maximal inhibition of symptoms) as determined in cell culture, or in an appropriate animal model. The effects of any particular dosage can be monitored by a suitable bioassay, e.g., assay for bispecific antibody construct, among others. The dosage can be determined by a physician and adjusted, as necessary, to suit observed effects of the treatment.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for inflammation, cytokine levels, autoantibodies, tumor growth and/or size, among others. The dosage can be determined by a physician and adjusted, as necessary, to suit observed effects of the treatment.

In some embodiments, the technology described herein relates to a pharmaceutical composition comprising a bispecific antibody construct as described herein, and optionally a pharmaceutically acceptable carrier. In some embodiments, the active ingredients of the pharmaceutical composition comprise a bispecific antibody construct as described herein. In some embodiments, the active ingredients of the pharmaceutical composition consist essentially of a bispecific antibody construct as described herein. In some embodiments, the active ingredients of the pharmaceutical composition consist of a bispecific antibody construct as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) oils, such as peanut oil, cottonseed oil, safflower oil, sesame oil, olive oil, corn oil and soybean oil; (3) glycols, such as propylene glycol; (4) polyols, such as glycerin, sorbitol, mannitol and polyethylene glycol (PEG); (5) buffering agents, such as magnesium hydroxide and aluminum hydroxide; (6) pyrogen-free water; (7) isotonic saline; (8) Ringer's solution; (9) pH buffered solutions; (10) serum component, such as serum albumin, HDL and LDL; (11) C₂-C₁₂ alcohols, such as ethanol; and (12) other non-toxic compatible substances employed in pharmaceutical formulations. Preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a bispecific antibody construct as described herein.

In some embodiments, the pharmaceutical composition comprising a bispecific antibody construct as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a patient, including, but not limited to, DUROS®-type dosage forms and dose-dumping.

Suitable vehicles that can be used to provide parenteral dosage forms of a bispecific antibody construct as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a bispecific antibody construct as disclosed herein can also be incorporated into parenteral dosage forms, including conventional and controlled-release parenteral dosage forms.

Conventional dosage forms generally provide rapid or immediate drug release from the formulation. Depending on the pharmacology and pharmacokinetics of the agent, use of conventional dosage forms can lead to wide fluctuations in the concentrations of the agent in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control an agent's onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing an agent (i.e., going below the minimum therapeutic levels) as well as exceeding the toxicity level for the drug. In some embodiments, the bispecific antibody construct can be administered in a sustained or controlled release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, a bispecific antibody construct described herein is administered as a monotherapy, e.g., another treatment for the autoimmune disease, cancer, or allergy is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramideandtrimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for inflammation according to the methods described herein, the subject can also be administered a second agent and/or treatment known to be beneficial for subjects suffering from inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications, and the like.

In certain embodiments, an effective dose of a composition comprising a bispecific antibody construct as described herein can be administered to a patient once. In certain embodiments, an effective dose of a composition comprising a bispecific antibody construct can be administered to a patient repeatedly. For systemic administration, subjects can be administered a therapeutic amount of a composition comprising a bispecific antibody construct, such as, e.g. 0.001 mg/kg, 0.01 mg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. elevated immunocomplexed antibody, autoantibody, tumor size or growth rate, or, for example allergen-specific IgE, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from monthly, biweekly, weekly, or daily depending on a number of clinical factors including the specific indication and the subject's sensitivity to the bispecific antibody construct. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration monthly, biweekly, weekly, twice weekly, daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a bispecific antibody construct can be administered over a period of time, such as over a 5 minute, 10 minute, 15 minute, 20 minute, or 25 minute period.

The dosage ranges for the administration of a bispecific antibody construct, according to the methods described herein depend upon, for example, the form of the bispecific antibody construct, its potency, and the extent to which symptoms, markers, or indicators of a condition described herein are desired to be reduced, for example the percentage reduction desired for immunocomplexed antibody. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a bispecific antibody construct in, e.g. the treatment of a condition described herein, or to induce a response as described herein (e.g. decreased autoantibody or immunocomplexed antibody, or decreased tumor growth or tumor size, or decreased allergen-specific IgE) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of an autoimmune disease, cancer, or allergy. When using an experimental animal model, efficacy of treatment is evidenced when a statistically significant change in a marker is observed, e.g. immunocomplexed antibody, autoantibody, tumor size, or allergen-specific IgE.

In vitro and animal model assays are provided herein which allow the assessment of a given dose of a bispecific antibody construct. By way of non-limiting example, the effects of a dose of bispecific antibody construct can be assessed by a blood test for immunocomplexed antibody and monomeric antibody, or a tumor biopsy, or a blood test for allergen-specific IgE.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A composition that selectively inhibits interaction between a type I Fc receptor or a type II Fc receptor, FcRn and an immunocomplexed antibody, the composition comprising a first binding domain that specifically binds a human type I Fc receptor or a human type II Fc receptor and a second binding domain that specifically binds a human FcRn.
2. The composition of paragraph 1, wherein the first and/or second binding domains comprise antibody antigen binding domains.
3. The composition of paragraph 1, wherein the first and second binding domains each comprise an antibody antigen binding domain.
4. The composition of paragraph 1, wherein the first and second binding domains are comprised by a human, humanized, or chimeric antibody construct.
5. The composition of paragraph 1, wherein the first and second binding domains are comprised by a bispecific antibody construct.
6. The composition of paragraph 5, wherein the bispecific antibody construct comprises a first binding domain comprising the CDRs of a V_{H}/V_{L} domain pair that specifically binds a human type I Fc receptor or a human type II Fc receptor and a second binding domain comprising the CDRs of a V_{H}/V_{L} domain pair that specifically binds a human FcRn.
7. The composition of paragraph 5, wherein the bispecific antibody construct is selected from the group consisting of a tandem scFv (taFv or scFv₂), diabody, dAb2A/HH2, knob-into-holes bispecific derivative, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH₃, Db-Fc, scFv₂-H/L, DVD-Ig, tandAb, scFv-dhlx-scFv, dAb2-IgG, dAb-IgG, or dAb-Fc-dAb construct.
8. The composition of paragraph 5, wherein the bispecific antibody construct is bivalent, trivalent, or tetravalent.
9. The composition of paragraph 5, wherein the bispecific antibody construct is a diabody or a tribody.
10. The composition of paragraph 6, wherein the V_{H}/V_{L} domain pairs are fused to a non-immunoglobulin scaffold.
11. The composition of paragraph 6, wherein the bispecific antibody construct comprises a DvD-Ig construct.
12. The composition of paragraph 6, wherein the V_{H} of the first V_{H}/V_{L} domain pair is joined to the V_{H} of the second V_{H}/V_{L} domain pair by a linker, and the V_{L} of the first V_{H}/V_{L} domain pair is joined to the V_{L} of the second V_{H}/V_{L} domain pair by a linker.
13. The composition of paragraph 12, wherein the linker is a chemical linker or a polypeptide linker.
14. The composition of paragraph 12, wherein the linker is selected from the group consisting of GGSGGGGSG (SEQ ID NO: 202), GGSGGGGSGGGGS (SEQ ID NO: 204), TVAAP (SEQ ID NO: 203), and TVAAPSVFIFPP (SEQ ID NO: 205).
15. The composition of paragraph 12, wherein the linker positions the first V_{H}/V_{L} domain pair a distance of 10-100 Å away from the second V_{H}/V_{L} domain pair, such that the composition preferentially binds FcRn and FcγR that are complexed with immunocomplexed immunoglobulin.
16. The composition of paragraph 12, wherein the linker positions the first V_{H}/V_{L} domain pair a distance of about 41 Å away from the second V_{H}/V_{L} domain pair.
17. The composition of paragraph 6, wherein the first V_{H}/V_{L} domain pair is on the amino terminus of the bispecific antibody construct or the second V_{H}/V_{L} domain pair on the amino terminus of the bispecific antibody construct.
18. The composition of paragraph 5, wherein the bispecific antibody construct comprises an immunoglobulin constant region.
19. The composition of paragraph 18, wherein the constant region is selected from the group consisting of IgG, IgA, IgD, IgE and IgM immunoglobulin constant regions.
20. The composition of paragraph 18, wherein the constant region is selected from the group consisting of IgG1, IgG2, IgG3 and IgG4 immunoglobulin constant regions.
21. The composition of paragraph 18, wherein the immunoglobulin constant region comprises an ΔE294 mutation, an M428L mutation, an N343S mutation or any combination thereof, wherein the mutation increases circulating half-life of the immunoglobulin.
22. The composition of paragraph 18, wherein the immunoglobulin constant region comprises a C_{H}3 C-terminal lysine deletion (ΔK445) (Lys0) and or an S226P mutation, wherein the mutation stabilizes the immunoglobulin hinge region.
23. The composition of paragraph 5, wherein the bispecific antibody construct comprises an immunoglobulin light chain.
24. The composition of paragraph 5, wherein the immunoglobulin light chain comprises a kappa or lambda light chain immunoglobulin polypeptide.
25. The composition of paragraph 6, wherein the first V_{H}/V_{L} domain pair specifically binds a type I Fc receptor selected from the group consisting of CD32, CD32a, CD32b, CD32c, CD32a^{H}, CD32a^{R}, CD16, CD16a, CD16a^{V158}, CD16a^{F158}, and CD16b.
26. The composition of paragraph 25, wherein the first V_{H}/V_{L} domain pair specifically binds a type II Fc receptor comprising CD23 or DC-SIGN.
27. The composition of paragraph 25, wherein the V_{H}/V_{L} domain pair that specifically binds CD32a binds an epitope or portion of a CD32a epitope selected from the group consisting of VKVTFFQNGKSQKFSRL (SEQ ID NO: 233), VKVTFFQNGKSQKFSHL (SEQ ID NO: 234), and NIGY (SEQ ID NO: 235).
28. The composition of paragraph 25, wherein the V_{H}/V_{L} domain pair that specifically binds CD32b binds an epitope or portion of a CD32b epitope comprising FFQNGKSKKFSRSDPNFSI (SEQ ID NO: 236).
29. The composition of paragraph 25 wherein the V_{H}/V_{L} domain pair that specifically binds CD16a or CD16b binds an epitope or portion of a CD16a or CD16b epitope selected from the group consisting of HKVTYLQNGKDRKYFHH (SEQ ID NO: 237), LVGS (SEQ ID NO: 238), and LFGS (SEQ ID NO: 239).
30. The composition of paragraph 25, wherein the V_{H}/V_{L} domain pair that specifically binds FcRn binds an epitope or portion of an FcRn epitope selected from the group consisting of GPYT (SEQ ID NO: 230), ALNGEE (SEQ ID NO: 231), and DWPEALAI (SEQ ID NO: 232).
31. The composition of paragraph 25, wherein the V_{H}/V_{L} domain pair that specifically contacts CD32a comprises a V_{H} CDR1 (SEQ ID NO: 1-SEQ ID NO: 9), a V_{H} CDR2 (SEQ ID NO: 23-SEQ ID NO: 31), a V_{H} CDR3 (SEQ ID NO: 45-SEQ ID NO: 53), V_{L} CDR1 (SEQ ID NO: 67-SEQ ID NO: 76), a V_{L} CDR2 (SEQ ID NO: 89-SEQ ID NO: 98), and a V_{L} CDR3 (SEQ ID NO: 113-SEQ ID NO: 122).
32. The composition of paragraph 25, wherein the V_{H}/V_{L} domain pair that specifically contacts CD32b comprises a V_{H} CDR1 (SEQ ID NO: 9-SEQ ID NO: 22), a V_{H} CDR2 (SEQ ID NO: 31-SEQ ID NO: 44), a V_{H} CDR3 (SEQ ID NO: 53-SEQ ID NO: 66), V_{L} CDR1 (SEQ ID NO: 76-SEQ ID NO: 88), a V_{L} CDR2 (SEQ ID NO: 98-SEQ ID NO: 112), and a V_{L} CDR3 (SEQ ID NO: 122-SEQ ID NO: 134).
33. The composition of paragraph 25, wherein the V_{H}/V_{L} domain pair that specifically contacts CD16a or CD16b comprises a V_{H} CDR1 (SEQ ID NO: 135-SEQ ID NO: 137), a V_{H} CDR2 (SEQ ID NO: 142-SEQ ID NO: 144), a V_{H} CDR3 (SEQ ID NO: 149-SEQ ID NO: 151), V_{L} CDR1 (SEQ ID NO: 156), a V_{L} CDR2 (SEQ ID NO: 161), and a V_{L} CDR3 (SEQ ID NO: 166).
34. The composition of paragraph 25, wherein the V_{H}/V_{L} domain pair that specifically contacts CD23 comprises a V_{H} CDR1 (SEQ ID NO: 138-SEQ ID NO: 139), a V_{H} CDR2 (SEQ ID NO: 145-SEQ ID NO: 146), a V_{H} CDR3 (SEQ ID NO: 152-SEQ ID NO: 153), V_{L} CDR1 (SEQ ID NO: 157-SEQ ID NO: 158), a V_{L} CDR2 (SEQ ID NO: 162-SEQ ID NO: 163), and a V_{L} CDR3 (SEQ ID NO: 167-SEQ ID NO: 168).
35. The composition of paragraph 25, wherein the V_{H}/V_{L} domain pair that specifically contacts DC-SIGN comprises a V_{H} CDR1 (SEQ ID NO: 140-SEQ ID NO: 141), a V_{H} CDR2 (SEQ ID NO: 147-SEQ ID NO: 148), a V_{H} CDR3 (SEQ ID NO: 154-SEQ ID NO: 155), V_{L} CDR1 (SEQ ID NO: 159-SEQ ID NO: 160), a V_{L} CDR2 (SEQ ID NO: 164-SEQ ID NO: 165), and a V_{L} CDR3 (SEQ ID NO: 169-SEQ ID NO: 170).
36. The composition of paragraph 25, wherein the V_{H}/V_{L} domain pair that specifically contacts FcRn comprises a V_{H} CDR1 (SEQ ID NO: 171-SEQ ID NO: 172), a V_{H} CDR2 (SEQ ID NO: 173-SEQ ID NO: 174), a V_{H} CDR3 (SEQ ID NO: 175-SEQ ID NO: 191), V_{L} CDR1 (SEQ ID NO: 192-SEQ ID NO: 193), a V_{L} CDR2 (SEQ ID NO: 194-SEQ ID NO: 196), and a V_{L} CDR3 (SEQ ID NO: 197-SEQ ID NO: 201).
37. A pharmaceutical composition comprising the composition of any one of paragraphs 1-36 and a pharmaceutically acceptable carrier.
38. A nucleic acid encoding a polypeptide composition of any one of paragraphs 1-37.
39. A vector comprising a nucleic acid encoding a polypeptide composition of paragraph 38.
40. A cell comprising the nucleic encoding a polypeptide composition of paragraph 38 or the vector of paragraph 39.
41. A method for modulating the interaction between a type I Fc receptor or a type II Fc receptor, FcRn and an immunocomplexed antibody, the method comprising contacting a cell with a composition of any one of paragraphs 1-36, a pharmaceutical composition of paragraph 37, a nucleic acid of paragraph 38, a vector of paragraph 39, or a cell of paragraph 40.
42. The method of paragraph 41, wherein the composition does not modulate the binding of FcRn to monomeric antibodies.
43. The method of paragraph 41, wherein modulating the binding of the type I Fc receptor or the type II Fc receptor and FcRn to immunocomplexed IgG occurs at a pH less than 7.
44. A method to inhibit or reduce type I Fc receptor or type II Fc receptor and FcRn interactions with an immunocomplexed antibody, comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof.
45. The method of paragraph 44, wherein the type I Fc receptor is selected from the group consisting of CD32, CD32a, CD32a^{H}, CD32a^{R}, CD16, CD16a, CD16a^{V158}, CD16a^{F158}, and CD16b.
46. The method of paragraph 44, wherein the type II Fc receptor comprises DC-SIGN.
47. The method of paragraph 44, wherein the immunocomplexed antibody comprises an IgG autoantibody.
48. The method of paragraph 44, wherein the level of circulating immunocomplexed IgG autoantibody is reduced.
49. The method of paragraph 44, wherein administration does not result in hypogammaglobulinemia.
50. The method of paragraph 44, wherein innate and adaptive immune responses mediated by FcRn and immunocomplexed antibodies are inhibited or reduced.
51. The method of paragraph 44, wherein the subject has or has been diagnosed with an autoimmune disease, an IgG mediated autoimmune disease and or an inflammatory condition.
52. The method of paragraph 44, wherein the subject has or has been diagnosed with Kawasaki disease, Sjogren's disease, Guillain-Barre, inflammatory bowel disease (IBD), Crohn's disease, ulcerative colitis, systemic lupus erythematosus (SLE), lupus arthritis, lupus nephritis, idiopathic thrombocytopenic purpura, and/or rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behget's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, neonatal myasthenia gravis.
53. A method to reduce the level of circulating immunocomplexed IgG autoantibodies comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein interaction between type I Fc receptor or type II Fc receptor and FcRn with an immunocomplexed antibody is reduced or inhibited.
54. The method of paragraph 53, wherein the type I Fc receptor is selected from the group consisting of CD32, CD32a, CD32a^{H}, CD32a^{R}, CD16, CD16a, CD16a^{V158}, CD16a^{F158}, and CD16b.
55. The method of paragraph 53, wherein the type II Fc receptor comprises DC-SIGN.
56. The method of paragraph 53, wherein administration does not result in hypogammaglobulinemia.
57. A method of treating an autoimmune disease, comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein interaction between type I Fc receptor or type II Fc receptor and FcRn with an immunocomplexed antibody is reduced or inhibited.
58. The method of paragraph 57, wherein the type I Fc receptor is selected from the group consisting of CD32, CD32a, CD32a^{H}, CD32a^{R}, CD16, CD16a, CD16a^{V158}, CD16a^{F158}, and CD16b.
59. The method of paragraph 57, wherein the type II Fc receptor comprises DC-SIGN.
60. The method of paragraph 57, wherein the subject has or has been diagnosed with an autoimmune disease, an IgG mediated autoimmune disease and or an inflammatory condition.
61. The method of paragraph 57, wherein the subject has or has been diagnosed with Kawasaki disease, Sjogren's disease, Guillain-Barré, inflammatory bowel disease (IBD), Crohn's disease, ulcerative colitis, systemic lupus erythematosus (SLE), lupus arthritis, lupus nephritis, idiopathic thrombocytopenic purpura, rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behget's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, and/or neonatal myasthenia gravis.
62. A method to inhibit or reduce CD32b and FcRn interactions with immunocomplexed IgG, comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein the bispecific antibody construct is specific for CD32b and FcRn.
63. The method of paragraph 62, wherein the subject has or has been diagnosed with cancer.
64. The method of paragraph 62, wherein the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer.
65. The method of paragraph 62, wherein administration blocks tolerance and permits anti-tumor immunity.
66. A method of treating cancer comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein the bispecific antibody construct is specific for CD32b and FcRn.
67. The method of paragraph 66, wherein the subject has or has been diagnosed with cancer.
68. The method of paragraph 66, wherein the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer.
69. The method of paragraph 66, wherein administration blocks tolerance and permits anti-tumor immunity.
70. A method to inhibit or reduce CD23 and FcRn interactions with an immunocomplexed IgE, comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein the bispecific antibody construct is specific for CD23 and FcRn
71. The method of paragraph 70, wherein the subject has or has been diagnosed with an IgE-mediated allergy.
72. The method of paragraph 70, wherein the subject has or has been diagnosed with atopic dermatitis, a food allergy, an insect sting allergy, a skin allergy, a pet allergy, a dust allergy, an eye allergy, a drug allergy, allergic rhinitis, a latex allergy, a mold allergy, a sinus infection, or a cockroach allergy.
73. A method of treating an allergy, comprising administering a therapeutically effective amount of a composition of any one of paragraphs 1-36, a pharmaceutical composition of paragraph 37, a nucleic acid of paragraph 38, a vector of paragraph 39, or a cell of paragraph 40 to a subject in need thereof, wherein the bispecific antibody construct is specific for CD23 and FcRn.
74. The method of paragraph 73, wherein the subject has or has been diagnosed with an IgE-mediated allergy.
75. The method of paragraph 73, wherein the subject has or has been diagnosed with atopic dermatitis, a food allergy, an insect sting allergy, a skin allergy, a pet allergy, a dust allergy, an eye allergy, a drug allergy, allergic rhinitis, a latex allergy, a mold allergy, a sinus infection, or a cockroach allergy.

### EXAMPLES

### Example 1

Cellular responses to IgG immune complexes (IC) occur through interactions between the crystallizable fragment (Fc) domain of IgG and a variety of cell associated Fc receptors (FcR) that transport IC and initiate intracellular signals critical for innate and adaptive immune responses. These include the classical so-called type 1 Fc gamma receptors (FcγRs) which in mice and humans are either activating or inhibitory via immunoreceptor tyrosine-based activation or inhibitory motifs (ITAM and ITIM), respectively. In mouse and human, a singular ITIM-bearing FcγR (FcγRIIb) carries out all inhibitory functions. In contrast, multiple activating FcRs in humans (FcγRI or FcγRIIIa/b) and mice (FcγRI, FcγRIII and FcγRIV) function in association with ITAM-bearing common Fγ chain (encoded by human FCER1G and mouse Fcer1g). Unlike mice, humans also express additional activating FcγRs, including FctγRIIa (also known as CD32a, encoded by FCGR2A) and FγRIIc (CD32c), in which the ITAM domain is embedded in the cytoplasmic tail. However, due to a prevalent single nucleotide polymorphism (SNP) in FCGR2C that encodes a stop codon, only 10⁻²⁰% of the human population expresses FγRIIc. CD32a, in contrast, is widely functionally expressed among humans and is expressed constitutively on all myeloid cells, dendritic cells and platelets. Like FcγRIIb/c and FcγRIIIa/b in humans and FcγRIII and FcγRIV in mouse, CD32a (FcγRIIa) is a low affinity IgG receptor that mainly functions to bind IgG IC. The high-affinity FcγRI is not thought to participate in IgG IC responses as it is constitutively saturated with monomeric IgG in vivo. In addition, there are a variety of so-called type 2 RI: receptors that exhibit diverse structures and bind the Fc domain of IgG. These include receptors such as CD23 and DC-SIGN that typically reside on professional antigen presenting cells. Whereas the binding site of type 1 Fcγ receptors overlap with each other, the binding of type 2 Fcγ receptors are unique from type 1 receptors and from each other making them an eclectic group of functional important molecules in IgG biology.

Host responses to IgG IC are also governed by another atypical Fc receptor, the neonatal Fc receptor (FcRn). FcRn consists of a major histocompatibility complex (MHC) class I-related heavy chain (encoded by human FCGRT and mouse Fcgrt) in noncovalent association with P-microglobulin. This heterodimer binds IgG Fc sites distinct from those associated with FcγR binding. Despite its name, FcRn is not restricted to neonates but is functionally expressed throughout life in multiple tissues and cell types and also binds and traffics albumin. A key function of FcRn in these tissues is to mediate the transport and protection of IgG and albumin, resulting in the long half-life of these circulating proteins. In addition, FcRn is expressed in hematopoietic cells including antigen (Ag) presenting cells (APC) such as monocytes, macrophages, dendritic cells (DC), neutrophils and B cells. FcRn in specific APC subsets has recently been recognized to not only protect IgG from catabolism, but also to control IgG IC phagocytosis, to stimulate innate cytokine production such as interleukin (IL)-12, and to engage in more effective MHC class II (MHCII)- and MHC class I (MHCI)-restricted Ag presentation and cross-presentation to CD4⁺ and CD8⁺ T cells, respectively. This is physiologically relevant because the selective absence of FcRn in hematopoietic cells tempers anti-flagellin IgG-driven, DSS-induced colitis, and compromises anti-tumor immunity by preventing colonic DC activation of endogenous tumor-reactive CD8⁺ T cells.

Importantly, many subsets of hematopoietic cells express both FcγRs and FcRn. Further, FcγRs and FcRn can bind IgG in overlapping pH ranges, raising the possibility that these receptors might functionally interact in acidified intracellular environments. As all FcγRs deliver extracellular IgG IC into acidified endosomes in APC where FcRn predominantly resides and functions, it was investigated whether FcRn and FcγRs are co-dependent and interactive receptors in host responses to IgG as an IC. To do so, experiments focused on FcRn's relationship with CD32a to model their interactions with IgG IC.

The function of CD32a was discovered to be dependent upon FcRn and that these two receptors are co-dependent, and their cooperation is mediated by a ternary complex that is bridged by IgG IC at acidic pH as occurs inside a cell that expresses both receptors, notably hematopoietic cells. These results support a sequential model of IgG IC engagement in antigen presenting cells (APC) whereby FcγRs first bind IgG IC at the neutral pH of the cell surface, which initiates cellular signals such as activation of Syk and internalization of IgG IC into intracellular compartments where FcRn resides and which subsequently determines the downstream effects of FcγR engagement through formation of a ternary complex. Abrogation of FcRn function either genetically or pharmacologically abrogates FcγR function.

These studies have also demonstrated that through formation of a ternary complex of FcγR, FcRn and IgG, that FcγR and FcRn come into close proximity. A detailed analysis of these published crystallographic and functional mapping studies have confirmed this and allow the prediction of actual contact sites involved on the surface of FcγR, FcRn and IgG that are involved (see e.g., FIG. 1A-FIG. 1D, FIG. 3A, FIG. 3B, Table 3). In the case of CD32a, a particular area of the receptor was modeled that involves residue 131 (either Histidine or Arginine) that sits in a pocket associated with IgG Fc where it interacts with residues 265 (Aspartic acid), 270 (Aspartic acid) and 267 (Serine). These residues are interestingly shared by all human and mouse IgG subclasses (see e.g., FIG. 2). In addition, the 131 residue of CD32a is shared with all other classical Fcγ receptors. Without wishing to be bound by theory, this led to the hypothesis that generation of a bispecific antibody directed at a particular location on CD32a (as well as CD32b and CD16) together with an antibody directed at the Fc interaction site on FcRn would be capable of selectively blocking the formation of a ternary complex (see e.g., FIG. 4A, FIG. 4B).

Such a reagent is specifically directed at FcRn interactions with IgG immune complexes and not monomeric circulating IgG. Such a therapeutic agent would thus allow for blockade of IgG immune complex effects without causing hypogammaglobulinemia. This would also direct the therapeutic agent more effectively to the target pathways involved making them potentially more effective therapeutics as they will be focused on the relevant cells and mechanisms with greater discrimination. This will be highly differentiating from current anti-FcRn or anti-FcγR therapies.

These data also support bispecific reagents for the treatment of IgG mediated autoimmune diseases (e.g., FcRn-CD32a and FcRn-CD16 bispecific reagents). In addition, a bispecific against FcRn-CD32b would block tolerance and permit anti-tumor immune responses. FcRn-based bispecific antibody constructs can be designed for other type 2 Fc receptors. A bispecific directed at FcRn-CD23 would be useful in allergic disorders and a FcRn-DC/SIGN bispecific would be useful in autoimmunity. To achieve these aims, a FcRn-CD32a specific bispecific antibody is being constructed that will bind FcRn residues and CD32a residues (see e.g., FIG. 4C, Table 4). Similarly, a FcRn-CD16a(b) specific bispecific antibody will be constructed that will bind FcRn residues and CD16a(b) residues (see e.g., FIG. 4C, Table 4).

TABLE 3
Human IgG1Fc residue S267 is critical residue for FcyRs H131/134
binding as observed in various CD16A/B crystal structures

| Name | PDB ID |
|---|---|
| CD16B | 1T83 |
| CD16B | 1E4K |
| CD16B | 1T89 |
| CD16A | 3AY4 |
| CD16A | 3SGJ |
| CD16A | 3SGK |
| CD16A | 5BW7 |
| CD16A | 5D6D |
| CD16A | 3AY4 |

The bispecific antibody constructs described herein are designed to specifically bind to specific residues in FcRn and CD32a, CD32a^{R}, CD32a^{H}, CD32b, CD16a, CD16a⁵, CD16a^{F158}, or CD16b (see e.g., Table 4, FIG. 4C).

TABLE 4
List of FcRn and CD32a, CD32b, CD16a
or CD16b interface residues

| SEQ ID NO: | Target | Sequence |
|---|---|---|
| 230 | FcRn | GPYT |
| 231 | FcRn | ALNGEE |
| 232 | FcRn | DWPEALAI |
| 233 | CD32aR | VKVTFFQN |
| | | GKSQKFSR |
| | | L |
| 234 | CD32aH | VKVTFFQN |
| | | GKSQKFSH |
| | | L |
| 235 | CD32a | NIGY |
| 236 | CD32b | FFQNGKSK |
| | | KFSRSDPN |
| | | FSI |
| 237 | CD16a or | HKVTYLQN |
| | CD16b | GKDRKYFH |
| | | EI |
| 238 | CD16a | LVGS |
| | V158 or | |
| | CD16b | |
| 239 | CD16aF158 | LFGS |

### Example 2

Bispecific Antibody Production

A fully humanized bispecific antibody is being developed with the intent of developing a product for treatment of autoimmune or inflammatory disorders. Components of the bispecific antibody are follows.

FcRn binding can be mediated through SYNT001, a recombinant, humanized, affinity matured IgG4-kappa monoclonal antibody directed against the neonatal Fc receptor (FcRn) at the IgG Fc binding site. SYNT001 contains a C_{H}3 C-terminal lysine deletion (ΔK445) and an S226P mutation to stabilize the hinge region (numbering based on actual SYNT001 amino acid sequence). SYNT001 is intended for treatment of rare autoimmune disorders (see e.g., US publication US 2018/0291101 A1; incorporated herein by reference in its entirety). DNA constructs for SYNT001 are available. Improving the strength of SYNT001's V_{H}/V_{K} association can improve molecule stability and production rates. Production should be greater than 4 gm/L in a medium cycle bioreactor (MCB) for a single V_{H}/V_{K} vector ratio.

The Heavy Chain of SYNT001 (SEQ ID NO: 240) is as follows:

QVQLVQSGAELKKPGASVKLSCKASGYTFTSYGISWVKQATGQGLEWIGE
TYPRSGNTYYNEKFKGRATLTADKSTSTAYMELRSLRSEDSAVYFCARST
TVRPPGIWGTGTTVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLEPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFELYSRLTVDKSRWQEGNVFSCSVMHEALHNHYTQKSLSLSLG

The Light Chain of SYNT001 (SEQ ID NO: 241) is as follows:

DIQMTQSPSSLSASVGDRVTITCKASDHINNWLAWYQQKPGQAPRLLISG
ATSLETGVPSRFSGSGTGKDYTLTISSLQPEDFATYYCQQYWSTPYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

FcγRIIA (CD32a) binding can be mediated through a fully human antibody (produced by the Mederex mouse) that blocks CD32 binding. This antibody, MDE-8, blocks the interaction of IgG with FcγRIIA and has been shown to reduce antibody-induced anemia in a mouse model (see e.g., U.S. Pat. No. 9,382,321; incorporated herein by reference in its entirety). MDE-8 blocks CD32 binding and interaction of aggregated IgG with FcγRIIA. MDE-8 is a Human anti-CD32/IgG1-FcRmut antibody that is transgenic for Human Ig/kappa. MDE-8 is an unusual IgG1, potentially comprising allotype variation, with effector reaction deletion. U.S. Pat. No. 9,382,321 describes multiple effector-deficient anti-CD32 antibodies, including MDE-8 with mutations. Any of the variable domains described in U.S. Pat. No. 9,382,321 can be used for the anti-CD32 specific portion of the bispecific antibody construct.

The Heavy Chain of MDE-8 (SEQ ID NO: 242) is as follows:

QVHLVESGGGVVPGRSLRLSCAASGFTFSSYGMHWVRQAPGKGLEWVAVI
WYDGSNYYYTDSVKGRFTISRDNSKNTLYLQMNSLRAEDTAVYYCARDLG
AAASDYWGQGTLVTVSSASTKGPSVFPLAPSSLSTSGGTAALGCLVKDYF
PEPVTVSWNSGALTSGVHTFPAVLQSSGLYSLSSVVTVPSSSLGTQTYTC
NVNHKPSNTKVDKKVEPKSCDKTHTCPPCPAPELLGGPSVFLEPPKPKDT
LMISRTPEVTCVVVDVSHEDPEVKFNWYVDGVEVHNAKTKPREEQFASTE
RVVSVLTVLHQDWLNGKEYKCKVSNKGLPAPIEKTISKAKGQPREPQVYT
LPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK

The Light Chain of SYNT001 (SEQ ID NO: 243) is as follows:

AIQLTQSPSSLSASVGDRVTITCRASQGINSALAWYQQKPGKAPKLLIYD
ASSLESGVPSRFSGSGSGTDFTLTISSLQPEDFATYYCQQFNSYPHTFGQ
GTKLEIKRTVAAPSVFIFPPSDEQLKGTASVVCLLNNFYPREAKVQWKVD
NALQSGNSQESVTEQDSKDSTYSLSSTLTLSKADYEKHKVYACEVTHQGL
SSPVTKSFNRGEC

Activities include synthesizing a control antibody. The isotype of the control antibody can be IgG1 FcRmut or IgG4 FcRmut, which indicates that the Fc domain contains effector-deficient mutations. A phage library can used to isolate epitope-matched ScFVs. Depending on the bispecific structure, the control antibody can be converted to full mAb format

Without wishing to be bound by theory, it is proposed that a bispecific molecule combining anti-FcRn and anti-CD32 binding domains has superior performance to anti-FcRn antibody in autoimmune diseases with IgG complex component. Importantly, the bispecific molecule does not interact with Fc receptors. The binding domains of the bispecific antibody are derived from two antibodies—SYNT001 and MDE-8. The bispecific antibody construct is first tested with an OVA-NIP whole blood assay (see e.g., Materials and Methods of Example 3).

There are a variety of formats for the bispecific (see e.g., FIG. 5). As two binding domains are in mAb V_{H} and V_{K} format, a Dual Variable Domain (DvD-IG) can be created. Other bispecific formats are also possible. Either the IgG1 FcRmut (e.g., Entyvio) or IgG4-FcRmut, stabilized hinge (e.g., SYNT001) isotype format can be used for the DvD-Ig. IgG1 has extensive clinical track record in bispecific molecules, and a qualified high expression vector is available for IgG1-FcRmut. IgG4-FcRmut is used in SYNT001, and an expression vector with IgG4-FcRmut can be created. The bispecific antibody constructs are first tested with an OVA-NIP whole blood assay (see e.g., Materials and Methods of Example 3). The bispecific antibody constructs can incorporate other bi-specific molecule binding domain and constant region structures (e.g. knob in hole, bivalent Ig, scFV, V_{H}/V_{K} binding domains).

The variable regions of two known human antibodies, SYNT001 (humanized anti-FcRn) and MDE-8 (human anti-CD32), are combined into a Dual-variable domains Ig (DvD-Ig) format with Human IgG1-FcRmut (avoid Fc interactions) or IgG4FcRmut. The two variable regions are combined through a set of linker options—Set 1: GGSGGGGSG (SEQ ID NO: 202) and GGSGGGGSGGGGS (SEQ ID NO: 204) or Set 2-TVAAP (SEQ ID NO: 203) and TVAAPSVFIFPP (SEQ ID NO: 205).

Four bispecific V_{H} and four bispecific V_{K} genes orientations use short linker primers to join the dual V_{H} domains or the dual V_{K} domains.

SYNT001 V_{H} - GGSGGGGSG - MDE-8 V_{H} (short linker)
MDE-8 V_{H} - GGSGGGGSG - SYNT001 V_{H} (short linker)
SYNT001 V_{H} - TVAAP - MDE-8 V_{H} (short linker)
MDE-8 V_{H} - TVAAP - SYNT001 V_{H} (short linker)
SYNT001 V_{K} - GGSGGGGSG - MDE-8 V_{K} (short linker)
MDE-8 V_{K} - GGSGGGGSG - SYNT001 V_{K} (short linker)
SYNT001 V_{K} - TVAAP - MDE-8 V_{K} (short linker)
MDE-8 V_{K} - TVAAP - SYNT001 V_{K} (short linker)

Four bispecific V_{H} and four bispecific V_{K} genes orientations use long linker primers to join the dual V_{H} domains or the dual V_{K} domains.

SYNT001 V_{H} - GGSGGGGSGGGGS - MDE-8 V_{H} (long linker)
MDE-8 V_{H} - GGSGGGGSGGGGS - SYNT001 V_{H} (long linker)
SYNT001 V_{H} - TVAAPSVFIFPP - MDE-8 V_{H} (long linker)
MDE-8 V_{H} - TVAAPSVFIFPP - SYNT001 V_{H} (long linker)
SYNT001 V_{K} - GGSGGGGSGGGGS - MDE-8 V_{K} (long linker)
MDE-8 V_{K} - GGSGGGGSGGGGS - SYNT001 V_{K} (long linker)
SYNT001 V_{K} - TVAAPSVFIFPP - MDE-8 V_{K} (long linker)
MDE-8 V_{K} - TVAAPSVFIFPP - SYNT001 V_{K} (long linker)

The sets of genes are matched for mAb order and linker length. For example, SYNT001 V_{H}-GGSGGGGSG-MDE-8 V_{H} (short linker) matches with SYNT001 V_{K}-GGSGGGGSG-MDE-8 V_{K} (short linker). The V_{K} dual domains and V_{H} dual domains are cloned into the pPBTAK21 (IgG1-FCRmut/Kappa, V_{H}L and V_{K}L) expression vector.

In summary, the following SYNT001/MDE8 DvD-Ig bispecific antibody constructs with either IgG1-FcRmut or IgG4-FcRmut constant regions are being constructed and tested: (a) 5′ SYNT001 Domain with short linker, (b) 5′ SYNT001 Domain with long linker; (c) 5′ MDE-8 Domain with short linker (b) 5′ MDE-8 Domain with long linker. Control MDE8 antibodies with either IgG1-FcRmut or IgG4-FcRmut constant regions are also being constructed and tested.

### Example 3

The Neonatal Fc Receptor (FcRn) Regulates Classical Fcγ Receptor Function and Association with Autoimmunity

IgG autoantibodies and the immune complexes (IC) they form act through classical and atypical Fcγ receptors (FcγR) to drive human autoimmunity, and clinical trials are actively targeting these pathways. Here we show that FcγRIIa (CD32a), a classical FcγR, and the atypical neonatal Fc receptor (FcRn), co-regulate responses to IgG IC by forming a ternary complex bridged by IgG in acidic intracellular compartments in antigen presenting cells. Furthermore, the histidine-131 polymorphism of CD32a (CD32a^{H}), which is associated with human autoimmunity, confers stronger interactions with IgG and FcRn compared to the arginine-131 (CD32aR) variant. Consequently, CD32a^{H} is observed to induce increased innate immune responses, antigen presentation, T cell activation, and sensitivity to FcRn inhibition in response to IgG IC. Thus, immune responses to IgG IC are jointly regulated by CD32a and FcRn and effectively inhibited by FcRn blockade in a CD32a allele-specific manner.

Immunoglobulin gamma (IgG) antibodies contribute significantly to health and disease by modulating the immune system via binding of the Fc region of IgG to numerous ligands and various classical and atypical Fc gamma receptors (FcγR). Although it is well known that classical FcγR, through their activating or inhibitory functions, work in parallel to elicit a balanced immune response, the existence of functional interactions between atypical FcγR and classical FcγR is however unknown. Among the atypical FcγR, the neonatal Fc receptor (FcRn) is noteworthy as a potential partner for classical FcγR in view of its unique mode of binding IgG Fe and primarily intracellular distribution. Specifically, FcRn binds all IgG subclasses at a site on IgG Fc distinct from classical FcγR but only at acidic pH (pH<6.5). In contrast, FcγR binds all subclasses of IgG under both neutral and acidic conditions. Consistent with this mode of binding, FcRn mainly resides within acidic endosomes whereas classical FcγR primarily reside and act on the neutral cell surface. Therefore, most studies of FcRn have focused on its important role in mediating the salvage, recycling and thus protection of IgG from catabolism, which is independent of FcγR. As such, while classical FcγR-deficient mice have been described to possess normal IgG levels, FcRn knockout mice exhibit reduced circulating IgG levels. Further, several clinical trials have also demonstrated that pharmacologic blockade of FcRn in humans decreases circulating levels of monomeric IgG. Additionally, when FcRn is absent in mouse hematopoietic cells in vivo, IgG IC are cleared more rapidly, directly implicating hematopoietically-expressed FcRn as a key regulator of circulating IgG IC (CIC) levels. This important function of FcRn has recently been confirmed to occur in humans wherein pharmacologic blockade of FcRn lowered CIC levels. However, the functional implications of this observation are unknown. In this regard, multiple studies have implicated FcRn in regulating cellular immune responses to IgG IC more commonly attributed to FcγR such as phagocytosis of IgG-opsonized particles, initiation of innate immune responses to IgG IC and regulation of antigen presentation and cross-presentation by antigen presenting cells (APC). This functional convergence of FcRn and FcγR suggests that these receptors may not simply function in parallel and independent pathways but may cooperatively regulate the levels and ability of IgG IC to mediate cellular responses associated with autoimmunity, infections and cancer.

It was thus determined whether FcRn regulates FcγR responses to IgG IC. To do so, experiments focused on FcγRIIa (CD32a), a low-affinity, activating FcγR which is unique to humans and linked to numerous autoimmune diseases such as inflammatory bowel disease (IBD; both Crohn's disease and ulcerative colitis) and rheumatoid arthritis (RA) through undefined mechanisms. Specifically, the gene for CD32a possesses a nonsynonymous, single nucleotide polymorphism (SNP; rs1801274), encoding arginine (R) or histidine (H) at amino acid position 131. Although the CD32a^{R} allele is considered the high responder variant based upon its interactions with mouse (m)IgG1, the CD32a^{H} variant exhibits stronger binding than CD32a^{R} to monomeric human (h)IgG2 and to IC containing hIgG1, hIgG2 and hIgG3. The relationship between CD32a and FcRn in response to IgG IC and the promotion of autoimmune disease was thus investigated.

CD32a and FcRn Form a Ternary Complex with IgG Under Acidic Conditions.

IgG IC are known to be internalized by low affinity FcγR such as CD32a and exhibit prolonged interactions with FcRn in acidic intracellular vesicles that maintain a pH of approximately 5.5. FcRn and CD32aH could simultaneously engage IgG IC under acidic conditions as found in endosomes. This possibility was investigated using an enzyme-linked immunosorbent assay (ELISA) (see e.g., FIG. 10A). All IC described herein consisted of 4-Hydroxy-3-iodo-5-nitrophenylacetyl (NIP) hapten-conjugated ovalbumin (NIP-OVA) complexed with an anti-NIP chimeric IgG. In all cases, the anti-NIP IgG was composed of wild type human (h)IgG1 Fc (or with specified mutation(s)) and murine anti-NIP antigen-binding domain (hereafter “hIgG1^{WT}”) to generate hIgG1^{WT} IC, unless otherwise specified. C-terminus biotinylated CD32a^{H} captured on neutravidin-coated plates were exposed to escalating concentrations of hIgG1^{WT} IC followed by addition of an alkaline phosphatase (ALP)-conjugated hFcRn reporter complex that does not interfere with IgG binding. This demonstrated that FcRn interacted with CD32a^{H} but only in the presence of hIgG1T IC at acidic pH (see e.g., FIG. 6A), consistent with formation of a ternary complex bridged by hIgG1^{WT}. Similar interactions were also observed between FcRn and the CD32a^{R} variant (see e.g., FIG. 6B) and were abolished in both cases if the IgG IC was specifically mutated to lose binding to either FcRn (using anti-NIP hIgG1^{IHH}), or FcγR (using anti-NIP hIgG1N297A) (see e.g., FIG. 6A, FIG. 6B, Table 5). Consistent with this, ICs with murine (m) anti-NIP IgG1, mIgG2a and mIgG2b subclasses also linked the CD32a variants to hFcRn as demonstrated by ELISA (see e.g., FIG. 6C, FIG. 6D). Surface plasmon resonance (SPR) was next used to demonstrate an mIgG-dependent bridge between CD32a and mFcRn (see e.g., FIG. 10B). Co-injection of recombinant mFcRn with mIgG1, mIgG2a and mIgG2b over immobilized CD32a variants at pH 5.5 resulted in additive signals consistent with ternary complex formation (see e.g., FIG. 10C, FIG. 10D). Together, these studies show that mouse and human IgG bridge a complex between both CD32a variants and mFcRn or hFcRn under acidic conditions as occurs in endosomes.

Confocal microscopy was next used to investigate the intracellular proximity of the ternary complex components in APC. Overlap was observed (yellow) between fluorescent hIgG1 IC (gray), CD32a (green) and endogenous mFcRn (red) in CD32a^{H}- or CD32a^{R}-transfected murine RAW264.7 macrophage-like cells (expressing endogenous mFcγR) consistent with co-localization of the three elements of the ternary complex (see e.g., FIG. 10E). In light of these results, crystallographic structures were superimposed from hIgG1 complexed with FcRn or the CD32a variants (see e.g., FIG. 10E). The resulting model predicted a distance of ˜40-50 Å between the CD32a and FcRn binding sites on Fc, a range of distances within which macromolecular interactions can be demonstrated by proximity ligation assay (PLA) techniques. Indeed, PLA in CD32a-expressing mouse RAW264.7 cells demonstrated proximity of both CD32a variants and FcRn within cells when hIgG1 IC were present (see e.g., FIG. 6F, FIG. 10F), consistent with a ternary complex configuration as shown by ELISA and SPR (see e.g., FIG. 6A-D, FIG. 10C).

CD32a requires FcRn for efficient cross-presentation of IgG IC-associated antigens. IgG IC binding to FcRn in endosomes in mouse CD11c⁺ APC induces endosomal recruitment of cellular components associated with antigen processing for presentation and cross-presentation, processes that are important to autoimmunity. As CD32a-IgG-FcRn form a ternary complex at acidic pH, it was next tested whether CD32a-regulation of presentation of IgG IC-born antigens occurs in an FcRn-dependent manner. CD32a^{H} induction of antigen cross-presentation was examined with FcRn-sufficient and FcRn-insufficient IgG IC. Splenic CD11c⁺ APC were isolated from mice Tg for the CD32a^{H} variant of FCGR2 Å and deficient in all endogenous FcγR and the common γ-chain (Fcgr1^{-/-}/Fcgr2b^{-/-}/Fcgr3^{-/-}/Fcer1g^{-/-}, hereafter CD32a^{H-Tg}). This model minimizes any confounding effects of endogenous murine FcγR and allows for direct examination of CD32a. We observed that blocking FcRn with the anti-FcRn monoclonal antibody (mAb) DVN24, but not isotype control antibody, inhibited CD32a^{H-Tg} APC cross-presentation of OVA from hIgG1^{WT} IC to co-cultured CD8+OT-I T cells in a dose-dependent manner as shown by decreased interferon (IFN)γ production by the CD8⁺ T cells (see e.g., FIG. 7A). Similarly, CD32a^{H} was unable toe licit significant cross-presentation of non-FcRn-binding hIgG1IHH IC (see e.g., FIG. 7A). These studies show that CD32a^{H} can induce antigen cross-presentation and that this function depends upon FcRn.

FcRn can Mediate Antigen Cross-Presentation Independently of CD32a.

These data suggest that cellular responses to IgG IC require cooperation between FcγR and FcRn via a ternary complex under acidic conditions. To more closely delineate the role of FcRn in this process, cross-presentation assays were performed with IC formed with anti-NIP hIgG1 containing the Fc mutations MST/HN (hIgG1^{MST/HN}) (see e.g., Table 5). These mutations significantly increase mFcRn binding affinity for IgG at acidic pH (K_{D}=1.2 nM) and also permit IgG binding at neutral pH (K_{D}=7.4 nM), while diminishing CD32a^{H} binding by approximately 50% (see e.g., Table 6). Despite the decreased CD32a^{H} binding, primary APC from CD32a^{H}-Tg mice treated with hIgG1^{MST}/HN IC induced 4-5-fold more IFNγ production by co-cultured OT-I T cells compared to those loaded with hIgG1^{WT} IC (see e.g., FIG. 7B). This response was inhibited by FcRn blockade with DVN24 in a dose-dependent fashion (see e.g., FIG. 7B), indicating that an increase in IgG-FcRn binding can compensate for weakened IgG-CD32a^{H} interactions. The relative contribution of FcRn in cross-presentation were next investigated by assessing FcRn function in the complete absence of FcγR. Although FcRn mostly acts as an intracellular receptor due to its acidic pH requirements, it is present on the APC surface and therefore accessible to extracellular IgG. Thus, mice were derived (see e.g., Table 7) that lacked CD32a, all endogenous FcγR (i.e., Fcgr1^{-/-}/Fcgr2b^{-/-}/Fcgr3^{-/-}) and the ITAM-signaling common Fc γ-chain (Fcer1g^{-/-}) but maintained endogenous mFcRn expression (hereafter “FcγR^{KO}”). Importantly, under physiologic (pH 7.4) extracellular conditions that prevent IgG-FcRn interactions, FcγR deficiency in APC abrogates cross-presentation presumably due to its role in IgG IC internalization and/or early Syk signaling. Nevertheless, when FcγR^{KO} APC were loaded at pH 7.4 with high affinity hIgG1^{MST/HN} IC that can bind surface-expressed FcRn in these conditions, but not hIgG1^{WT} or hIgG^{1HH} IC, induction of IFNγ production was observed from co-cultured OT-I T cells which was inhibited by DVN24 (see e.g., FIG. 7C). Further, when FcγR^{KO} APC were exposed to IC comprised of hIgG1^{WT} IC at pH 5.5, as occurs in certain pathophysiologic contexts, FcRn-dependent induction of IFNγ production by OT-I T cells was also observed (see e.g., FIG. 7D). These studies show that FcRn can permit the antigen presentation machinery independently of FcγR under pathophysiologic conditions, but optimal responses require cooperation between FcγR and FcRn.

CD32a^{H} is more pro-inflammatory and shows greater dependence on FcRn than CD32a^{R}.

A potential mechanism was next investigated to explain the association between the CD32a^{H} variant and autoimmune diseases, and the role played by FcRn given the strong dependence of CD32a^{H} on its function. The role of FcRn was first assessed in determining early signaling responses by CD32a^{H}, which exhibits increased binding to all hIgG1 IC compared to CD32a^{R}. Although CD32a^{H} transfected human embryonic kidney (HEK)293T cells exhibited significantly more phosphorylated-Syk (p-Syk) after binding hIgG1 IC on the cell surface relative to CD32a^{R} expressing HEK293T cells as expected (see e.g., FIG. 11A-FIG. 11E), this was not dependent upon FcRn (see e.g., FIG. 11C-FIG. 11E).

In addition, when the CD32a^{H} and CD32a^{R} variants were examined in antigen presentation in professional APC, which involves events occurring within FcRn-bearing acidic intracellular endosomes, it was observed that RAW264.7 cells expressing CD32a^{H} and treated with an anti-NIP IgG IC induced greater activation of OVA-specific, CD4⁺ T cells in comparison to those expressing CD32a^{R} (see e.g., FIG. 8A, FIG. 8B, FIG. 11F, FIG. 11G). However, and in contrast to p-Syk induction, these antigen presentation events were FcRn-dependent (see e.g., FIG. 8A). Thus, CD32a^{H} exhibits increased FcRn-independent cell surface signaling and FcRn-dependent downstream induction of antigen presentation of hIgG1 IC compared to CD32a^{R}.

As antigen presentation processes involve endosomes and cell-surface FcγR bind IgG IC in acidic environments, CD32a^{H} and CD32a^{R} functions were compared under these conditions. IgG IC binding to CD32a was first assessed on transfected MDCK-II cells in the absence of hFcRn. As extracellular pH decreased from 7.4 to 5.5, CD32a^{H}-transfected MDCK-II cells demonstrated a significant increase in binding to hIgG1 and hIgG2 IC (see e.g., FIG. 8C, FIG. 11H-FIG. 11L). In comparison, CD32a^{R}-expressing MDCK-II cells exhibited little if any augmentation of IgG IC binding under acidic conditions (see e.g., FIG. 8C). This suggested that acidic pH favors CD32a^{H} binding to hIgG1 and hIgG2 IC.

Next, the relative ability of CD32a^{H} or CD32a^{R} to accommodate the ternary complex with IgG and FcRn was investigated using a modification of the ELISA (see e.g., FIG. 10A). By titrating the FcRn reporter complex over fixed, equivalent levels of CD32a-hIgG1 IC complex, the CD32a^{H}-hIgG1 IC exhibited increased binding of hFcRn compared to that associated with a CD32a^{R}-hIgG1 IC (see e.g., FIG. 8D).

These studies indicate that CD32a^{H} exhibits greater interactions with FcRn through increased bridging by hIgG, suggesting that it might be more dependent on FcRn for its function and thus more sensitive to FcRn inhibition during antigen presentation. Indeed, treatment of primary CD11c⁺ CD32a^{H-Tg} and CD32a^{R-Tg} APC over a range of DVN24 concentrations to block FcRn effectively decreased cross-presentation of OVA from hIgG1^{WT} IC (see e.g., FIG. 7A, FIG. 8E). However, IFNγ production was more effectively decreased by FcRn blockade in CD32a^{H-Tg} APC compared to CD32a^{R-Tg} APC (see e.g., FIG. 8E, FIG. 7A, FIG. 11M-FIG. 11O). Together, these studies indicate that the increased ternary complex formation by the CD32a^{H} variant at acidic pH leads to increased FcRn dependence and enhanced sensitivity to FcRn blockade.

CD32a^{H} Exhibits Higher Responses to mIgG1 IC Under Acidic Conditions.

The role of the CD32a-IgG-FcRn ternary complex formation was next investigated in IgG IC-mediated autoinflammatory disease models that involve mIgG. In the context of mIgG1, CD32a^{R} and CD32a^{H} are considered to be “high-responder” and “low-responder” isoforms, respectively, based upon their binding to mIgG1 as a monomer (see e.g., Table 6) and as an IC (see e.g., FIG. 8H, FIG. 8, FIG. 8P-FIG. 8R) at neutral pH. Consistent with this, mIgG1 IC stimulation of CD32a^{R}-transfected HEK293T cells stimulated robust FcRn-independent p-Syk induction, while little p-Syk was observed in CD32a^{H}-transfected HEK293T cells (see e.g., FIG. 8F). However, despite the significantly diminished p-Syk induction by CD32a^{H}, the opposite was observed for cross-presentation of mIgG1 IC. In the latter case, CD32a^{H} expressing HEK293T cells stably expressing H2-Kb induced equivalent or greater levels of IFNγ production by co-cultured CD8+OT-I T cells over a 10-fold range of mIgG1 IC concentrations compared to CD32a^{R}-expressing HEK293T^{H2-Kb} cells (see e.g., FIG. 8G, FIG. 11A, FIG. 11B). These findings were confirmed in primary CD11c⁺ C32a^{H-Tg}D32a^{R-Tg} APC (see e.g., FIG. 811). These data together indicate that IgG IC-FcγR cell surface interactions under physiologic conditions are not be the major factor determining the magnitude of antigen presentation responses to IgG.

Therefore, it was next examined whether ^{CD32aR} and CD32a^{H} interactions with mIgG1 IC also differed within an acidic milieu, where FcRn functions, similar to human IgG IC (see e.g., FIG. 8C). Indeed, mIgG1 IC exhibited greater augmentation of binding to CD32a^{H} expressed on MDCK-II cells relative to CD32a^{R} as extracellular pH decreased from pH 7.4 to 5.5 (see e.g., FIG. 8I, FIG. 11Q-FIG. 11S). Further, CD11c+CD32a^{H-Tg} APC exhibited significantly greater cross-presentation of mIgG1 IC compared to CD32a^{R-Tg} APC in physiologic (pH 7.4) and acidic (pH 5.5) extracellular conditions (see e.g., FIG. 8J). These studies demonstrate that, compared to CD32a^{R}, CD32a^{H} responds more vigorously to mIgG1 IC, which implicates CD32a^{H} as the high-responder variant when assessed by IgG IC binding at acidic pH and APC cross-presentation.

FcRn Blockade Ameliorates IC-Mediated Colitis and RA in a CD32a Allele-Specific Manner.

To demonstrate the relevance of these observations in vivo, a model of IBD, a CD32a^{H}-linked disease, was used with an established DSS-induced colitis model shown to be driven by anti-flagellin IgG and ameliorated by genetic deletion of Fcgrt. Accordingly, bone marrow (BM) was transferred from CD32a^{H-Tg} or CD32a^{R-Tg} CD45.2+mice into irradiated CD45.1+C57BL/6 recipient mice (see e.g., FIG. 12A,IG. 12). CD32a^{Tg} BM chimeric mice were immunized with Salmonella sp. flagellin in incomplete Freund's adjuvant, which primarily induces mIgG1 responses (see e.g., FIG. 12C). Subsequently all groups received DSS in drinking water for 7 days and were treated with low-dose DVN24, starting one day before and continuing through DSS exposure (see e.g., FIG. 12A). Importantly, all treatment groups demonstrated comparable mIgG1 responses to the immunogen (see e.g., FIG. 12C). Further, this low-dose FcRn blockade did not affect the circulating levels of total or flagellin-specific IgG of any subclass compared to isotype control (see e.g., FIG. 9A, FIG. 12C, FIG. 12D). Despite the absence of any effect on circulating anti-flagellin IgG levels, mice with CD32a^{H-Tg} bone marrow treated with DVN24 exhibited significantly less weight loss (see e.g., FIG. 9B), histologic evidence of inflammation (see e.g., FIG. 9C, FIG. 9D), and inflammatory cytokine secretion in colonic tissues or explant cultures (see e.g., FIG. 12E), compared to CD32a^{R-Tg} mice. This IgG driven model of colitis is dependent on hematopoietic cells. Consistent with this, CD11c⁺ APC isolated from mesenteric lymph nodes of the DVN24-treated colitic CD32a^{H-Tg} BM chimeric animals exhibited significantly lower expression of multiple inflammatory mediators compared to isotype-treated controls or DVN24-treated CD32a^{R-Tg} BM chimeric mice (see e.g., FIG. 9E). Thus, DVN24 blockade of the FcRn-IgG interactions decreased inflammation more effectively in the setting of CD32a^{H} compared to CD32a^{R} in a model of IBD consistent with CD32a^{H} being more dependent upon FcRn and sensitive to its blockade.

The generalizability of these findings were next demonstrated in a mouse model of RA, another human disease genetically linked to CD32a^{H} and mediated by pathogenic IgG. The mouse K/BxN model of RA produces an FcRn-dependent inflammatory arthritis induced by transfer of sera containing pathogenic autoreactive IgG from endogenously affected mice. For these studies, bone marrow chimeric mice were prepared and treated with DVN24 as above prior to K/BxN serum transfer (see e.g., FIG. 12F). Although mice expressing both CD32a variants were protected by FcRn antibody blockade, the CD32a^{H-Tg} mice were consistently more protected as compared to the CD32a^{R-Tg} mice, based upon ankle swelling (see e.g., FIG. 9F), clinical inflammation score (see e.g., FIG. 9G, FIG. 12G), joint histopathology (see e.g., FIG. 911, FIG. 9I), and mobility (see e.g., FIG. 9J). A trend towards improvements was further observed in joints erosions of DVN24-treated CD32a^{H-Tg} BM chimeric mice by computerized axial tomography (see e.g., FIG. 12H, FIG. 12I). The importance of FcRn was confirmed in wild-type mice that received bone marrow from CD32a^{Tg} mice genetically sufficient or deficient in FcRn (CD32a^{Tg}/Fcgrt^{-/-}) and treated with K/BxN-derived serum. Although the overall morbidity and mobility (see e.g., FIG. 12J, FIG. 12K) improved with hematopoietic Fcgrt deletion irrespective of the CD32a variant, ankle swelling (see e.g., FIG. 9K) and inflammation scoring (see e.g., FIG. 9L, FIG. 12J) were significantly more improved in the absence of FcRn for the CD32a^{H-Tg} hFCgrt^{-/-}) than CD32a^{R-Tg}/Fcgrt-BM chimeric mice. Collectively, these results demonstrate that CD32a^{H} is more dependent upon FcRn and susceptible to FcRn blockade in vivo in two mIgG1-driven autoimmune disease models.

TABLE 5
Anti-NIP hIgG1 Fc variants and Fc receptor binding characteristics.
Relative binding shown by +, ++, +++; no binding shown by −.

| | | FcRn | | FcγR |
|---|---|---|---|---|
| Variant | Amino acid substitutions | pH 6.0 | 7.4 | 7.4 |
| WT | — | ++ | − | ++ |
| IHH | I253A/H310A/H435A | − | − | ++ |
| N297A | N297A | ++ | − | − |
| MST/HN | M252Y/S254T/T256E/H433K/N434F | +++ | ++ | + |

TABLE 6
CD32a 131 variant binding characteristics with mouse and human IgG
variants. SPR studies were performed with serial dilutions of
IgG variants injected over CD32a
variants at pH 7.4. Estimated steady state K_{D} (μM)
of the monomeric IgG variants' binding to
CD32a variants at pH 7.4

| | CD32a variant | |
|---|---|---|
| IgG variant | H | R |
| hIgG1P^{WT} | 1.3 | 2.0 |
| hIgG2^{WT} | 1.4 | 5.2 |
| hIgG1^{IHH} | 4.8 | 3.7 |
| hIgG1^{MST/HN} | 2.8 | 3.8 |
| mIgG1^{WT} | 8.1 | 0.8 |
| mIgG2a^{WT} | 3.4 | 3.6 |
| mIgG2b^{WT} | 5.0 | 4.5 |

TABLE 7
Transgenic mice. The mouse strains utilized in these studies, as well as
sources and uses are summarized.

| | | | | | | | | Stock | |
|---|---|---|---|---|---|---|---|---|---|
| | | | | | | | | No. | |
| | Strains | Fcrgt | Fcgr1 | Fcgr2b | Fcer1g | FCGR2A | Ptprc | (Source) | Additional information |
| 1 | CD32A ^{R-Tg} | + | − | − | − | + | b | (*) | CD32a^{R} Transgenic, BM Donor |
| 2 | CD32A ^{H-Tg} | + | − | − | − | + | b | (*) | CD32a^{H} Transgenic, BM Donor |
| 3 | CD32^{R-Tg} Fcgrt^{-/-} | − | − | − | − | + | b | (in | Cross #1 × Fcgrt^{-/-} (in house), BM |
| | | | | | | | | house) | Donor |
| 4 | CD32^{H-Tg} Fcgrt^{-/-} | − | − | − | − | + | b | (in | Cross #2 × Fcgrt^{-/-} (in house), BM |
| | | | | | | | | house) | Donor |
| 5 | FcγR^{KO} | + | − | − | − | − | b | (in | mFcγRI/IIB/III/IV deficient, BM |
| | | | | | | | | house) | Donor |
| 6 | CD45.1 | + | + | + | + | NA | a | 4007 (T) | BM Recipients |
| 7 | DO11.10 | + | + | + | + | NA | b | 003303 | For in vitro studies, OVA specific |
| | | | | | | | | (J) | CD4⁺ T cells |
| 8 | OT-I | + | + | + | + | NA | b | 003831 | For in vitro studies, OVA specific |
| | | | | | | | | (J) | CD8⁺ T cells |

(+) gene present,
(−) gene absent,
(NA) not application,
(a) CD45.1,
(b) CD45.2,
(J) Jackson Laboratory,
(T) Taconic Biosciences.

TABLE 8
qPCR primers. 5′ → 3′ sequences of
forward and reverse primers used
in qPCR

| SEQ ID NO: | Primer name: | Sequence: |
|---|---|---|
| 244 | Gzmb FW | TCTTGACGCTGGGACCTAGGCG |
| 245 | Gzmb RV | GGGCTTGACTTCATGTCCCCCG |
| 246 | Cd8a FW | ACTACCAAGCCAGTGCTGCGAA |
| 247 | Cd8a RV | ATCACAGGCGAAGTCCAATCCG |
| 248 | Il10 FW | GAGAGCTGCAGGGCCCTTTGC |
| 249 | Il10 RV | CTCCCTGGTTTCTCTTCCCAAGACC |
| 250 | Tnf FW | CCCTCCTGGCCAACGGCATG |
| 251 | Tnf RV | TCGGGGCAGCCTTGTCCCTT |
| 252 | Il6 FW | TGCAAGAGACTTCCATCCAGTTGCC |
| 253 | Il6 RV | TGTGAAGTAGGGAAGGCCGTGGT |
| 254 | Il12a FW | ACGAGAGTTGCCTGGCTACTAG |
| 255 | Il12a RV | CCTCATAGATGCTACCAAGGCAC |
| 256 | Il12b FW | CCCCTGACTCTCGGGCAGTGAC |
| 257 | Il12b RV | TCTGCTGCCGTGCTTCCAACG |
| 258 | Gapdh FW | GACAGTCAGCCGCATCTTCT |
| 259 | Gapdh RV | GCGCCCAATACGACCAAATC |

It has thus been shown that CD32a and FcRn directly cooperate through formation of a ternary complex on an IgG Fc scaffold under acidic conditions as occurs in intracellular organelles. Consequently, optimal innate immune responses as well as antigen presentation and cross-presentation in response to IgG IC by APC requires both CD32a and FcRn. Thus, pharmacologic blockade of FcRn was observed to disable FcγR-initiated immune responses to IgG IC in association with disease amelioration. In addition, these salutary effects of anti-FcRn therapy in models of IBD and RA were evident without diminution of circulating IgG levels. Together with recent observations that FcRn inhibition decreases CIC and the ability of IgG IC to stimulate innate and adaptive immune responses in humans, these data indicate that inhibiting FcRn interactions with IgG IC may be of central importance in achieving the clinical benefit of FcRn blockade.

These studies also have important implications for current efforts to engineer IgG antibodies with enhanced efficacy. Previous studies have shown that FcRn-enabling ‘LS’ mutations in the Fc region, for example, not only result in an extended half-life of engineered IgG antibodies, but also increase anti-tumor CD8⁺ T cell responses in a mouse model of cancer. Further, studies of passive immunization in nonhuman primates, including those with anti-HIV IgG antibodies engineered to possess ‘LS’-enhanced FcRn binding, surprisingly demonstrated that passive immune protection depends on CD8⁺ T cells. These studies provide an explanation for these observations by suggesting that such FcRn-permitd IgG antibodies can enhance interactions with FcγR through ternary complex formation to enhance cross-presentation and activation of CD8⁺ T cells. Moreover, these FcRn-dependent responses can compensate for diminished or even absent FcγR activity and occur in the absence of FcγR-driven Syk signaling, which is generally considered to be a requirement for cross-presentation. These FcγR-independent functions of FcRn may be particularly important in disease-associated conditions characterized by tissue acidosis, as demonstrated herein. Together, these studies highlight the important contributions that FcRn imparts to IgG IC biology and mechanistically demonstrate that Fc-engineered antibodies with stronger FcRn binding can amplify CD8⁺ T cells responses, which could potentially better treat infections or increase immunopathology.

This characterization of an FcγR-IgG-FcRn ternary complex has further revealed a potential mechanism for the clinically important association between the CD32a^{H} allele and autoimmune disease. While CD32a^{H} could contribute to autoimmune disease through its increased ability to initiate FcRn-independent Syk signaling, consistent with its enhanced binding to human IgG IC subclasses, it was also demonstrated herein that CD32a^{H} more actively promotes FcRn-dependent antigen presentation and T cell activation. The latter occurs through increased propensity of CD32a^{H} to associate with IgG IC under acidic conditions and recruit FcRn to a ternary complex bridged by IgG independent of Syk signaling. Indeed, mIgG1 stimulates significantly greater cross-presentation in the setting of CD32a^{H} despite weaker mIgG1 binding and Syk activation at neutral pH compared to CD32a^{R}. This demonstrates that it is increased recruitment of FcRn to CD32a^{H} under acidic conditions, rather than the magnitude of Syk activation, which drives increased antigen presentation and T cell activation by APC expressing CD32a^{H}. Consequently, CD32a^{H} exhibits augmented FcRn dependence and sensitivity to FcRn inhibition, a crucial translational observation pertinent to ongoing clinical trials and the eventual clinical application of FcRn-targeted therapies. The demonstration of intimate physical and functional interactions between CD32a, IgG IC and FcRn further implicates FcRn in FcγR-mediated diseases and processes more broadly. FcγR are highly polymorphic and linked to a wide variety of infectious and autoimmune diseases. For example, the less stimulatory CD32a^{R} allele carries increased risk of severe infection with encapsulated organisms. These studies indicate that this susceptibility results from relatively weaker FcRn- and IgG IC-dependent ternary complex formation consistent with the role of FcRn in protecting against infection. Conversely, the increased risk of many IgG IC-mediated autoimmune diseases in carriers of the CD32a^{H} variant, implicates enhanced CD32a^{H}-IgG IC-FcRn ternary complex formation in contributing to these types of autoimmunity. FcRn likely forms interactions with other FcγR though an IgG IC bridge. This may be particularly important in polymorphonuclear leukocytes, which express both FcRn and CD16b, an activating FcγR which is monomorphic at position 131 (expressing H) and important for controlling infection and neoplasia. Therefore, many functions of the highly polymorphic FcγR system may funnel into the non-polymorphic FcRn. Thus, the cellular distribution of FcRn and FcγR, and the characteristics of the ternary complexes they form, may significantly impact a variety of immunological functions related to IgG IC and inform efforts to target FcRn in autoimmune disease.

These studies thus reveal the existence of a close coordination between FcγR and FcRn in eliciting antigen presentation and cross-presentation responses to IgG IC and the especially critical role played by FcRn. Further, these studies highlight the importance of allele-specific differences in CD32a coordination with FcRn that delineate important mechanisms by which CD32a^{H} contribute to autoimmune disease.

### Materials and Methods

Animal experiments were approved by IACUC committees. Mice (see e.g., Table 7) were housed in specific pathogen free (SPF) facilities. Wild-type C57BL/6, C57BL/6-Tg(TcraTcrb)1100 Mjb/J (OT-I mice), C.Cg-Tg(DO11.10)10Dlo/J mice were from The Jackson Laboratories, B6.SJL-Ptprc^{a}/BoyAiTac (CD45.1) mice were from Taconic. Fcgrt^{-/-} (mFcRn^{KO}), FCGRT^{TG}/B2M^{TG}/Fcgrt^{-/-} (hFcRn^{TG}), Fcgr1^{-/-}/Fcgr2b^{-/-}/Fcer1g^{-/-} (FcγR^{KO}) and FCGR2A^{R-Tg} (FCGR2A^{R}-Tg/Fcgr1^{-/-}/Fcgr2b^{-/-}/Fcer1g^{-/-}) mice were all previously described. The generation of FCGR2A^{H} mice and derived strains is described below.

Human embryonic kidney (HEK) 293T cells stably express the mouse MHC class I molecule H2-Kb (HEK293TH2-Kb). MDCK-II cells and RAW264.7 cell lines were previously described. The granulocyte macrophage colony-stimulating factor (GM-CSF)-secreting B16-F10 melanoma cell line was previously described. Primary APC and T cells were grown in Rosswell Park Memorial Institute (RPMI)-1640 medium (Corning™) with 10% fetal bovine serum (Life Technologies™), 1% sodium pyruvate (Lonza™), 1% antibiotics (penicillin-streptomycin; Thermo Fisher™), 1% non-essential amino acids (Thermo Fisher™), 8.6 μM β-mercaptoethanol (Sigma-Aldrich™) (hereafter “cRPMI”) at 37° C., 5% CO₂. B16-F10, MDCK-II, HEK293T^{H2-Kb} and all derived cells were grown in complete Dulbecco's modified minimal essential media (DMEM; Corning™) in an environment and with additives as for cRMPI, plus HEPES 1% (Corning™) but without β-mercaptoethanol (hereafter “cDMEM”). Details of cloning and associated primers, as well as methods of transfection and transduction of CD32a variants into these cell types are outlined below.

Proteins and Reagents

4-Hydroxy-3-iodo-5-nitrophenylacetyl (NIP) hapten conjugated-ovalbumin (NIP-OVA) was from Biosearch Technologies™, with 11 NIP molecules per ovalbumin. Standard Flagellin from S. typhimurium was from Invivogen™, and incomplete Freund's Adjuvant from Sigma™. QuickChange II-site directed mutagenesis kit was purchased from Agilent Genomics™. The mAb DVN24 was produced as previously described. Isotype control IgG2a was from BioXCell™ (clone c1.18.4, #BE0085). CD32a staining for flow cytometry was accomplished with the FUN-2 clone (Biolegend™). Mouse mAb clone 11B6 against the cytoplasmic tail of CD32a was from Millipore-Sigma™. Labeling of 11B6 with Alexa Fluor 680 was done as per manufacturer's instructions (Thermo Fisher Scientific™). Rabbit polyclonal antibody against the cytoplasmic tail of rat FcRn was previously described. SYTOX green nuclear acid stain was from Thermo Fisher Scientific™. Saponin was from Sigma-Aldrich™ Proximity ligation assay reagents were Duolink™ In Situ Red Starter Kit Mouse/Rabbit (Sigma-Aldrich™) and Duolink™ In Situ Detection Reagents FarRed (Sigma-Aldrich™). The human IgG1, human IgG2, mouseIgG1, mouseIgG2a, mouse IgG2b (Sigma-Aldrich™) subclasses for cell-binding and confocal microscopy were from human or mouse myeloma with κ-light chains, respectively. In all experiments cell acquisition was performed on MACSQuant™ (Miltenyi Biotec™) or CytoFLEX™ flow cytometer (Beckman Coulter™) and data was analyzed using FlowJo™ software (TreeStar™). Protein concentrations and label incorporation measured use a NanoDrop 2000c™ spectrophotometer (Thermo Fisher™). ELISA plates were analyzed using a VERSAmax™ microplate reader (Molecular Devices™). qPCR reactions were performed using a C1000™ Thermal Cycler with CFX96™ Real-Time System (Bio-Rad™).

Generation of FCGR2A^{H} mice.

CD32a^{H-Tg} mice were geneated by recombineering in EL350 cells as previously described. Homology arms were amplified by PCR, (including 16 kb upstream of Exon 1 and 6 kb downstream of Exon 7 of the FCGR2A^{H} gene), subcloned into a pBeloBAC vector and electroporated into EL350 cells (CTD-2514J12 positive, Invitrogen™) capturing 37 kb of genomic DNA containing the FCGR2 Å locus, as previously described. The presence of rs1801274_His allele of FCGR2 Å was confirmed by DNA sequencing. The resulting captured construct was linearized using NotI restriction enzyme (New England BioLabs™) and microinjected into the pronuclei of fertilized oocytes from C57BL/6 mice. Transgenic FCGR2-AH^{-/-} founders were mated with C57BL/6 mice and maintained on this background. The CD32a^{H-Tg} strain of mice expressing the FCGR2A^{H} transgene as the only FcγR was created by crossing FCGR2A^{H} mice with FcγR^{KO} mice, producing FCGR2A^{H}/Fcgr1^{-/-}/Fcgr2b^{-/-}/Fcer1g^{-/-}, or CD32a^{H-Tg} mice.

Generation of CD32a Variant Expressing Cell Lines

Full-length FCGR2A^{H} cDNA was obtained (Origene™). The CD32a^{R} variant was generated via site-directed mutagenesis using overlapping primer pairs:

Forward-Primer:
(SEQ ID NO: 260)
5′-ATCCCAGAAATTCTCCCGTTTGGATCCCACCTTCT-3′,
Reverse-Primer:
(SEQ ID NO: 261)
5′-AGAAGGTGGGATCCAAACGGGAGAATTTCTGGGAT-3′.

The cDNAs encoding for CD32a^{R} and CD32a^{H} were subcloned into a pcDNA3.1 vector and sequences verified by DNA sequencing. MDCK-II cells, EK293T^{H2-Kb} and RAW264.7 cells were transfected with pcDNA3.1-CD32a^{R}, pcDNA3.1-CD32a^{H} or empty pcDNA3.1 vector using the Lipofectamine™ 2000 reagent (Life Technologies™), TransIT-LT1™ transfection reagent (Mirus Bio™) or by electroporation using the Amaxa Cell line nucleofector Kit V™ (Lonza™), respectively, and were maintained under constant selection pressure by 0.2 mg/ml hygromycin B (Invivogen™). To obtain clones with similar expression of CD32a^{R} or CD32a^{H}, transfected MDCK-II, HEK293T^{H2}-Kb, and RAW264.7 were processed by fluorescence-activated cell sorting (FACS; BD FACSAria II™). SPR and ELISA CD32a-IgG-FcRn binding SPR analysis was performed using a Biacore 3000™ instrument (GE Healthcare™). CM5™ sensor chips (GE Healthcare™) were coupled with neutrAvidin (Pierce™) (˜1000 RU) using amine coupling chemistry, by injecting 5 g/ml in 10 mM sodium acetate, pH 4.5 (GE Healthcare™) at 25° C.). Unreacted moieties were subsequently blocked with 1 M ethanolamine (GE Healthcare™). Sites-specific biotinylated monomeric human CD32a^{H} and CD32a^{R} (Sino Biological Inc™) were captured (˜200 RU) on the neutravidin. PBS containing 0.15 M NaCl and 0.05% Tween 20™ pH 5.5 was used as running buffer and for injections of samples with flow rate 10 μl/min at 25° C. Monomeric anti-NIP IgG variants were injected at 200 nM alone or in the presence of 1 M monomeric His-tagged mouse or human FcRn, produced as previously described. As controls, mFcRn and hFcRn were injected alone.

For steady state affinity measurements, anti-NIP IgG variants (anti-NIP hIgG1, hIgG2, hIgG1-MST/HN, mIgG1, mIgG2a and mIgG2b) were immobilized on CM5 chips using amine coupling chemistry as above (˜1000 RU). Serial dilutions (14 μM-0.1 μM) of monomeric human CD32a^{H} and CD32a^{R} (Sino Biological Inc™) were injected with flow rate 10 μl/min at 25° C. using PBS containing 0.15 M NaCl and 0.05% Tween 20™ pH 5.5 as running and dilution buffer. The binding reactions were allowed to reach (near) equilibrium and K_{D} was derived by nonlinear regression analysis of plots of Req (the equilibrium binding response) versus the IgG concentration using the BIAevaluation 4.1™ Software (GE Healthcare™). For all binding studies, curves were zero adjusted and the reference cell value was subtracted. Regeneration of the surface was done by injection of 10 mM NaOH.

To assess IgG IC bridging of CD32a and FcRn, microtiter wells (Thermo Fisher Scientific™) were coated with 10 μg/ml neutrAvidin (Pierce™) overnight at 4° C., and blocked with 250 μl PBS, 4% skimmed milk (PBS/M) for 1 hour at room temperature. Site-specific biotinylated monomeric human CD32a^{H} and CD32a^{R} (5 μg/ml) (Sino Biological Inc.™) were captured. Serial dilutions of monomeric anti-NIP IgGs or IC containing the anti-NIP IgGs (5 g/ml) in complexed with NIP-OVA (Biosearch Technologies™) at a ratio of 4:1 was added to the wells, incubated for 1 hour and washed. His-tagged hFcRn (2 μg/ml) was added and incubated for 1 hour and then washed. Bound receptor was detected by adding 0.5 μg/ml ALP-conjugated anti-hFcRn nanobody (binds at acidic pH and does not interfere with IgG binding), and after washing, 100 μl p-nitropenylphosphate substrate (Sigma-Aldrich™). The absorbance was measured at 405 nm using a Sunrise™ spectrophotometer (Tecan™).

Production of Recombinant Human and Mouse FcRn

His-tagged soluble mouse and human forms of FcRn was produced using an insect cell based system, as described previously.

Production of Anti-NIP IgG Variants

A vector cassette system (pLNOH2-NIP-IgG-oriP) encoding the constant heavy chain cDNAs from human (IgG1 and IgG2) and mouse IgG subclasses (IgG1, IgG2a, and IgG2b) with specificity for the hapten 5-iodo-4-hydroxy-3-nitro-phenacetyl (NIP) were used to produce full-length recombinant IgG subclasses. In addition, a vector encoding hIgG1^{WT} served as template for sub-cloning of a DNA fragment (synthesized by GenScript™) encoding Fc mutant fragments using the restriction sites AgeI and SfiI (C_{H}2 mutations) or SfiI and BamHI (C_{H}3 mutations) to generate the following variants hIgG-N297 Å (hIgG1^{N297A}), hIgG1-I253A/H310A/H435 Å (hIgG1^{IHH}), hIgG1-M252Y/S254T/T256E/H433K/N434F (hIgG1^{MST/HN}) as previously described (see e.g., Table 5). The anti-NIP IgG antibodies were produced by transient co-transfection of adherent HEK293E cells with the heavy chain-encoding vectors together with a vector encoding the mouse λ light chain with NIP specificity (pLNOH2-NIPλLC-oriP) using Lipofectamine 2000™ (Thermo Fisher™) following the manufacturer's instructions, except for anti-NIP hIgG^{1HH}, which was produced from a stably transfected J558L cell line. The IgG antibodies were purified by affinity (anti-mouse λ L chain CaptureSelect™ column, Thermo Fisher™; or anti-hIgG-C_{H}1 CaptureSelect™ column, Thermo Fisher™, or column coupled with 4-hydroxy-3-nitrophenyl acetyl) and size exclusion chromatography (Superdex™ 200 10/300 column; GE Healthcare™)

Confocal Microscopy and Proximity Ligation Assay

RAW264.7 were seeded onto sterile glass coverslips (12 mm) coated with 0.1 mM poly-L-lysine, at 5×10⁵ cells/ml and incubated overnight at 37° C., 5% CO₂. IC were formed in serum-free DMEM by complexing 0.1 mg/ml hIgG1 (IgG1K from human myeloma plasma; Sigma-Aldrich™) with 0.05 mg/ml Dylight™ 405-conjugated goat F(ab′)2 anti-mouse F(ab′)2 IgG (Jackson ImmunoResearch™) for 60 minutes at 37° C., and then bound to Protein A-conjugated Dynabeads™ (Invitrogen™) by incubation in the dark for 60 minutes at 4° C. Cells were washed with serum-free DMEM, treated with IC-containing DMEM for 30 minutes at 37° C., 5% CO₂ then washed with ice cold PBS and fixed with 3% paraformaldehyde. Permeabilization and blocking was performed with PBS containing 0.1% saponin w/v, 1% bovine serum albumin (BSA) and 5% goat serum for 1 hour at room temperature. Staining overnight at 4° C. for CD32a and mFcRn was performed with antibodies specific for their respective cytoplasmic tails, using mouse IgG1 mAb 11B6 conjugated (5 μg/ml) for CD32a, and unconjugated, mFcRn cytoplasmic tail-specific, rabbit polyclonal antibody (8 μg/ml) for mFcRn, followed by goat anti-rabbit IgG (H+L) cross-absorbed antibody (Thermo Fisher Scientific™) at 1:500 for 30 minutes at room temperature, all in antibody buffer (PBS, 1% BSA, 0.1% saponin). Nuclei were with SYTOX™ green. Cover slips were mounted in Vectashield™ hardset antifade mounting medium (Vector Laboratories™). Images were acquired at 63× under glycerin immersion using an inverted DMi6000™ microscope (Leica™) equipped with a CSU-X1 Yokogawa™ spinning disk, ZYLA SL150 sCMOS™ camera (Andor™), with image analysis and overlay performed with ImageJ™. Proximity ligation assay (PLA) was performed on CD32a variant-expressing RAW264.7 grown on coverslips as for confocal but treated for 15 minutes with fluorescent (DyLight™ 594; Jackson ImmunoResearch™) IC prepared as soluble IC as for confocal microscopy experiments but without Dynabeads™. Cells were then washed, fixed, blocked, permeabilized and stained as for confocal studies, but with unconjugated primary antibodies. Secondary antibodies conjugated to complementary oligonucleotides obtained in the Duolink™ kits (Sigma-Aldrich™) were applied with ligation and polymerization for detection with FarRed™ probes performed with reagents and per instructions supplied by the manufacturer except that the probe step was shortened from 60 to 30 minutes. Imaging and analysis was as above.

IgG IC Cell Binding and APC Functional Studies

For binding studies, IgG-F(ab′)2 IC were formed in serum-free DMEM by complexing hIgG or mIgG from each subclass with Alexa 647-conjugated goat F(ab′)2 anti-human or mouse F(ab′)2 IgG (Jackson ImmunoResearch™) at the indicated concentrations for 60 minutes at 37° C. The human (IgG1 Sigma-Aldrich™) and mouse IgG (IgG1, IgG2a, IgG2b; Sigma-Aldrich™) subclasses were from human or mouse myeloma with κ-light chains. For NIP-OVA Ag presentation and cross-presentation studies, anti-NIP IgG ICs were pre-formed in serum-free RPMI (for primary APC) or DMEM (all others) at 37° C. for 1 hour, mixing every 15 min, and using 100 μg/ml of recombinant anti-NIP hIgG variants and 0.5 μg/ml NIP-OVA, unless otherwise specified.

For binding of IC to surface expressed CD32a^{H} and CD32a^{R}, transfected MDCK-II cells were utilized. All buffers were ice cold and all steps were completed on ice or at 4° C. Human or mouse IgG-F(ab′)2 ICs were formed as described above (for acidic IC binding, DMEM was pH-adjusted with HCl and sterile filtered) and then chilled on ice for 5 min. 10⁵ MDCK-II cells were added to IgG-F(ab′)2 ICs (final IC concentrations as indicated) in a 96 well plate and incubated for 60 minutes at 4° C. Cells were then thoroughly washed (wash buffer was pH-adjusted as appropriate), stained for viability (Fixable Viability Dye™, eBiosciences™) fixed with 2% paraformaldehyde (Electron Microscopy Sciences™) and assessed by flow cytometry. Percent change in IC binding was the quotient of the difference between the relative MFI observed at pH 5.5 and pH 7.4, divided by the starting relative MFI (at pH 7.4). The Syk phosphorylation assay was performed using IgG IC formed as above. CD32a^{R} or CD32a^{H} HEK293T^{H2}-Kb cells were detached and resuspended in warm serum-free DMEM. Cells were mixed with IgG IC (final concentration 5×10⁶ cells/ml with 100 μg/ml IgG and 0.5 μg/ml NIP-OVA) and incubated for 10 minutes at 37° C., then washed with ice-cold PBS, lysed with ice-cold modified lysis buffer (MLB) containing 0.5% (w/v) 3-[(3-Cholamidopropyl)dimethylammonio]-1-propanesulfonate hydrate (CHAPS), 5% (v/v) glycerol, 150 mM NaCl, 2 mM CaCl₂), 25 mM Tris-HCL pH 7.2, and HALT™ Protease and Phosphatase Inhibitor (Thermo Fisher™) Insoluble material was removed by centrifugation, and the supernatant was collected for analysis. After Lysates were precleared with Protein G sepharose slurry (GE Healthcare™) in MLB, protein content was quantified by bicinchoninic acid assay (BCA) (Thermo Fisher™), and immunoprecipitation was performed using Protein G sepharose and 1 μg of mouse anti-Syk IgG antibody 4D10 (Santa Cruz Biotechnology™), overnight at 4° C. on a tube rotator. Unbound material was removed by centrifugation, and the sepharose beads were washed thoroughly with ice-cold MLB. Immunoprecipitated Syk was eluted with 2×SDS-PAGE loading buffer with 5% β-mercaptoethanol and boiling for 10 minutes. After centrifugation, supernatant was removed and resolved on a 4-20% Tris-Glycine SDS-PAGE gel (Thermo Fisher™), followed by wet transfer to a nitrocellulose membrane. After membrane blocking with Odyssey® Blocking Buffer (TBS) (Li-COR™) for 1 hour at RT, immunoblotting for phosphorylated-Syk (p-Syk) was performed using 0.5 μg/ml rabbit anti-phospho Syk (T525/526) polyclonal antibody (Cell Signaling™) with detection by a donkey anti-rabbit-IRDye® 68RD antibody (LI-COR™) Imaging and quantification was performed using a LI-COR Odyssey Fc Imaging System. For quantification of total Syk content of each sample (done without HALT™ Protease and Phosphatase Inhibitor), the p-Syk immunoblot was treated for 20 minutes at RT with Restore™ buffer (Thermo Fisher™) with gentle mixing, then washed, blocked and probed with 1 μg/ml rabbit polyclonal anti-human Syk (Santa Cruz Biotechnology™). Immunoblot detection and quantification was performed as for p-Syk. The p-Syk quantification was normalized for Syk and calculated as a percent of total Syk.

APC Stimulation and Ag Presentation

RAW264.7 macrophages or HEK293T^{H2-Kb} stably expressing CD32a^{R}, CD32a^{H} or pcDNA3.1 control vector were seeded onto a 96 well plate (5×10⁴/well) and incubated in serum-free RMPI with IgG IC prepared as above, at indicated concentrations for 3 hours at 37° C. The cells were then washed and co-cultured with 10⁵ OVA-restricted T cells in complete RMPI per well. CD8⁺ T cells were used for cross-presentation, and CD4⁺ T cells for presentation experiments. Specifically, CD8⁺ T cells from OT-I mice recognizing OVA257-264 peptide in the context of MHCI H-2^{b} were purified using CD8α⁺ T cell Isolation kit (Miltenyi Biotec™) from spleens and peripheral LN from OT-I mice. CD4⁺ T from DO11.10 mice recognizing OVA323-339 peptide in the context of the MHCII H-2^{d} (RAW264.7) were purified using CD4⁺ T cell Isolation kit (Miltenyi Biotec™) from spleens or peripheral LN. The cells were co-cultured in cRPMI and supernatant collected at 24 hours. The levels of IL-2 and/or IFNγ in the co-culture supernatant were quantified by ELISA using OptEIA™ mouse IL-2 or IFNγ ELISA kits according to manufacturer's instructions (BD Biosciences™).

For primary APC studies, ICs were pre-formed in serum-free RPMI medium as described above. CD11c⁺ APC were purified in two steps, first using negative selection (CD19 MicroBeads, Miltenyi™) followed by positive selection (CD11c MicroBeads UltraPure (Miltenyi™), from the spleens of CD32a^{H-Tg} or CD32a^{R-Tg} mice that had been inoculated subcutaneously with 5×10⁶ GM-CSF-secreting B16-F10 melanomas two weeks prior to spleen harvest, as described previously. For FcRn inhibition experiments, APCs were pre-treated for 30 minutes with the indicated concentrations of DVN24 or the IgG2a isotype control prior to IC exposure. APC loading with IgG variant IC occurred by incubation with IC for 3 hours. APCs were washed thrice to remove unbound ICs and then co-cultured for 48 hours with 10′ OT-I cells and supernatant collected for IFNγ quantification as described above.

DSS Colitis Supplemental Studies

BM chimera mice were generated following methods previously described, using 6-week old WT C57BL/6 (CD45.2) or B6.SJL-Ptprc^{a}/BoyAiTac (CD45.1) male mice as BM recipients (see e.g., Table 7). Six weeks after reconstitution, ˜200 μL of venous blood was collected and analyzed by flow cytometry for the BM engraftment. Animals that failed to engraft donated BM were excluded from the study. Flagellin-immunization/DSS-induced colitis model was previously described and performed with the following adjustments. Briefly, after intraperitoneal injection (i.p.) with S. typhimurium in incomplete Freund's adjuvant (IFA; at 1:1 ratio of flagellin:IFA) to immunize four weeks (−28 days) and boost at two weeks (−14 days) prior, DSS (4%) was provided ad libitum in drinking water for the period of 7 days, after which they received normal water. Animal weight was monitored daily throughout. DVN24 or mIgG2a isotype control antibody was administered (0.2 mg/day in 0.2 ml) i.p. on the day prior to DSS and daily. Mice (n=4/group) were sacrificed for collection of blood and tissues on day 9 for cytokine determination and explant culture. Colon biopsies of 1 mm for tissue homogenate were placed in pre-weighed lysing matrix vials (mpbio™) containing PBS with protease inhibitors (Roche™) and snap frozen in liquid nitrogen, and 1 mm colon biopsies for explant culture were placed in 1 ml of cRPMI, 5% CO₂, 37° C. for 24-48 hours). For histopathology, colon was carefully removed on day 11 and the distal 5-7 mm of rectum removed and fixed in 4% formalin. Colitis scoring was performed by a blinded pathologist as previously described. Serum protein content was quantified by Pierce™ bicinchoninic acid (BCA) assay (Thermo Fisher™), and IgG isotype levels were quantified using IgG subtype-specific ELISA kits (Bethyl Biotech™) as previously described. Flagellin-specific IgG was measured as previously described. Total and flagellin-specific IgG subclasses in the serum were quantified using unconjugated goat anti-mouse IgG subclass-specific antibodies (Southern Biotech™; IgG1, IgG2a, IgG2 as primary antibodies). Detection was via a donkey anti-goat-HRP antibody cross-absorbed against mouse IgG (Southern Biotech™), with development by 3,3′,5,5′-Tetramethylbenzidine substrate (TMB) (KPL™). For quantification of tissue IgG in tissue, snap frozen tissue was thawed and homogenized. After removal of insoluble material by centrifugation, cytokine profiles were measured in mouse serum, colonic tissue homogenate and explant culture media by Cytometric Bead Th1/Th2/Th17 and inflammation kit Arrays (BD Biosciences™) as per manufacturer's instruction. Cytokine expression in whole mesenteric LN tissue was analyzed by qPCR (see e.g., Table 8). Mouse IL-2 and IFNγ were quantified by OptEIA™ (BD Biosciences™).

K/BxN RA Model

RA was induced and assessed as described previously, in BM chimeric mice prepared with WT C57BL/6 recipient mice as described above (see e.g., Table 7), with sex matched CD32a^{Tg} donor mice. This experiment was repeated using CD32a^{H-Tg}, or CD32a^{R-Tg} FCRn^{KO}/CD32a^{H-Tg} or FcRn./CD32a^{R-Tg} donor mice. DVN 24 or isotype control IgG2a (0.2 mg in 0.2 ml) was administered i.p. daily beginning on day—1 through day 5 from K/BxN sera injection. After one week (day 7), mobility was assessed by previously described method by counting the number of times a mouse stood per minute to touch the side of a 500 mL glass beaker. Histopathological scoring of rear ankle and knee join inflammation and bone erosion on day was by a blinded pathologist as previously described.

Cross-sectional imaging of forepaws was performed by microcomputed tomography (μCT) using a Scanco mCT-35™ with a 7 mm isotropic voxel size as previously described. The distal ulna, carpal bones, and proximal metacarpals were each given a binary score of 1 or 0 for presence or absence, respectively, of cortical erosion by a blinded radiologist. Each wrist was evaluated independently and scored from 0 to 13 based on the number of involved bones. The sum of scores for right and left forepaws were averaged for each mouse, and treatment/genotype averages then compared for differences.

Structural Models

Superimposition of the FcRn-IgG1 Fc crystal structure (PDB ID: 4N0U) and IgG1 Fc of CD32a^{R} complex (PDB ID: 3RY6) using PyMo™ (DeLano Scientific™) generated a FcRn-IgFc-CD32aR structural model. The Fc from FcRn-IgG1 Fc structure superimposition on the Fc of CD32aR complex exhibited a root mean square deviation (RMSD) of 1.378 Å.

Statistical Analysis

Prism™ for Mac OS X version 7.c™ (GraphPad Software Inc.™) was used for statistical analysis. K_{D} analysis of ELISA binding curves were by non-linear regression using one-site binding kinetics model comparing KD between best fit lines. Non-linear regression analysis of hFcRn ELISA binding curves utilized a 4-parameter fit using least squares with goodness of fit assessed by R, with extra sum-of-squares F test to test detect differences between resulting best-fit curves. Comparisons of two groups was made by Student t test. For three or more groups with two parameters, two-way ANOVA or multiple t test procedures were used. Post-hoc analysis to correct for multiple comparisons and detect differences between groups was by Holm-Sidak or the two-stage linear step-up procedure of Benjamin, Krieger and Yekutieli with false discovery rate <0.05, and Fisher LSD test was used when each comparison stood alone and did not require correction for multiple comparisons. A two-sided probability (P) of alpha error less than 0.05 defined significance.

1. A composition that selectively inhibits interaction between a type I Fc receptor or a type II Fc receptor, FcRn and an immunocomplexed antibody, the composition comprising a first binding domain that specifically binds a human type I Fc receptor or a human type II Fc receptor and a second binding domain that specifically binds a human FcRn.

2.-4. (canceled)

5. The composition of claim 1, wherein the first and second binding domains are comprised by a bispecific antibody construct.

6. The composition of claim 5, wherein the bispecific antibody construct comprises a first binding domain comprising the CDRs of a V_{H}/V_{L} domain pair that specifically binds a human type I Fc receptor or a human type II Fc receptor and a second binding domain comprising the CDRs of a V_{H}/V_{L} domain pair that specifically binds a human FcRn.

7. The composition of claim 5, wherein the bispecific antibody construct is selected from the group consisting of a tandem scFv (taFv or scFv₂), diabody, dAb₂A/HH₂, knob-into-holes bispecific derivative, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb2-IgG, dAb-IgG, or dAb-Fc-dAb construct.

8.-10. (canceled)

11. The composition of claim 6, wherein the bispecific antibody construct comprises a DvD-Ig construct.

12. The composition of claim 6, wherein the V_{H} of the first V_{H}/V_{L} domain pair is joined to the V_{H} of the second V_{H}/V_{L} domain pair by a linker, and the V_{L} of the first V_{H}/V_{L} domain pair is joined to the V_{L} of the second V_{H}/V_{L} domain pair by a linker.

13. (canceled)

14. The composition of claim 12, wherein the linker is selected from the group consisting of GGSGGGGSG (SEQ ID NO: 202), GGSGGGGSGGGGS (SEQ ID NO: 204), TVAAP (SEQ ID NO: 203), and TVAAPSVFIFPP (SEQ ID NO: 205).

15. The composition of claim 12, wherein the linker positions the first V_{H}/V_{L} domain pair a distance of 10-100 Å away from the second V_{H}/V_{L} domain pair, such that the composition preferentially binds FcRn and FcγR that are complexed with immunocomplexed immunoglobulin.

16. The composition of claim 12, wherein the linker positions the first V_{H}/V_{L} domain pair a distance of about 41 Å away from the second V_{H}/V_{L} domain pair.

17. The composition of claim 6, wherein the first V_{H}/V_{L} domain pair is on the amino terminus of the bispecific antibody construct or the second V_{H}/V_{L} domain pair on the amino terminus of the bispecific antibody construct.

18.-24. (canceled)

25. The composition of claim 6, wherein
a. the first V_{H}/V_{L} domain pair specifically binds a type I Fc receptor selected from the group consisting of CD32, CD32a, CD32b, CD32c, CD32a^{H}, CD32a^{R}, CD16, CD16a, CD16a^{V158}, CD16a^{F158}, and CD16b; or
b. the first V_{H}/V_{L} domain pair specifically binds a type II Fc receptor comprising CD23 or DC-SIGN.

26. (canceled)

27. The composition of claim 25, wherein
a. the V_{H}/V_{L} domain pair that specifically binds CD32a binds an epitope or portion of a CD32a epitope selected from the group consisting of VKVTFFQNGKSQKFSRL (SEQ ID NO: 233), VKVTFFQNGKSQKFSHL (SEQ ID NO: 234), and NIGY (SEQ ID NO: 235);
b. the V_{H}/V_{L} domain pair that specifically binds CD32b binds an epitope or portion of a CD32b epitope comprising FFQNGKSKKFSRSDPNFSI (SEQ ID NO: 236); or
c. the V_{H}/V_{L} domain pair that specifically binds CD16a or CD16b binds an epitope or portion of a CD16a or CD16b epitope selected from the group consisting of HKVTYLQNGKDRKYFHH (SEQ ID NO: 237), LVGS (SEQ ID NO: 238), and LFGS (SEQ ID NO: 239).

28.-29. (canceled)

30. The composition of claim 6, wherein the V_{H}/V_{L} domain pair that specifically binds FcRn binds an epitope or portion of an FcRn epitope selected from the group consisting of GPYT (SEQ ID NO: 230), ALNGEE (SEQ ID NO: 231), and DWPEALAI (SEQ ID NO: 232).

31. The composition of claim 25, wherein:
a. the V_{H}/V_{L} domain pair that specifically contacts CD32a comprises a V_{H} CDR1 (SEQ ID NO: 1-SEQ ID NO: 9), a V_{H} CDR2 (SEQ ID NO: 23-SEQ ID NO: 31), a V_{H} CDR3 (SEQ ID NO: 45-SEQ ID NO: 53), V_{L} CDR1 (SEQ ID NO: 67-SEQ ID NO: 76), a V_{L} CDR2 (SEQ ID NO: 89-SEQ ID NO: 98), and a V_{L} CDR3 (SEQ ID NO: 113-SEQ ID NO: 122);
b. the V_{H}/V_{L} domain pair that specifically contacts CD32b comprises a V_{H} CDR1 (SEQ ID NO: 9-SEQ ID NO: 22), a V_{H} CDR2 (SEQ ID NO: 31-SEQ ID NO: 44), a V_{H} CDR3 (SEQ ID NO: 53-SEQ ID NO: 66), V_{L} CDR1 (SEQ ID NO: 76-SEQ ID NO: 88), a V_{L} CDR2 (SEQ ID NO: 98-SEQ ID NO: 112), and a V_{L} CDR3 (SEQ ID NO: 122-SEQ ID NO: 134);
c. the V_{H}/V_{L} domain pair that specifically contacts CD16a or CD16b comprises a V_{H} CDR1 (SEQ ID NO: 135-SEQ ID NO: 137), a V_{H} CDR2 (SEQ ID NO: 142-SEQ ID NO: 144), a V_{H} CDR3 (SEQ ID NO: 149-SEQ ID NO: 151), V_{L} CDR1 (SEQ ID NO: 156), a V_{L} CDR2 (SEQ ID NO: 161), and a V_{L} CDR3 (SEQ ID NO: 166);
d. wherein the V_{H}/V_{L} domain pair that specifically contacts CD23 comprises a V_{H} CDR1 (SEQ ID NO: 138-SEQ ID NO: 139), a V_{H} CDR2 (SEQ ID NO: 145-SEQ ID NO: 146), a V_{H} CDR3 (SEQ ID NO: 152-SEQ ID NO: 153), V_{L} CDR1 (SEQ ID NO: 157-SEQ ID NO: 158), a V_{L} CDR2 (SEQ ID NO: 162-SEQ ID NO: 163), and a V_{L} CDR3 (SEQ ID NO: 167-SEQ ID NO: 168): or
e. the V_{H}/V_{L} domain pair that specifically contacts DC-SIGN comprises a V_{H} CDR1 (SEQ ID NO: 140-SEQ ID NO: 141), a V_{H} CDR2 (SEQ ID NO: 147-SEQ ID NO: 148), a V_{H} CDR3 (SEQ ID NO: 154-SEQ ID NO: 155), V_{L} CDR1 (SEQ ID NO: 159-SEQ ID NO: 160), a V_{L} CDR2 (SEQ ID NO: 164-SEQ ID NO: 165), and a V_{L} CDR3 (SEQ ID NO: 169-SEQ ID NO: 170).

32.-35. (canceled)

36. The composition of claim 6, wherein the V_{H}/V_{L} domain pair that specifically contacts FcRn comprises a V_{H} CDR1 (SEQ ID NO: 171-SEQ ID NO: 172), a V_{H} CDR2 (SEQ ID NO: 173-SEQ ID NO: 174), a V_{H} CDR3 (SEQ ID NO: 175-SEQ ID NO: 191), V_{L} CDR1 (SEQ ID NO: 192-SEQ ID NO: 193), a V_{L} CDR2 (SEQ ID NO: 194-SEQ ID NO: 196), and a V_{L} CDR3 (SEQ ID NO: 197-SEQ ID NO: 201).

37.-40. (canceled)

41. A method for modulating the interaction between a type I Fc receptor or a type II Fc receptor, FcRn and an immunocomplexed antibody, the method comprising contacting a cell with a composition of claim 1.

42. The method of claim 41, wherein the composition does not modulate the binding of FcRn to monomeric antibodies.

43. The method of claim 41, wherein modulating the binding of the type I Fc receptor or the type II Fc receptor and FcRn to immunocomplexed IgG occurs at a pH less than 7.

44.-56. (canceled)

57. A method of treating an autoimmune disease, comprising administering a therapeutically effective amount of a composition comprising a first binding domain that specifically binds a human type I Fc receptor or a human type II Fc receptor and a second binding domain that specifically binds a human FcRn to a subject in need thereof, wherein interaction between type I Fc receptor or type II Fc receptor and FcRn with an immunocomplexed antibody is reduced or inhibited.

58.-65. (canceled)

66. A method of treating cancer comprising administering a therapeutically effective amount of a composition comprising a first binding domain that specifically binds a human type I Fc receptor or a human type II Fc receptor and a second binding domain that specifically binds a human FcRn, wherein the composition is specific for CD32b and FcRn.

67.-75. (canceled)
